(19) United States
(12) Patent Application Publication
(10) Pub. No.: US 2009/0131430 A1
(43) Pub. Date: May 21, 2009

(54) PYRIDO'2,3-DIPYRIMIDINES AS ANTI-INFLAMMATORY AGENTS

(76) Inventors: Venkata P. Palle, Gurgaon (IN); Rakesh Kumar Singh, Delhi (IN); Sanjay Malhotra, New Delhi (IN); Yogesh Bhaskarrao Waman, Nashik (IN); Ashwani Verma, New Delhi (IN); Abhijit Ray, New Delhi (IN); Geeta Sharma, New Delhi (IN)

Correspondence Address: RANBAXY INC., 600 COLLEGE ROAD EAST, SUITE 2100, PRINCETON, NJ, 08540, US

(21) Appl. No.: 11/720,047

(22) PCT Filed: Nov. 23, 2005
(86) PCT No.: PCT/IB2005/003523

§ 371 Date: May 27, 2008

Related U.S. Application Data
(60) Provisional application No. 60/630,517, filed on Nov. 23, 2004.

(51) Int. Cl. A61K 31/5377; C07D 471/04; A61P 29/00; A61K 31/519

(52) U.S. Cl. 514/234.2; 544/279; 514/264.11; 544/117

(57) ABSTRACT

The present invention relates to novel azabicyclo derivatives as anti-inflammatory agents. The compounds provided herein can be useful for inhibition and prevention of inflammation and associated pathologies including inflammatory and autoimmune diseases such as sepsis, rheumatoid arthritis, inflammatory bowel disease, type-1 diabetes, asthma, chronic obstructive pulmonary disorder, organ transplant rejection and psoriasis. Also provided herein are pharmacological compositions containing compounds provided herein and associated methods of treating sepsis, rheumatoid arthritis, inflammatory bowel disease, type-1 diabetes, asthma, chronic obstructive pulmonary disorder, organ transplant rejection and psoriasis, and other inflammatory and/or autoimmune disorders, using the compounds.

### FIELD OF THE INVENTION

The present invention relates to novel azabicyclo derivatives as anti-inflammatory agents.

The compounds provided herein can be useful for inhibition and prevention of inflammation and associated pathologies including inflammatory and autoimmune diseases such as sepsis, rheumatoid arthritis, inflammatory bowel disease, type-1 diabetes, asthma, chronic obstructive pulmonary disorder, organ transplant rejection and psoriasis.

Also provided herein are pharmacological compositions containing compounds provided herein and associated methods of treating sepsis, rheumatoid arthritis, inflammatory bowel disease, type-1 diabetes, asthma, chronic obstructive pulmonary disorder, organ transplant rejection and psoriasis, and other inflammatory and/or autoimmune disorders, using the compounds.

### BACKGROUND OF THE INVENTION

During the last decade, studies have focused on the roles played by cytokines, a unique class of intercellular regulatory proteins, in the pathogenesis of many diseases. Cytokines play a role in initiating, maintaining, and regulating immunological and inflammatory processes. Advances in our understanding of their role in immune and inflammatory disorders have led to the development of cytokine-based therapies, that is, therapies that aim to modulate the activity of specific cytokines. Today, drugs that block inflammatory cytokines, such as tumor necrosis factor-alpha (TNF-α), are being introduced to the market.

Elevated levels of proinflammatory cytokines viz TNF-α and IL-1α are associated with the pathogenesis of many immune mediated inflammatory disorders like sepsis, rheumatoid arthritis, inflammatory bowel disease, type-1 diabetes, asthma, chronic obstructive pulmonary disorder, organ transplant rejection and psoriasis. Inflammation is regulated by pro- and anti-inflammatory mediators, which include cytokines, eicosanoids, nitric oxide, and reactive oxygen species. The role of these inflammatory mediators in the pathogenesis of both chronic and acute inflammatory diseases is documented. Until a few years ago, inflammatory disorders were treated primarily with relatively non-selective anti-inflammatory agents, such as corticosteroids and various non-steroidal anti-inflammatory drugs. In recent years, novel therapies have been developed that specifically interfere with the action of selected pro-inflammatory mediators, such as TNFα and PGE2. These specific anti-inflammatory therapies have been used for the treatment of rheumatoid arthritis, inflammatory bowel disease, and several other inflammatory diseases.

The protein-based therapies that inhibit the activities of tumour-necrosis factor-α (TNF-α), including etanercept (Enbrel; Amgen/Wyeth), infliximab (Remicade; Centocor), and adalimumab (Humira; Abbott), have been used for the treatment of autoimmune diseases such as rheumatoid arthritis. However, current injectable therapies have associated limitations and risks, including the potential for increased malignancies and infections and increased congestive heart failure. Studies in rodent models have provided evidence that targeting specific pathways involved in TNF-α activities are effective approaches to interrupting the pro-inflammatory process. Oral small molecules that regulate these pathways could be the next significant advancement in the treatment of chronic inflammatory diseases when used either as a monotherapy or in combination with the current injectables.

Studies have now established that the pathogenesis of inflammatory diseases utilizes cytokine-mediated communication between endothelial cells, infiltrating leukocytes, resident macrophages, mast cells, epithelial cells and osteoclasts. The p38 mitogen activated protein kinase (p38MAPK) regulates cytokine levels and therefore plays a central role in both the cellular infiltration and activation responses associated with inflammatory diseases.

The p38 MAPK is a member of a large family of MAPK's whose signaling pathways also include the extracellular regulated kinases (ERK) & the c-jun N terminal kinases (JNK). MAP kinases are Serine Threonine Kinases that transduce environmental stimuli to the nucleus and they themselves are activated by upstream MAPK kinases by phosphorylation on both Tyrosine and Threonine residues. The MAPK pathways are involved in alterations in cell physiology resulting from a variety of stimuli and control cell death, cell cycle machinery, gene transcription and protein translation. p38α MAPK was first identified as a tyrosine phosphorylated protein in LPS (Lipopolysaccharide) stimulated macrophages. The human p38α MAPK was identified as a target of pyridinyl imidazole compounds (cytokine suppressive anti-inflammatory drugs) that were known to block TNF-α and IL-1 release from LPS stimulated monocytes. After the cloning of first p38MAPK (p38α), additional members of the p38MAPK family were cloned by homology, including the p38α, p38β and p38γ.

The p38 pathway controls the activity of multiple transcription factors and the expression of many genes. There is ample evidence implicating a role for p38 in inflammatory processes mediated by IL-1 and TNF-α. Further, p38 inhibitors have been shown to effectively block both TNFα and IL-1 biosynthesis by LPS stimulated human monocytes. In addition, p38MAPk also plays a role in the production of IL-4, IL-6, IL-8 and IL-12. p38MAPk is also critical for cell response to certain cytokines. Treatment of human neutrophils with GM-CSF, TNF-α or TGF-α results in p38 activation. GM-CSF and TNF-α are potent enhancers of neutrophil respiratory activity suggesting a role for p38MAPk in respiratory burst.

p38 has also been implicated in the induction of cyclooxygenase-2 (COX-2) in LPS-induced monocytes. COX-2 enzyme is the key enzyme in the production of prostaglandins from arachidonic acid. Inhibitors of p38MAP kinase are also expected to inhibit COX-2 expression. Accordingly, inhibitors of cytokine synthesis would be expected to be effective in disorders currently treated with NSAID's. These disorders include acute and chronic pain as well as symptoms of inflammation and cardiovascular disease.

Compounds which modulate release of one or more of the aforementioned inflammatory cytokines can be useful in treating diseases associated with the release of these cytokines.

PCT Application WO 01/44258 discloses bone-targeting groups described as useful for treating a variety of disorders and conditions. PCT Application WO 02/18380, and U.S. Pat. No. 6,518,276 and U.S. Pat. No. 6,506,749 disclose 7-oxopyridopyrimidines said to be inhibitors of cell proliferation. PCT Application WO 03/057165 describes the compositions and methods for prevention and treatment of amyloid-β-peptide related disorders. U.S. Pat. No. 6,316,464 discloses compounds as possible p-38 kinase inhibitors. U.S. Pat. No. 6,451,804 discloses heteroalkylamino-substituted bicyclic nitrogen heterocycles. U.S. Pat. No. 6,696,566 discloses 6-substituted pyrido-pyrimidines described as useful for the treatment of p-38 mediated disorders. U.S. Pat. No. 6,479,507 discloses p-38 kinase inhibitors. U.S. Application 2003/0153586 discloses 7-oxo-pyridopyridopyrimidines said to be useful for the treatment of p-38 mediated disorders. U.S. Pat. No. 6,630,485 discloses p-38 kinase inhibitors, pharmaceutical compositions containing them, method for their use, and methods for preparing these compounds.

### SUMMARY OF THE INVENTION

Azabicyclo derivatives, which can be used for the for inhibition and prevention of inflammation and associated pathologies such as sepsis, rheumatoid arthritis, inflammatory bowel disease, type-1 diabetes, asthma, chronic obstructive pulmonary disorder, organ transplant rejection and psoriasis are provided herein. Pharmaceutically acceptable salts, pharmaceutically acceptable solvates, enantiomers, diastereomers or N-oxides of these compounds having the same type of activity are also provided. Pharmaceutical compositions containing the compounds, and which may also contain pharmaceutically acceptable carriers or diluents, which may be used for the treatment of inflammatory and autoimmune diseases such as such as sepsis, rheumatoid arthritis, inflammatory bowel disease, type-1 diabetes, asthma, chronic obstructive pulmonary disorder, organ transplant rejection and psoriasis are also provided.

Other aspects will be set forth in accompanying description which follows and in part will be apparent from the description or may be learnt by the practice of the invention.

In accordance with one aspect, there is provided a compound having the structure of Formula I

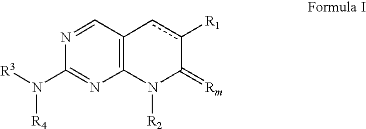

and its pharmaceutically acceptable salts, pharmaceutically acceptable solvates, esters, enantiomers diastereomers, N-oxides, polymorphs, metabolite.

R₁ can be alkyl, alkenyl, alkynyl, cycloalkyl, aryl, aralkyl, heteroaryl, heterocyclyl, heteroarylalkyl, or heterocyclylalkyl.

When R₁ is oxygen or sulphur, R₂ can be alkyl, alkenyl, alkynyl, cycloalkyl, aryl, aralkyl, heteroaryl, heterocyclyl, heterocyclylalkyl or heteroarylalkyl.

When Rₘ is —NH, —N-acyl, —N(CN), —N(NO₂), —C(R₃)₂ or —CH(NO₂), R₂ can be hydroxy, alkoxy, aryloxy, —CHO, —CN, alkyl, alkenyl, alkynyl, cycloalkyl, carboxy, halogen, aryl, aralkyl, acyl, heteroaryl, heterocyclyl, —SO₂R₅, —COOR₆, —C(═O)NRₓR_{y}, ⁻NRₓR_{y} or —OC(═O)NRₓR_{y}, —NHC(═O)Rₓ.

The symbol [symbol] represents a single bond or a double bond.

R₃ can be hydrogen, alkyl, alkenyl, alkynyl, cycloalkyl, aryl, heterocyclyl, heteroaryl, aralkyl, heteroarylalkyl or heterocyclylalkyl.

R₄ can be

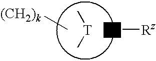

(wherein

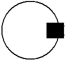

represents a cyclic ring having 4 or 5 carbon atoms, k is an integer selected from 0-2, M is O or N, and T is —(CH₂)ₙ—, —CH(O)CH₂—, —CH₂CH(O)CH₂—, —CH(O)—, —CH₂—O—CH₂—, —CH₂—NH—CH₂—).

R_{z} can be no atom (when M is oxygen), hydrogen or Rᵤ (wherein Rᵤ can be hydroxy, alkoxy, aryloxy, —CHO, —CN, alkyl, alkenyl, alkynyl, cycloalkyl, carboxy, halogen, aryl, aralkyl, acyl, heteroaryl, heterocyclyl, —SO₂R₅, —COOR₆, —C(═O)NRₓR_{y}, —NRₓR_{y} or —OC(═O)NRₓR_{y} or —NHC(═O)Rₓ).

n can be an integer selected from 0-3 (wherein when n is zero then T represents a direct bond).

R₅ can be alkyl, alkenyl, alkynyl, cycloalkyl, —NRₚR_{q} (wherein Rₚ and R_{q} can be hydrogen, alkyl, alkenyl, alkynyl, cycloalkyl, aryl, aralkyl, heterocyclyl, heteroaryl, heterocyclylalkyl or heteroarylalkyl, or Rₚ and R_{q} may also together join to form a heterocyclyl ring), aryl, aralkyl, heteroaryl, heterocyclyl, heterocyclylalkyl or heteroarylalkyl.

R₆ can be alkyl, alkenyl, alkynyl, cycloalkyl, aryl, aralkyl, heteroarylalkyl or heterocyclylalkyl.

Rₓ and R_{y} can be hydrogen, alkyl, alkenyl, alkynyl, cycloalkyl, aryl, aralkyl, —SO₂R₅ (wherein R₅ is the same as defined above), heteroaryl, heterocyclyl, heteroarylalkyl or heterocyclylalkyl.

Q can be alkyl, alkenyl, alkynyl, cycloalkyl, aryl, heteroaryl, heterocyclyl, aralkyl, heteroarylalkyl or heterocyclylalkyl.

In accordance with a second aspect, there are provided methods for the treatment of mammal suffering from inflammation and associated pathologies.

In accordance with a third aspect, there are provided methods for the treatment of mammal suffering from inflammatory diseases and associated pathologies including sepsis, rheumatoid arthritis, inflammatory bowel disease, type-1 diabetes, asthma, chronic obstructive pulmonary disorder, organ transplant rejection and psoriasis.

In accordance with a fourth aspect, there are provided pharmaceutical compositions containing the compounds, and which may also contain pharmaceutically acceptable carriers or diluents, which may be used for the treatment of inflammatory and autoimmune diseases such as such as sepsis, rheumatoid arthritis, inflammatory bowel disease, type-1 diabetes, asthma, chronic obstructive pulmonary disorder, organ transplant rejection and psoriasis.

In accordance with a fifth aspect, there are provided processes for the preparation of compounds disclosed herein.

In accordance with a sixth aspect, the compounds disclosed herein are screened as p38 kinase inhibitors.

The following definitions apply to terms as used herein:

The term “alkyl,” unless otherwise specified, refers to a monoradical branched or unbranched saturated hydrocarbon chain having from 1 to 20 carbon atoms. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, acyl, acylamino, acyloxy, alkoxycarbonylamino, azido, cyano, halogen, hydroxy, oxo, thiocarbonyl, carboxy, carboxyalkyl, aryl, heterocyclyl, heteroaryl, arylthio, thiol, alkylthio, aryloxy, nitro, aminosulfonyl, aminocarbonylamino, —NHC(═O)R_{f}, —NR_{f}R_{q}, —C(═O)NR_{f}R_{q}, —NHC(═O)NR_{f}R_{q}, —C(═O)heteroaryl, C(═O)heterocyclyl, —O—C(═O)NR_{f}R_{q} {wherein R_{f} and R_{q} are independently selected from alkyl, alkenyl, cycloalkyl, cycloalkenyl, aryl, aralkyl, heterocyclyl, heteroaryl, heterocyclylalkyl, heteroarylalkyl}, nitro, or —SO₂R₆ (wherein R₆ is alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, carboxy, —NR_{f}R_{q}, —C(═O)NR_{f}R_{q}, —OC(═O)NR_{f}R_{q}, —NHC(═O)NR_{f}R_{q} (wherein R_{f} and R_{q} are the same as defined earlier), hydroxy, alkoxy, halogen, CF₃, cyano, and —SO₂R₆, (wherein R₆ are the same as defined earlier); or an alkyl group also may be interrupted by 1-5 atoms of groups independently selected from oxygen, sulfur or —NRₐ— {wherein Rₐ is selected from hydrogen, alkyl, cycloalkyl, alkenyl, cycloalkenyl, alkynyl, aryl, acyl, aralkyl, —C(═O)OR_{f} (wherein R_{f} is the same as defined earlier), SO₂R₆ (where R₆ is as defined earlier), or —C(═O)NR_{f}R_{q} (wherein R_{f} and R_{q} are as defined earlier)}. Unless otherwise constrained by the definition, all substituents may be substituted further by 1-3 substituents selected from alkyl, carboxy, —NR_{f}R_{q}, —C(═O)NR_{f}R_{q}, —O—C(═O)NR_{f}R_{q} (wherein R_{f} and R_{q} are the same as defined earlier) hydroxy, alkoxy, halogen, CF₃, cyano, and —SO₂R₆ (where R₆ is same as defined earlier); or an alkyl group as defined above that has both substituents as defined above and is also interrupted by 1-5 atoms or groups as defined above.

The term “alkenyl,” unless otherwise specified, refers to a monoradical of a branched or unbranched unsaturated hydrocarbon group having from 2 to 20 carbon atoms with cis, trans, or geminal geometry. In the event that alkenyl is attached to a heteroatom, the double bond cannot be alpha to the heteroatom. Alkenyl groups may be substituted further with one or more substituents selected from alkyl, alkynyl, alkoxy, cycloalkyl, cycloalkenyl, acyl, acylamino, acyloxy, —NHC(═O)R_{f}, —NR_{f}R_{q}, —C(═O)NR_{f}R_{q}, —NHC(═O)NR_{f}R_{q}, —O—C(═O)NR_{f}R_{q} (wherein R_{f} and R_{q} are the same as defined earlier), alkoxycarbonylamino, azido, cyano, halogen, hydroxy, oxo, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, nitro, or SO₂R₆ (wherein R₆ are is same as defined earlier). Unless otherwise constrained by the definition, alkenyl substituents optionally may be substituted further by 1-3 substituents selected from alkyl, carboxy, hydroxy, alkoxy, halogen, —CF₃, cyano, —NR_{f}R_{q}, —C(═O)NR_{f}R_{q}, —O—C(═O)NR_{f}R_{q} (wherein R_{f} and R_{q} are the same as defined earlier) and —SO₂R₆ (where R₆ is same as defined earlier).

The term “alkynyl,” unless otherwise specified, refers to a monoradical of an unsaturated hydrocarbon, having from 2 to 20 carbon atoms. In the event that alkynyl is attached to a heteroatom, the triple bond cannot be alpha to the heteroatom. Alkynyl groups may be substituted further with one or more substituents selected from alkyl, alkenyl, alkoxy, cycloalkyl, cycloalkenyl, acyl, acylamino, acyloxy, alkoxycarbonylamino, azido, cyano, halogen, hydroxy, oxo, thiocarbonyl, carboxy, arylthio, thiol, alkylthio, aryl, aralkyl, aryloxy, aminosulfonyl, aminocarbonylamino, nitro, heterocyclyl, heteroaryl, heterocyclylalkyl, heteroarylalkyl, —NHC(═O)R_{f}, —NR_{f}R_{q}, —NHC(═O)NR_{f}R_{q}, —C(═O)NR_{f}R_{q}, —O—C(═O)NR_{f}R_{q} (wherein R_{f} and R_{q} are the same as defined earlier), or —SO₂R₆ (wherein R₆ is as defined earlier). Unless otherwise constrained by the definition, alkynyl substituents optionally may be substituted further by 1-3 substituents selected from alkyl, carboxy, carboxyalkyl, hydroxy, alkoxy, halogen, CF₃, —NR_{f}R_{q}, —C(═O)NR_{f}R_{q}, —NHC(═O)NR_{f}R_{q}, C(═O)NR_{f}R_{q} (wherein R_{f} and R_{q} are the same as defined earlier), cyano, or —SO₂R₆ (where R₆ is same as defined earlier).

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like, or multiple ring structures, including adamantanyl, and bicyclo[2.2.1]heptane, or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoky, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR_{f}R_{q}, —NHC(═O)NR_{f}R_{q}, —NHC(═O) R_{f}, —C(═O) NR_{f}R_{q}, —O—C(═O)NR_{f}R_{q} (wherein R_{f} and R_{q} are the same as defined earlier), nitro, heterocyclyl, heteroaryl, heterocyclylalkyl, heteroarylalkyl, or SO₂—R₆ (wherein R₆ is same as defined earlier). Unless otherwise constrained by the definition, cycloalkyl substituents optionally may be substituted further by 1-3 substituents selected from alkyl, carboxy, hydroxy, alkoxy, halogen, CF₃, —NR_{f}R_{q}, —C(═O)NR_{f}R_{q}, —NHC(═O)NR_{f}R_{q}, —O—C(═O)NR_{f}R_{q} (wherein R_{f} and R_{q} are the same as defined earlier), cyano or —SO₂R₆ (where R₆ is same as defined earlier).

The term “alkoxy” denotes the group O-alkyl, wherein alkyl is the same as defined above.

The term “aryl,” unless otherwise specified, refers to carbocyclic aromatic groups, for example, phenyl, biphenyl or napthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COORₑ (wherein Rₑ is hydrogen, alkyl, alkenyl, cycloalkyl, aralkyl, heterocyclylalkyl, heteroarylalkyl), NHC(═O)R_{f}, —NR_{f}R_{q}, —C(═O)NR_{f}R_{q}, —NHC(═O)NR_{f}R_{q}, —OC(═O)NR_{f}R_{q} (wherein R_{f} and R_{q} are the same as defined earlier), —SO₂R₆ (wherein R₆ is same as defined earlier), carboxy, heterocyclyl, heteroaryl, heterocyclylalkyl, heteroarylalkyl or amino carbonyl amino. The aryl group optionally may be fused with a cycloalkyl group, wherein the cycloalkyl group may optionally contain heteroatoms selected from O, N or S.

The term “aralkyl,” unless otherwise specified, refers to alkyl-aryl linked through an alkyl portion (wherein alkyl is as defined above) and the alkyl portion contains 1-6 carbon atoms and aryl is as defined below. Examples of aralkyl groups include benzyl, ethylphenyl and the like.

The term “aralkenyl,” unless otherwise specified, refers to alkenyl-aryl linked through alkenyl (wherein alkenyl is as defined above) portion and the alkenyl portion contains 1 to 6 carbon atoms and aryl is as defined below.

The term “aryloxy” denotes the group O-aryl, wherein aryl is as defined above.

The term “carboxy,” as defined herein, refers to —C(═O)OH.

The term “heteroaryl,” unless otherwise specified, refers to an aromatic ring structure containing 5 or 6 ring atoms, or a bicyclic aromatic group having from 8 to 10 ring atoms, with one or more heteroatom(s) independently selected from N, O or S optionally substituted with 1 to 4 substituent(s) selected from halogen (e.g., F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NR_{f}R_{q}, CH═NOH, —(CH₂)_{w}C(═O)R_{g} {wherein w is an integer from 0-4 and R_{g} is hydrogen, hydroxy, OR_{f}, NR_{f}R_{q}, —NHOR_{z}, or —NHOH}, —C(═O)NR_{f}R_{q} and —NHC(═O)NR_{f}R_{q}, —SO₂R₆, —O—C(═O)NR_{f}R_{q}, —O—C(═O)R_{f}, —O—C(═O)OR_{f} (wherein R₆, R_{f} and R_{q} are as defined earlier, and R_{z} is alkyl, cycloalkyl, aryl, heteroaryl, heterocyclyl, heteroarylalkyl or heterocyclylalkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrazinyl, thienyl, isoxazolyl, triazinyl, furanyl, benzofuranyl, indolyl, benzothiazolyl, or benzoxazolyl, and the like.

The term ‘heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, aryl, alkoxy, aralkyl, cyano, nitro, oxo, carboxy, heterocyclyl, heteroaryl, —O—C(═O)R_{f}, —O—C(═O)OR_{f}, —C(═O)NR_{f}R_{q}, SO₂R₆, —O—C(═O)NR_{f}R_{q}, —NHC(═O)NR_{f}R_{q}, —NR_{f}R_{q} (wherein R₆, R_{f} and R_{q} are as defined earlier) or guanidine. Heterocyclyl can optionally include rings having one or more double bonds. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include oxazolidinyl, tetrahydrofuranyl, dihydrofuranyl, dihydropyridinyl, dihydroisoxazolyl, dihydrobenzofuryl, azabicyclohexyl, dihydroindolyl, pyridinyl, isoindole 1,3-dione, piperidinyl or piperazinyl.

“Heteroarylalkyl” refers to alkyl-heteroaryl group linked through alkyl portion, wherein the alkyl and heteroaryl are as defined earlier.

“Heterocyclylalkyl” refers to alkyl-heterocyclyl group linked through alkyl portion, wherein the alkyl and heterocyclyl are as defined earlier.

“Acyl” refers to —C(═O)R″ wherein R″ is selected from hydrogen, alkyl, cycloalkyl, aryl, aralkyl, heteroaryl, heterocyclyl, heteroarylalkyl or heterocyclylalkyl.

“Alkylcarbonyl” refers to —C(═O)R″, wherein R″ is selected from alkyl, cycloalkyl, aryl, aralkyl, heteroaryl, heterocyclyl, heteroarylalkyl or heterocyclylalkyl.

“Alkylcarboxy” refers to C(═O)R″, wherein R″ is selected from alkyl, cycloalkyl, aryl, aralkyl, heteroaryl, heterocyclyl, heteroarylalkyl or heterocyclylalkyl.

“Amine,” unless otherwise specified, refers to —NH₂. “Substituted amine,” unless otherwise specified, refers to —N(Rₖ)₂, wherein each Rₖ independently is selected from hydrogen {provided that both Rₖ groups are not hydrogen (defined as “amino”)}, alkyl, alkenyl, alkynyl, aralkyl, cycloalkyl, aryl, heteroaryl, heterocyclyl, heterocyclylalkyl, heteroarylalkyl, acyl, SO₂R₆ (wherein R₆ is as defined above), —C(═O)NR_{f}R_{q}, NHC(═O)NR_{f}R_{q}, or —NHC(═O)OR_{f} (wherein R_{f} and R_{q} are as defined earlier).

“Thiocarbonyl” refers to —C(═S)H. “Substituted thiocarbonyl” refers to —C(═S)R″, wherein R″ is selected from alkyl, cycloalkyl, aryl, aralkyl, heteroaryl, heterocyclyl, heteroarylalkyl or heterocyclylalkyl, amine or substituted amine.

Unless otherwise constrained by the definition, all substituents optionally may be substituted further by 1-3 substituents selected from alkyl, aralkyl, cycloalkyl, aryl, heteroaryl, heterocyclyl, carboxy, carboxyalkyl, hydroxy, alkoxy, halogen, CF₃, cyano, —C(=T)NR_{f}R_{q}, —O(C═O)NR_{f}R_{q} (wherein R_{f}, R_{q} and T are the same as defined earlier) and —OC(=T)NR_{f}R_{q}, —SO₂R₆ (where R₆ is the same as defined earlier).

The term “leaving group” refers to groups that exhibit or potentially exhibit the properties of being labile under the synthetic conditions and also, of being readily separated from synthetic products under defined conditions. Examples of leaving groups include, but are not limited to, halogen (e.g., F, Cl, Br, I), triflates, tosylate, mesylates, alkoxy, thioalkoxy, or hydroxy radicals and the like.

The term “protecting groups” refers to moieties that prevent chemical reaction at a location of a molecule intended to be left unaffected during chemical modification of such molecule. Unless otherwise specified, protecting groups may be used on groups, such as hydroxy, amino, or carboxy. Examples of protecting groups are found in T. W. Greene and P. G. M. Wuts, “Protective Groups in Organic Synthesis”, 2^{nd} Ed., John Wiley and Sons, New York, N.Y., which is incorporated herein by reference. The species of the carboxylic protecting groups, amino protecting groups or hydroxy protecting groups employed are not critical, as long as the derivatised moieties/moiety is/are stable to conditions of subsequent reactions and can be removed without disrupting the remainder of the molecule.

The term “pharmaceutically acceptable salts” refers to derivatives of compounds that can be modified by forming their corresponding acid or base salts. Examples of pharmaceutically acceptable salts include, but are not limited to, mineral or organic acids salts of basic residues (such as amines), or alkali or organic salts of acidic residues (such as carboxylic acids), and the like.

### DETAILED DESCRIPTION OF THE INVENTION

The compounds provided herein may be prepared by techniques well known in the art and familiar to a practitioner skilled in art. In addition, the compounds provided herein may be prepared by processes as described herein, such processes not being the only means by which the compounds described may be synthesised. Further, the various synthetic steps described herein may be performed in alternate sequences in order to give the desired compounds.

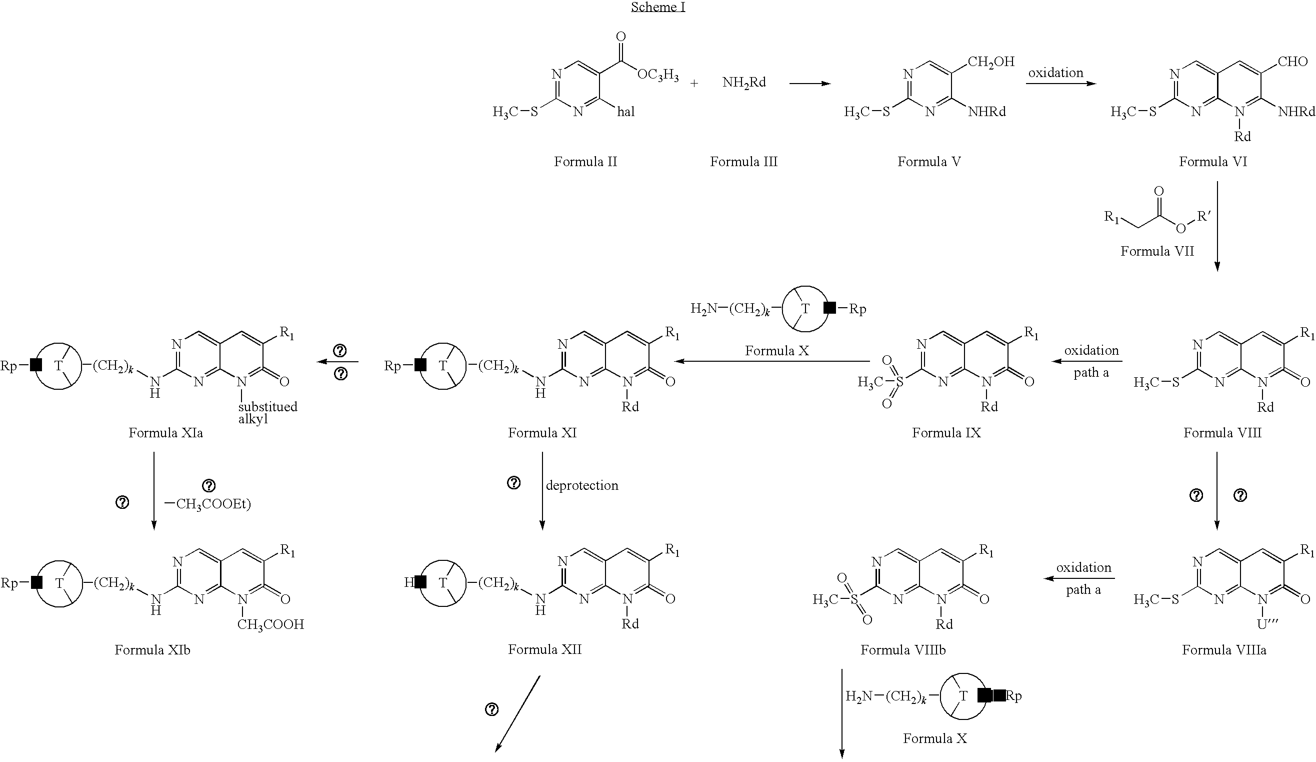

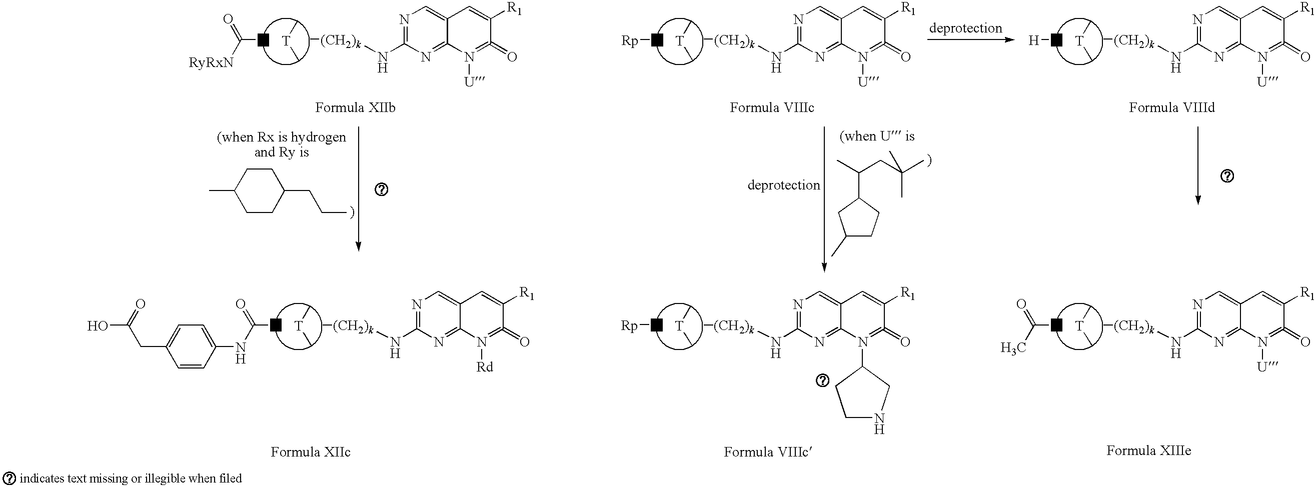

The compounds of Formulae XI, XIa, XIb, VIIIc, VIIIc′, VIIId, VIIIe, XII, XIIb and XIIc may be prepared by following the reaction sequence as depicted in Scheme I. Thus, a compound of Formula II [wherein hal is halogen (Cl, Br or I)] can be reacted with a compound of Formula III (wherein R_{d} is hydrogen, optionally substituted alkyl, cycloalkyl or aryl) to give a compound of Formula IV, which can undergo reduction to give a compound of Formula V, which can be further oxidized to give a compound of Formula VI, which can be reacted with an ester of Formula VII (wherein R′ is alkyl and R₁ is the same as defined earlier) to give a compound of Formula VIII,

Path a: which can be oxidized to give a compound of Formula IX, which can be reacted with a compound of Formula X [wherein Rₚ is alkyl, aralkyl, —C(═O)NRₓR_{y} (wherein Rₓ and Rₓ is the same as defined earlier) or —C(═O)OCH₂C₆H₅ and T & k are the same as defined earlier], to give a compound of Formula XI, which can undergo deprotection (when Rₚ is aralkyl, —C(═O)NRₓR_{y}, —C(═O)OCH₂C₆H₅) to give a compound of Formula XII. The compound of Formula XI can be N-alkylated with a compound of Formula hal-substituted alkyl (wherein hal is Br, Cl, or I) to give a compound of Formula XIa, which can be hydrolyzed to give a compound of Formula XIb. The compound of Formula XII can be reacted with a compound of Formula XIIa (wherein Rₓ and R_{y} are the same as defined earlier) to give a compound of Formula XIIb, which undergoes hydrolysis (when Rₓ is hydrogen and R_{y} is

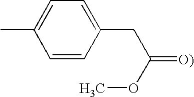

to give a compound of Formula XIIc.
Path b: which can undergo N-derivatization (when R_{d} is hydrogen) with a compound of Formula U″ (wherein U″ is hal-substituted alkyl, —OH(CH₂)ₖN(diethyl),

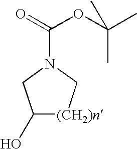

wherein n′ is an integer from 1-2, —OH(CH₂)ₖmorpholine wherein k is the same as defined earlier or

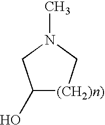

to give a compound of Formula VIIIa (wherein U′″ is substituted alkyl, —(CH₂)ₖN(diethyl),

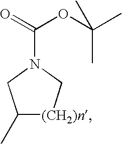

—(CH₂)ₖmorpholine or

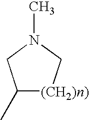

which can be oxidized to give a compound of Formula VIIIb, which can be reacted with a compound of Formula X to give a compound of Formula VIIIc, which can be deprotected to give a compound of Formula VIIId, which can be reacted with acetic anhydride to give a compound of Formula VIIIe.

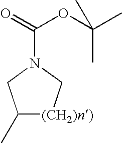

The compound of Formula VIIIc can undergo deprotection (when U′″ is to give a compound of Formula VIIIc′.

The reaction of a compound of Formula II with a compound of Formula III to give a compound of Formula IV can be carried out in an organic solvent, for example, tetrahydrofuran, dimethylformamide, dioxane or diethyl ether in the presence of a base, for example, triethylamine, N-ethyldiisopropylamine, N-methylmorpholine or pyridine.

The compound of Formula IV can be reduced to give a compound of Formula V in an organic solvent, for example, tetrahydrofuran, dimethylformamide, dioxane or diethylether, with reducing agent, for example, lithium aluminium hydride, lithium borohydride, sodium cyanoborohydride or sodium borohydride.

The oxidation of a compound of Formula V to give a compound of Formula VI can be carried out in an organic solvent, for example, dichloromethane, dichloroethane, carbon tetrachloride or chloroform, with an oxidizing agent, for example, manganese dioxide, potassium permanganate, Dess Martin periodinane (DMP), pyridinium dichromate (PDC), pyridinium chlorochromate (PCC) or chromic anhydride, although numerous other methods can be employed (see, for example, Advanced Organic Chemistry, 4^{th} Edn., March, John Wiley & Sons, 1992).

The reaction of a compound of Formula VI with a compound of Formula VII to give a compound of Formula VIII can be carried out in an organic solvent, for example, N-methylpyrrolidinone, dimethylformamide, tetrahydrofuran, diethylether or dioxane, in the presence of a base, for example, potassium carbonate, sodium carbonate, cesium carbonate, potassium carbonate, lithium carbonate or sodium carbonate.

The oxidation of a compound of Formula VIII (path a) to give a compound of Formula IX can be carried out with m-chloroperbenzoic acid or oxone (KHSO₅), in an organic solvent, for example, chloroform, carbon tetrachloride, dichloromethane, dichloroethane, ethanol or tetrahydrofuran.

The reaction of a compound of Formula IX with a compound of Formula X to give a compound of Formula XI can be carried in the presence of a base, for example, pyridine, N-methylmorpholine, N-ethyldiisopropylamine or triethylamine.

Alternatively, in some cases rather than using a compound of Formula IX, a compound of Formula VIII can be reacted directly with a compound of Formula X to give a compound of Formula XI.

The deprotection of a compound of Formula XI (when Rₚ is —C(═O)OCH₂C₆H₅) to give a compound of Formula XII can be carried out in an organic solvent, for example, methanol, ethanol, propanol or isopropylalcohol, in the presence of a base, for example potassium hydroxide, sodium hydroxide or lithium hydroxide.

The N-alkylation of a compound of Formula XI (when R_{d} is hydrogen) can be carried out with hal-substituted alkyl to give a compound of Formula XIa in an organic solvent for example, N-methylpyrrolidone, dimethylformamide, tetrahydrofuran, diethylether or dioxane in the presence of a base for example, sodium hydride, N-butyllithium, potassium tert-butoxide or diisopropylethylamine.

The hydrolysis of a compound of Formula XIa can be carried out in the presence of a base for example, sodium hydroxide, lithium hydroxide or potassium hydroxide in the presence of an alcohol for example, methanol, ethanol, propanol or isopropylalcohol.

The compound of Formula XII can undergo reaction with a compound of Formula XIIa to give a compound of Formula XIIb in the presence of a base for example, Hunig's base, sodium hydride, potassium tert-butoxide in an organic solvent for example, dimethyl-sulphoxide, dimethylformamide, tetrahydrofuran, diethylether or dioxane.

The hydrolysis of a compound of Formula XIIIb can be carried out in the presence of base for example, sodium hydroxide, lithium hydroxide or potassium hydroxide in an organic solvent for example, methanol, ethanol, propanol or isopropylalcohol.

The N-derivatization of a compound of Formula VIII (when R_{d} is hydrogen) (path b) can be carried out with a compound of Formula U″ (when U″ is hal-substituted alkyl) to give a compound of Formula VIIa can be carried out in an organic solvent for example, N-methylpyrrolidone, dimethylformamide, tetrahydrofuran, diethylether or dioxane in the presence of a base for example, sodium hydride, potassium tert-butoxide, N-butyllithium, or diisopropylethylamine.

The N-derivatization of a compound of Formula VIII (when R_{d} is hydrogen) (path b) can be carried out with a compound of Formula U″ (when U″ is —OH(CH₂)ₖN(diethyl),

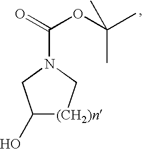

—OH(CH₂)ₖmorpholine or

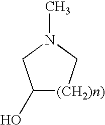

to give a compound of Formula VIIIa can be carried out in an organic solvent of example, tetrahydrofuran, diethylether, dioxane, toluene, benzene or dimethylformamide in the presence of a redox couple. The redox couple agents may be any one of those known to a person skilled in the art of organic synthesis. The oxidizing part of the redox couple is for example, diisopropylazodicarboxylate (DIAD), diethylazodicarboxylate (DEAD), N,N,N′N′-tetramethylazodicarboxamide (TMAD), 1,1′-(azodicarbonyl)dipiperidine (ADDP), cyanomethylenetributylphosphorane (CMBP), 4,7-dimethyl-3,5,7-hexahydro-1,2,4,7-tetrazocin-3,8-dione (DHTD) or N,N,N′N′-tetraisopropylazodicarboxamide (TIPA). The reduction part of the redox couple is a phosphine for example, trialkylphosphine (such as tributylphosphine), triarylphosphine (such as triphenylphosphine), tricycloalkylphosphine (such as tricyclohexylphosphine) or triheteroarylphosphine. The phosphine reagent with a combination of aryl, alkyl or heteroaryl substituents may also be used (such as diphenylpyridylphosphine).

The oxidation of a compound of Formula VIIIa to give a compound of Formula VIIIb can be carried out with m-chloroperbenzoic acid or oxone (KHSO₅), in an organic solvent, for example, chloroform, carbon tetrachloride, dichloromethane, dichloroethane, ethanol or tetrahydrofuran.

The reaction of a compound of Formula VIIIb with a compound of Formula X to give a compound of Formula VIIIc can be carried in the presence of a base, for example, pyridine, N-methylmorpholine, N-ethyldiisopropylamine or triethylamine.

The deprotection of Formula VIIIc (wherein Rₚ can be aralkyl) to give a compound of Formula VIIId can be carried out in an organic solvent (for example, methanol, ethanol, propanol or isopropylalcohol) in the presence of a deprotecting agent (for example, palladium on carbon in presence of hydrogen gas or palladium on carbon with a source of hydrogen gas (for example, ammonium formate solution, cyclohexene or formic acid)).

The compound of Formula VIIId can be reacted with acetic anhydride to give a compound of Formula VIIIe can be carried out in the presence of a base, for example, pyridine, N-methylmorpholine, N-ethyldiisopropylamine or triethylamine

The deprotection of a compound of Formula VIIIc to give a compound of Formula VIIIc′ can be carried out in an organic solvent, for example, methanol, ethanol, propanol or isopropylalcohol, in the presence of a base, for example potassium hydroxide, sodium hydroxide or lithium hydroxide.

Particular illustrative compounds include the following:
2-(3-Benzyl-3-aza-bicyclo[3.1.0]hex-6-ylamino)-6-(2-chlorophenyl)-8-methyl-8H-pyrido[2,3-d]pyrimidin-7-one (Compound No. 1);
2-(3-Aza-bicyclo[3.1.0]hex-6-ylamino)-6-(2-chlorophenyl)-8-methyl-8H-pyrido[2,3-d]pyrimidin-7-one (Compound No. 5);
6-[6-(2-Chlorophenyl)-8-methyl-7-oxo-7,8-dihydro-pyrido[2,3-d]pyrimidin-2-ylamino]-3-aza-bicyclo[3.1.0]hexane-3-carboxylic acid benzyl ester (Compound No. 6);
2-(3-Benzyl-3-aza-bicyclo[3.2.1]oct-8-ylamino)-6-(2-chlorophenyl)-8-methyl-8H-pyrido[2,3-d]pyrimidin-7-one (Compound No. 7);
2-(3-Benzyl-3-aza-bicyclo[3.1.0]hex-6-ylamino)-6-(2-chlorophenyl)-8-(4-fluorophenyl)-8H-pyrido[2,3-d]pyrimidin-7-one (Compound No. 8);
2-(3-Benzyl-3-aza-bicyclo[3.2.1]oct-8-ylamino)-6-(2-chlorophenyl)-8-(4-fluorophenyl)-8H-pyrido[2,3-d]pyrimidin-7-one (Compound No. 9);
6-[6-(2-Chlorophenyl)-8-(4-fluorophenyl)-7-oxo-7,8-dihydro-pyrido[2,3-d]pyrimidin-2-ylamino]-3-aza-bicyclo[3.1.0]hexane-3-carboxylic acid (4-fluorophenyl)-amide (Compound No. 10);
6-[6-(2-Chlorophenyl)-8-(4-fluorophenyl)-7-oxo-7,8-dihydro-pyrido[2,3-d]pyrimidin-2-ylamino]-3-aza-bicyclo[3.1.0]hexane-3-carboxylic acid benzyl ester (Compound No. 12);
2-(3-Aza-bicyclo[3.1.0]hex-6-ylamino)-6-(2-chlorophenyl)-8-(4-fluorophenyl)-8H-pyrido[2,3-d]pyrimidin-7-one (Compound No. 13);
6-[6-(2-Chlorophenyl)-8-(4-fluorophenyl)-7-oxo-7,8-dihydro-pyrido[2,3-d]pyrimidin-2-ylamino]-3-aza-bicyclo[3.1.0]hexane-3-carboxylic acid isopropylamide (Compound No. 14);
2-[(3-Benzyl-3-aza-bicyclo[3.1.0]hex-6-ylmethyl)-amino]-6-(2-chlorophenyl)-8-(4-fluorophenyl)-8H-pyrido[2,3-d]pyrimidin-7-one (Compound No. 16);
2-(3-Benzyl-3-aza-bicyclo[3.1.0]hex-6-ylamino)-6-(2-chlorophenyl)-8-cyclopropyl-8H-pyrido[2,3-d]pyrimidin-7-one (Compound No. 17);
6-[6-(2-Chlorophenyl)-8-cyclopropyl-7-oxo-7,8-dihydro-pyrido[2,3-d]pyrimidin-2-ylamino]-3-aza-bicyclo[3.1.0]hexane-3-carboxylic acid benzyl ester (Compound No.
18);
2-(3-Aza-bicyclo[3.1.0]hex-6-ylamino)-6-(2-chlorophenyl)-8-cyclopropyl-8H-pyrido[2,3-d]pyrimidin-7-one (Compound No. 19);
6-(2-Chlorophenyl)-8-cyclopropyl-2-(3-methyl-3-aza-bicyclo[3.1.0]hex-6-ylamino)-8H-pyrido[2,3-d]pyrimidin-7-one (Compound No. 20);
6-[6-(2-Chlorophenyl)-8-cyclopropyl-7-oxo-7,8-dihydro-pyrido[2,3-d]pyrimidin-2-ylamino]-3-aza-bicyclo[3.1.0]hexane-3-carboxylic acid-(4-fluorophenyl)-amide (Compound No. 26).
2-(3-Benzyl-3-aza-bicyclo[3.1.0]hex-6-ylamino)-6-(2-chlorophenyl)-8H-pyrido[2,3-d]pyrimidin-7-one (Compound No. 27);
2-(1-Benzylpiperidin-4-ylamino)-6-(2-chlorophenyl)-8-cyclopropyl-8H-pyrido[2,3-d]pyrimidin-7-one (Compound No. 32);
2-(7-Benzyl-3-oxa-7-aza-bicyclo[3.3.1]non-9-ylamino)-6-(2-chlorophenyl)-8-cyclopropyl-8H-pyrido[2,3-d]pyrimidin-7-one (Compound No. 33);
2-(9-Benzyl-9-aza-bicyclo[3.3.1]non-3-ylamino)-6-(2-chlorophenyl)-8-cyclopropyl-8H-pyrido[2,3-d]pyrimidin-7-one (Compound No. 34);
2-(3-Benzyl-3-aza-bicyclo[3.1.0]hex-6-ylamino)-6-(2-chlorophenyl)-8-cyclopropyl-8H-pyrido[2,3-d]pyrimidin-7-one (Compound No. 35);
2-(9-Benzyl-9-aza-bicyclo[3.3.1]non-3-ylamino)-6-(2-chlorophenyl)-8-(4-fluorophenyl)-8H-pyrido[2,3-d]pyrimidin-7-one (Compound No. 50);
2-(9-Benzyl-9-aza-bicyclo[3.3.1]non-3-ylamino)-6-(2-chlorophenyl)-8-methyl-8H-pyrido[2,3-d]pyrimidin-7-one (Compound No. 51);
2-(7-Benzyl-3-oxa-7-aza-bicyclo[3.3.1]non-9-ylamino)-6-(2-chlorophenyl)-8-(4-fluorophenyl)-8H-pyrido[2,3-d]pyrimidin-7-one (Compound No. 52);
2-(7-Benzyl-3-oxa-7-aza-bicyclo[3.3.1]non-9-ylamino)-6-(2-chlorophenyl)-8-methyl-8H-pyrido[2,3-d]pyrimidin-7-one (Compound No. 53);
2-(8-Benzyl-8-aza-bicyclo[3.2.1]oct-3-ylamino)-6-(2-chlorophenyl)-8-(4-fluorophenyl)-8H-pyrido[2,3-d]pyrimidin-7-one (Compound No. 54);
2-(8-Benzyl-8-aza-bicyclo[3.2.1]oct-3-ylamino)-6-(2-chlorophenyl)-8-methyl-8H-pyrido[2,3-d]pyrimidin-7-one (Compound No. 55);
[2-(3-Benzyl-3-aza-bicycyclo[3.1.0]hex-6-ylamino)-6-(2-chlorophenyl)-7-oxo-7H-pyrido[2,3-d]pyrimidin-8-yl]-acetonitrile. (Compound No. 56);
6-[6-(2-Chlorophenyl)-8-cyanomethyl-7-oxo-7,8-dihydro-pyrido[2,3-d]pyrimidin-2-ylamino]-3-aza-bicyclo[3.1.0]hexane-3-carboxylic acid (4-fluorophenyl)-amide (Compound No. 57);
2-(3-Aza-bicyclo[3.1.0]hex-6-ylamino)-6-(2-chlorophenyl)-8H-pyrido[2,3-d]pyrimidin-7-one. (Compound No. 58);
2-(8-Benzyl-8-aza-bicyclo[3.2.1]oct-3-ylamino)-6-(2-chlorophenyl)-8H-pyrido[2,3-d]pyrimidin-7-one (Compound No. 59);
2-(7-Benzyl-3-oxa-7-aza-bicyclo[3.3.1]non-9-ylamino}-6-(2-chlorophenyl)-8H-pyrido[2,3-d]pyrimidin-7-one (Compound No. 60);
2-(7-Benzyl-3-oxa-7-aza-bicyclo[3.3.1]non-9-ylamino)-6-(2-chlorophenyl)-8H-pyrido[2,3-d]pyrimidin-7-one (Compound No. 61);
9-[6-(2-chlorophenyl)-8-cyclopropyl-7-oxo-7,8-dihydro-pyrido[2,3-d]pyrimidin-2-ylamino]-3-oxa-7-aza-bicyclo[3.3.1]nonane-7-carboxylic acid benzyl ester (Compound No. 65);
6-[6-(2-Chlorophenyl)-8-cyclopropyl-7-oxo-7,8-dihydro-pyrido[2,3-d]pyrimidin-2-ylamino]-3-aza-bicyclo[3.1.0]hexane-3-carboxylic acid ethyl ester (Compound No. 66);
6-[6-(2-Chlorophenyl)-8-methyl-7-oxo-7,8-dihydro-pyrido[2,-d]pyrimidin-2-ylamino]-3-aza-bicyclo[3.1.0]hexane-3-carboxylic acid ethyl ester (Compound No. 68);
6-[6-(2-Chlorophenyl)-7-oxo-7,8-dihydro-pyrido[2,3-d]pyrimidin-2-ylamino]-3-aza-bicyclo[3.1.0]hexane-3-carboxylic acid benzyl ester (Compound No. 69);
2-(3-Acetyl-3-aza-bicyclo[3.1.0]hex-6-ylamino)-6-(2-chlorophenyl)-8-(2-diethylaminoethyl)-8H-pyrido[2,3-d]pyrimidin-7-one (Compound No. 70);
3-[2-(3-Benzyl-3-aza-bicyclo[3.1.0]hex-6-ylamino)-6-(2-chlorophenyl)-7-oxo-7H-pyrido[2,3-d]pyrimidin-8-yl]-pyrrolidine-1-carboxylic acid tert-butyl ester (Compound No. 71);
2-(3-Benzyl-3-aza-bicyclo[3.1.0]hex-6-ylamino)-6-(2-chlorophenyl)-8-(2-morpholin-4-yl-ethyl)-8H-pyrido[2,3-d]pyrimidin-7-one (Compound No. 72);
2-(3-Benzyl-3-aza-bicyclo[3.1.0]hex-6-ylamino)-6-(2-chlorophenyl)-8-(2-diethylaminoethyl)-8H-pyrido[2,3-d]pyrimidin-7-one (Compound No. 73);
2-(3-Benzyl-3-aza-bicyclo[3.1.0]hex-6-ylamino}-6-(2-chlorophenyl)-8-(1-methyl-pyrrolidin-3-yl)-8H-pyrido[2,3-d]pyrimidin-7-one (Compound No. 74);
[2-(3-Benzyl-3-aza-bicyclo[3.1.0]hex-6-ylamino)-6-(2-chlorophenyl)-7-oxo-7H-pyrido[2,3-d]pyrimidin-8-yl]-acetic acid ethyl ester (Compound No. 75);
[2-(3-Benzyl-3-aza-bicyclo[3.1.0]hex-6-ylamino)-6-(2-chlorophenyl)-7-oxo-7H-pyrido[2,3-d]pyrimidin-8-yl]-acetic acid (Compound No. 76);
2-(3-Benzyl-3-aza-bicyclo[3.1.0]hex-6-ylamino)-6-(2-chlorophenyl)-8-pyrrolidin-3-yl-8H-pyrido[2,3-d]pyrimidin-7-one (Compound No. 77);
2-[2-(3-Benzyl-3-aza-bicyclo[3.1.0]hex-6-ylamino)-6-(2-chlorophenyl)-7-oxo-7H-pyrido[2,3-d]pyrimidin-8-yl]-acetamide (Compound No. 79);
[4-{6-[6-(2-Chlorophenyl)-8-methyl-7-oxo-7,8-dihydro-pyrido[2,3-d{pyrimidin-2-ylamino]-3-aza-bicyclo[3.1.0]hexane-3-carbonyl]-amino}-phenyl]-acetic acid (Compound No. 80);
[4-({6-[6-(2-Chlorophenyl)-8-methyl-7-oxo-7,8-dihydro-pyrido[2,3-d]pyrimidin-2-ylamino]-3-aza-bicyclo[3.1.0]hexane-3-carbonyl]-amino)-phenyl]-acetic acid (Compound No. 81);

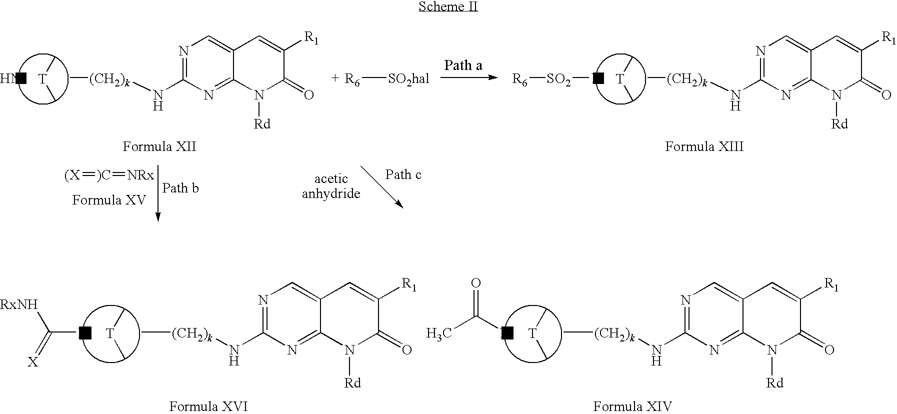

The compounds of Formulae XIII, XIV and XVI may be prepared for example, by reaction sequences as depicted in Scheme II,

Path a: the compound of Formula XII can be reacted with R₆—SO₂-hal (wherein hal is halogen (Cl, Br, I) and R₆ is the same as defined earlier) to give a compound of Formula XIII.

Path b: the compound of Formula XII can be reacted with a compound of Formula XV (wherein X is oxygen or sulphur and Rₓ is the same as defined earlier) to give a compound of Formula XVI.

Path c: the compound of Formula XII is reacted with acetic anhydride to give a compound of Formula XIV.

The reaction of a compound of Formula XII (Path a) with R₆—SO₂-hal to give a compound of Formula XIII can be carried out in an organic solvent, for example, dichloromethane, dichloroethane, carbon tetrachloride or chloroform, in the presence of a base, for example, triethylamine, N-ethyldiisopropylamine, N-methylmorpholine or pyridine.

The reaction of a compound of Formula XII with a compound of Formula XV (Path b) to give a compound of Formula XVI can be carried out in an organic solvent, for example, dichloromethane, dichloroethane, carbon tetrachloride or chloroform, in the presence of a base, for example, triethylamine, N-ethyldiisopropylamine or N-methylmorpholine.

The reaction of a compound of Formula XII with acetic anhydride (Path c) to give a compound of Formula XIV can be carried-out in the presence of a base, for example, pyridine, N-methylmorpholine or N-ethyldiisopropylamine.

Particular illustrative compounds include the following:
6-[6-(2-Chlorophenyl)-8-methyl-7-oxo-7,8-dihydro-pyrido[2,3-d]pyrimidin-2-ylamino]-3-aza-bicyclo[3.1.0]hexane-3-carboxylic acid-(4-fluorophenyl)-amide (Compound No. 2);
6-[6-(2-Chlorophenyl)-8-methyl-7-oxo-7,8-dihydro-pyrido[2,3-d]pyrimidin-2-ylamino]-3-aza-bicyclo[3.1.0]hexane-3-carboxylic acid isopropylamide (Compound No. 3);
6-(2-Chlorophenyl)-2-(3-methanesulphonyl-3-aza-bicyclo[3.1.0]hex-6-ylamino)-8-methyl-8H-pyrido[2,3-d]pyrimidin-7-one (Compound No. 4);
6-(2-Chlorophenyl)-8-(4-fluorophenyl)-2-(3-methanesulphonyl-3-aza-bicyclo[3.1.0]hex-6-ylamino)-8H-pyrido[2,3-d]pyrimidin-7-one (Compound No. 11);
6-(2-Chlorophenyl)-8-(4-fluorophenyl)-2-[3-(toluene-4-sulphonyl)-3-aza-bicyclo[3.1.0]hex-6-ylamino]-8H-pyrido[2,3-d]pyrimidin-7-one (Compound No. 15);
6-(2-Chlorophenyl)-8-cyclopropyl-2-[3-(toluene-4-sulphonyl)-3-aza-bicyclo[3.1.0]hex-6-ylamino]-8H-pyrido[2,3-d]pyrimidin-7-one (Compound No. 21);
6-(2-Chlorophenyl)-8-cyclopropyl-2-(3-methanesulphonyl-3-aza-bicyclo[3.1.0]hex-6-ylamino)-8H-pyrido[2,3-d]pyrimidin-7-one (Compound No. 22);
2-(3-Acetyl-3-aza-bicyclo[3.1.0]hex-6-ylamino)-6-(2-chlorophenyl)-8-cyclopropyl-8H-pyrido[2,3-d]pyrimidin-7-one (Compound No. 23);
6-(2-Chlorophenyl)-8-cyclopropyl-2-{3-[2-(1,1,3,3-tetramethylbutylamino)-acetyl]-3-aza-bicyclo[3.1.0]hex-6-ylamino}-8H-pyrido[2,3-d]pyrimidin-7-one (Compound No. 24).
6-[6-(2-Chlorophenyl)-8-cyclopropyl-7-oxo-7,8-dihydro-pyrido[2,3-d]pyrimidin-2-ylamino]-3-aza-bicyclo[3.1.0]hexane-3-carboxylic acid isopropylamide (Compound No. 29);
6-[6-(2-Chlorophenyl)-8-cyclopropyl-7-oxo-7,8-dihydro-pyrido[2,3-d]pyrimidin-2-ylamino]-3-aza-bicyclo[3.1.0]hexane-3-carbothioic acid isopropylamide (Compound No. 30)
2-(3-Benzenesulphonyl-3-aza-bicyclo[3.1.0]hex-6-ylamino)-6-(2-chlorophenyl)-8-cyclopropyl-8H-pyrido[2,3-d]pyrimidin-7-one (Compound No. 31)
6-(2-Chlorophenyl)-8-cyclopropyl-2-[3-(thiophene-2-sulphonyl)-3-aza-bicyclo[3.1.0]hex-6-ylamino]-8H-pyrido[2,3-d]pyrimidin-7-one (Compound No. 36);
6-(2-Chlorophenyl)-8-cyclopropyl-2-[3-(4-trifluoromethylbenzenesulfonyl)-3-aza-bicyclo[3.1.0]hex-6-ylamino]-8H-pyrido[2,3-d]pyrimidin-7-one (Compound No. 37);
6-(2-Chlorophenyl)-8-cyclopropyl-2-[3-(4-ethoxybenzenesulfonyl)-3-aza-bicyclo[3.1.0]hex-6-ylamino]-8H-pyrido[2,3-d]pyrimidin-7-one (Compound No. 38);
6-(2-Chlorophenyl)-8-cyclopropyl-2-(3-ethanesulfonyl-3-aza-bicyclo[3.1.0]hex-6-ylamino)-8H-pyrido[2,3-d]pyrimidin-7-one (Compound No. 39);
6-(2-Chlorophenyl-8-methyl-2-[3-(thiophene-2-sulfonyl)-3-aza-bicyclo[3.1.0]hex-6-ylamino]-8H-pyrido[2,3-d]pyrimidin-7-one (Compound No. 40);
2-(3-Benzenesulphonyl-3-aza-bicyclo[3.1.0]hex-6-ylamino)-6-(2-chlorophenyl)-8-methyl-8H-pyrido[2,3-d]pyrimidin-7-one (Compound No. 41);
6-(2-Chlorophenyl)-2-(3-ethanesulfonyl-3-aza-bicyclo[3.1.0]hex-6-ylamino)-8-methyl-8H-pyrido[2,3-d]pyrimidin-7-one (Compound No. 42);
6-(2-Chlorophenyl)-8-methyl-2-[3-(toluene-4-sulfonyl)-3-aza-bicyclo[3.1.0]hex-6-ylamino]-8H-pyrido[2,3-d]pyrimidin-7-one (Compound No. 43);
2-(3-Acetyl-3-aza-bicyclo[3.1.0]hex-6-ylamino)-6-(2-chlorophenyl)-8-methyl-8H-pyrido[2,3-d]pyrimidin-7-one (Compound No. 45);
6-[6-(2-Chlorophenyl)-8-methyl-7-oxo-7,8-dihydro-pyrido[2,3-d]pyrimidin-2-ylamino]-3-aza-bicyclo[3.1.0]hexane-3-carboxylic acid (1,1,3,3-tetramethylbutyl)-amide (Compound No. 46);
6-(2-Chlorophenyl)-2-[3-(4-methoxybenzenesulfonyl)-3-aza-bicyclo[3.1.0]hex-6-ylamino]-8-methyl-8H-pyrido[2,3-d]pyrimidin-7-one (Compound No. 47);
2-(3-Acetyl-3-aza-bicyclo[3.1.0]hex-6-ylamino)-6-(2-chlorophenyl)-8H-pyrido[2,3-d]pyrimidin-7-one (Compound No. 48);
2-(3-Acetyl-3-aza-bicyclo[3.1.0]hex-ylamine)-6-(2-chlorophenyl)-8H-pyrido[2,3-]pyrimidin-7-one (Compound No. 78).

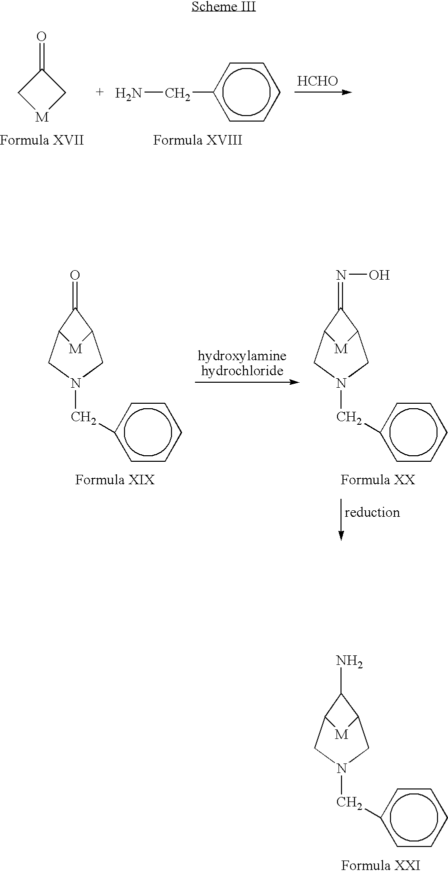

The compound of Formula XXI, can be prepared for example, by reaction sequences as depicted in Scheme III. Thus a compound of Formula XVII (wherein M is —CH₂, —CH₂CH₂—, —CH(O)CH₂, —CH(O) or —CH₂OCH₂) can be reacted with a compound of Formula XVIII and with formaldehyde to give a compound of Formula XIX, which can be reacted with hydroxylamine hydrochloride to give a compound of Formula XX, which can be reduced to give a compound of Formula XXI.

The reaction of a compound of Formula XVII with a compound of Formula XVIII with formaldehyde to give a compound of Formula XIX can be carried out in an organic solvent, for example, methanol, ethanol, propanol or isopropylalcohol.

The compound of Formula XIX is reacted with hydroxylamine hydrochloride to give a compound of Formula XX in solvent mixture, for example, ethanol in water or methanol in water, in the presence of a base, for example, sodium bicarbonate, potassium bicarbonate, lithium bicarbonate or sodium acetate.

The reduction of a compound of Formula XX to give a compound of Formula XXI can be carried out in an organic solvent, for example, tetrahydrofuran, dimethylformamide, dioxane or diethylether, with reducing agent, for example, lithium aluminium hydride, sodium borohydride, sodium cyanoborohydride or lithium borohydride. Particular compounds are described below:
3-benzyl-8-amino-3-aza-bicyclo[3.2.1]octane

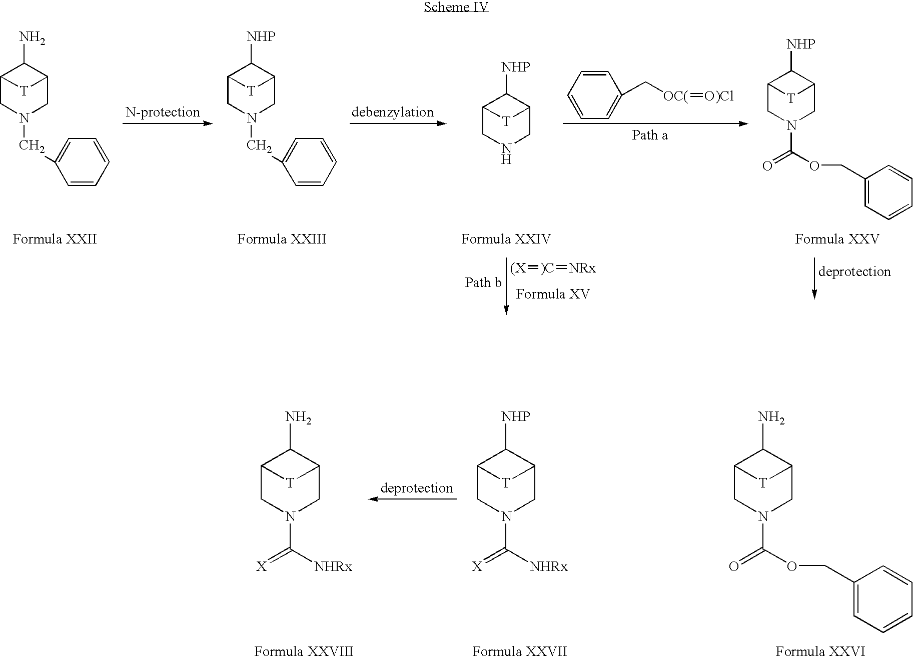

The compound of Formula XXVI and XXVIII may be prepared by reaction sequences as depicted in Scheme IV, thus a compound of Formula XXII (wherein T is the same as defined earlier) can undergo N-protection to give a compound of Formula XXIII [(wherein P is —C(═O)OC(CH₃)₃ (BOC), —C(═O)OC(CH₃)₂CHBr₂ (DB t-BOC) or —C(═O) OC(CH₃)₂CCl₃ (TC BOC)] which can undergo debenzylation to give a compound of Formula XXIV.

Path a: The compound of Formula XXIV can be reacted with benzyl chloroformate to give a compound of Formula XXV, which can be deprotected to give a compound of Formula XXVI.

Path b: The compound of Formula XXIV can be reacted with a compound of Formula XV to give a compound of Formula XXVII, which can be deprotected to give a compound of Formula XXVIII.

The compound of Formula XXII can be protected to give a compound of Formula XXIII [when P is —C(═O)OC(CH₃)₃ (BOC)] with di-tert-butyl dicarbonate in an organic solvent, for example, dichloromethane, dichloroethane, carbon tetrachloride or chloroform, in the presence of a base, for example, sodium hydroxide, sodium bicarbonate, triethylamine or N-ethyldiisopropylamine.

Alternatively, when P is —C(═O)OC(CH₃)₂CHBr₂ (DB t-BOC) or —C(═O)OC(CH₃)₂CCl₃ (TC-BOC), the compound of Formula XXII can be protected to give a compound of Formula XXIII by following procedures as described in, for example, T. W. Greene and P. G. M. Wuts, “Protective Groups in Organic Synthesis”, 2^{nd} Edn., John Wiley and Sons, New York.

The compound of Formula XXIII can be debenzylated to give a compound of Formula XXIV in an organic solvent, for example, methanol, ethanol, propanol or isopropylalcohol, in the presence of a catalyst, for example, palladium on carbon and ammonium formate.

The compound of Formula XXIV (Path a) can be reacted with benzyl chloroformate to give a compound of Formula XXV in an organic solvent, for example dichloromethane, dichloroethane, carbon tetrachloride or chloroform, in the presence of a base, for example, triethylamine, N-ethyldiisopropylamine, N-methylmorpholine or pyridine.

The deprotection of a compound of Formula XXV [(when P is —C(═O)OC(CH₃)₃(BOC)] to give a compound of Formula XXVI can be carried out in an organic solvent, for example, methanol, ethanol, propanol or isopropyl alcohol, in the presence of ethanolic or methanolic hydrochloric acid or trifluoroacetic acid.

The deprotection of a compound of Formula XXV (when P is —C(═O)OC(CH₃)₂CBr₂ (DB-t-BOC) to give a compound of Formula XXVI can be carried out in an organic solvent, for example, ethanol, methanol, propanol or isopropylalcohol or by hydrobromic acid (45% w/v solution in acetic acid).

The deprotection of a compound of Formula XXV [when P is —C(═O)OC(CH₃)₂CCl₃ (TC-BOC)] to give a compound of Formula XXVI can be carried out by treatment with a super-nucleophile, for example, lithium cobalt(I) phthalocyanine, zinc and acetic acid or cobalt phthalocyanine.

The compound of Formula XXIV (Path b) can be reacted with a compound of Formula XV to give a compound of Formula XXVII in an organic solvent, for example, dichloroethane, dichloromethane, carbon tetrachloride or chloroform.

The deprotection of a compound of Formula XXVII to give a compound of Formula XXVIII can be carried out following, for example, procedures as described in the synthesis of compound of Formula XXVI from a compound of Formula XXV.

Particular illustrative compounds prepared through Scheme IV include the following:
6-Amino-3-aza-bicyclo[3.1.0]hexane-3-carboxylic acid benzyl ester
6-Amino-3-aza-bicyclo[3.1.0]hexane-3-carboxylic acid-(4-fluorophenyl)-amide
6-Amino-3-azabicyclo[3.1.0]hexane-3-carboxylic acid isopropylamide

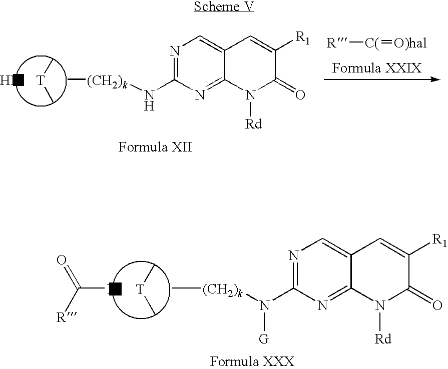

The compound of Formula XXX can be prepared by following, for example, the reaction sequence as depicted in Scheme V. Thus a compound of Formula XII may be reacted with a compound of Formula XXIX (wherein R′″ is alkyl or aryl) to give a compound of Formula XXX (wherein G is H or —C(═O)R′″). As is apparent from the compound of Formula XII, there are two secondary amine groups, so that reaction with R′″—C(═O)hal can result in either mono-derivatization (wherein G remains H) or bis-derivatization (wherein G is —C(═O)R′″).

The reaction of a compound of Formula XII with a compound of Formula XXIX to give a compound of Formula XXX can be carried out in the presence of a base, for example, pyridine, N-methylmorpholine, N-ethyldiisopropylamine or triethylamine.

Particular illustrative compounds include the following:
N-(3-Benzoyl-3-aza-bicyclo[3.1.0]hex-6-yl)-N-[6-(2-chlorophenyl)-8-cyclopropyl-7-oxo-7,8-dihydro-pyrido[2,3-d]pyrimidin-2-yl]-benzamide (Compound No. 25);
2-(3-Benzoyl-3-aza-bicyclo[3.1.0]hex-6-ylamino)-6-(2-chlorophenyl)-8-cyclopropyl-8H-pyrido[2,3-d]pyrimidin-7-one (Compound No. 28);
6-(2-Chlorophenyl)-8-methyl-2-[3-(4-methylbenzoyl)-3-aza-bicyclo[3.1.0]hex-6-ylamino]-8H-pyrido[2,3-d]pyrimidin-7-one (Compound No. 44).

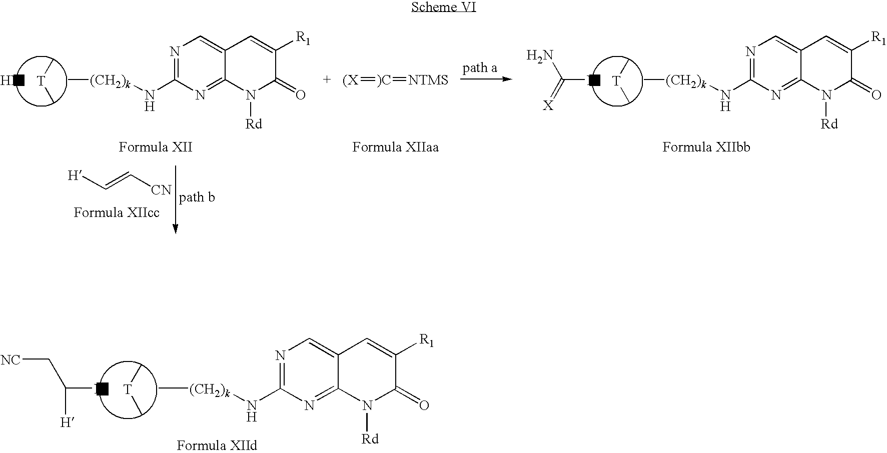

The compounds of Formula XIIbb can be prepared following the procedure as depicted in Scheme VI. The reaction comprises reacting a compound of Formula XII
Path a: with a compound of Formula XIIaa (wherein TMS is trimethylsilane) to give a compound of Formula XIIbb.
Path b: with a compound of Formula XIIcc (wherein H′ is hydrogen, alkyl, cycloalkyl, aryl, heteroaryl or heterocyclyl) to give a compound of Formula XIId.

The reaction of a compound of Formula XII with a compound of Formula XIIaa to give a compound of Formula XIIbb (path a) can be carried out in an organic solvent, for example, dichloromethane, dichloroethane, carbon tetrachloride or chloroform, in the presence of a base, for example, triethylamine, N-ethyldiisopropylamine, N-methylmorpholine or pyridine.

The reaction of a compound of Formula XII with a compound of Formula XIIcc (path b) to give a compound of Formula XIId can be carried out in an organic solvent selected from methanol, ethanol, propanol or isopropyl alcohol.

Particular illustrative compounds include the following:
6-[6-(2-Chlorophenyl)-8-cyclopropyl-7-oxo-7,8-dihydro-pyrido[2,3-d]pyrimidin-2-ylamino]-3-aza-bicyclo[3.1.0]hexane-3-carboxylic acid amide (Compound No. 63);
3-{6-[6-(2-Chlorophenyl)-8-cyclopropyl-7-oxo-7,8-dihydro-pyrido[2,3-d]pyrimidin-2-ylamino]-3-aza-bicyclo[3.1.0]hex-3-yl}-propionitrile (Compound No. 64);
3-{6-[6-(2-Chlorophenyl)-8-methyl-7-oxo-7,8-dihydro-pyrido[2,3-d]pyrimidin-2-ylamino]-3-aza-bicyclo[3.1.0]hex-3-yl}-propionitrile (Compound No. 67)

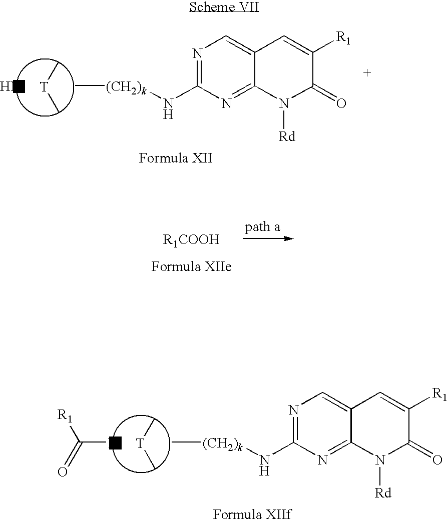

The compound of Formula XIIf can be prepared by following the procedure as depicted in Scheme VII. The reaction comprises reacting a compound of Formula XII (wherein R₁, R_{d} and k are the same as defined earlier) with a compound of Formula XIIe (wherein R₁ is the same as defined earlier) to give a compound of Formula XIIf.

The reaction of a compound of Formula XII with a compound of Formula XIIe to give a compound of Formula XIIf can be carried out in an in an organic solvent (for example, dimethylformamide, dichloromethane, chloroform, tetrahydrofuran, dioxane or diethylether) in presence of a base (for example, N-methylmorpholine, triethylamine, diisopropylethylamine or pyridine) with a condensing agent (for example, 1-(3-dimethylaminopropyl)-3-ethylcarbodiimide hydrochloride (EDCI.HCl) or dicyclohexylcarbodiimide (DCC).

Particular illustrative compounds include the following:
2-(3-Benzoyl-3-aza-bicyclo[3.1.0]hex-6-ylamino)-6-(2-chlorophenyl)-8-(4-fluorophenyl)-8H-pyrido[2,3-d]pyrimidin-7-one (Compound No. 49);

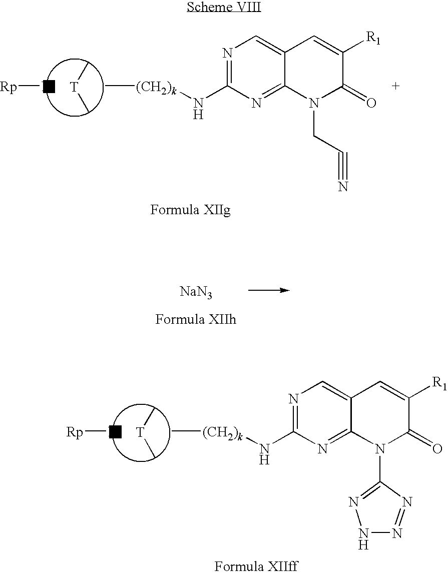

The compounds of Formula XIIff can be prepared by following the procedure as depicted in Scheme VIII. The reaction comprises reacting a compound of Formula XIIg with a compound of Formula XIIh to give a compound of Formula XIIff.

The reaction of a compound of Formula XIIg with a compound of Formula XIIh can be carried out an organic solvent for example, toluene, hexane or benzene in the presence of a base for example, triethylamine, pyridine, N-methylmorpholine or diisopropylethylamine.

Particular illustrative compounds include the following:
2-(3-Benzyl-3-aza-bicyclo[3.1.0]hex-6-ylamino)-6-(2-chlorophenyl)-8-(1H-tetrazol-5-ylmethyl)-8H-pyrido[2,3-d]pyrimidin-7-one (Compound No. 62)

Particular illustrative compounds which may be produced by Scheme I, include those in the Table below:

| | |
|---|---|
| Compound | |
| No | Structure |

| | |
|---|---|
| 1. | 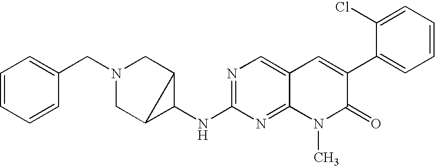 |
| 2. | 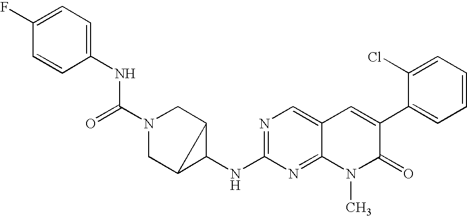 |
| 3. | 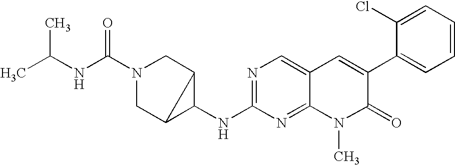 |
| 4. | 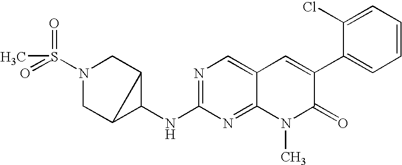 |
| 5. | 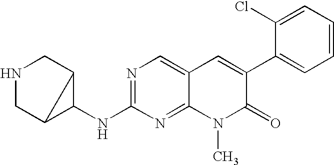 |
| 6. | 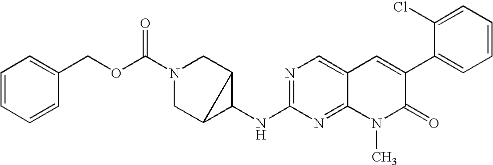 |
| 7. | 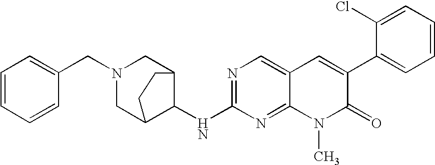 |
| 8. | 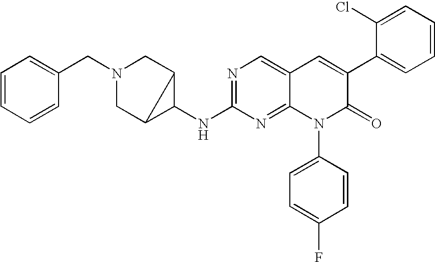 |
| 9. | 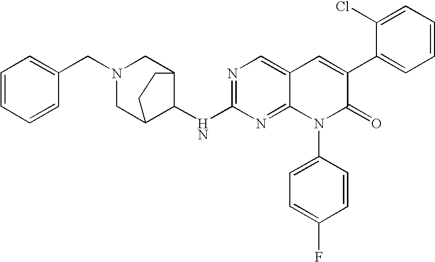 |
| 10. | 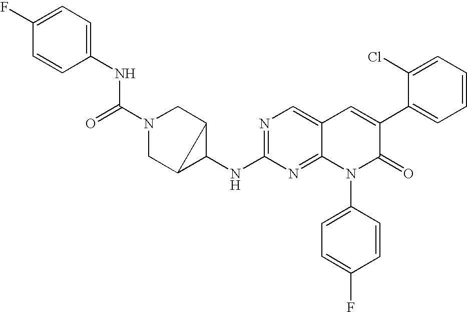 |
| 11. | 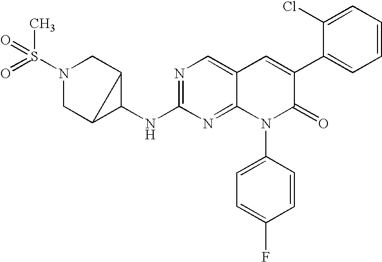 |
| 12. | 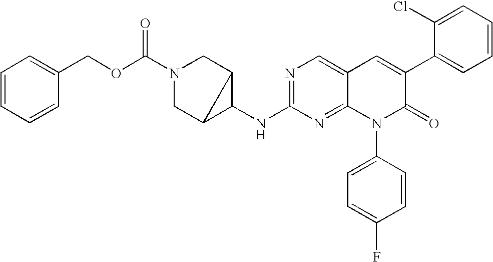 |
| 13. | 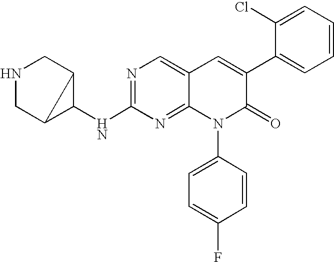 |
| 14. | 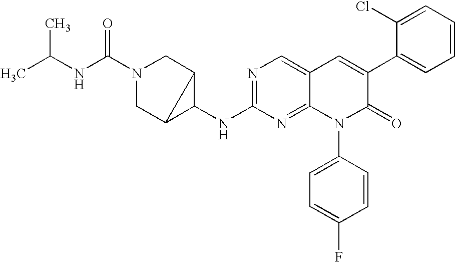 |
| 15. | 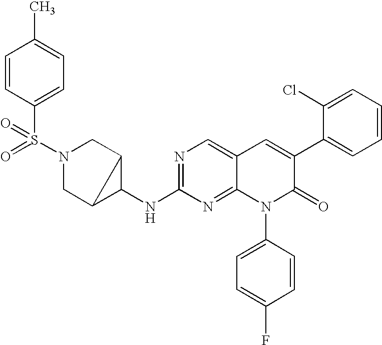 |
| 16. | 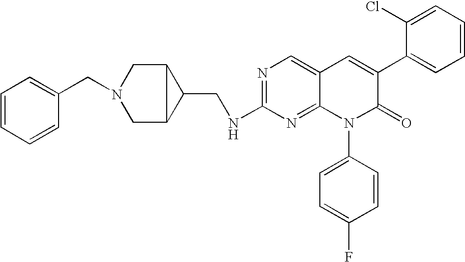 |
| 17. | 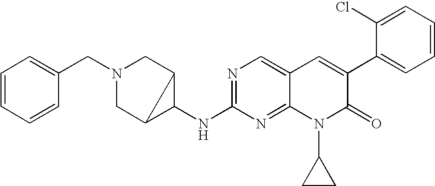 |
| 18. | 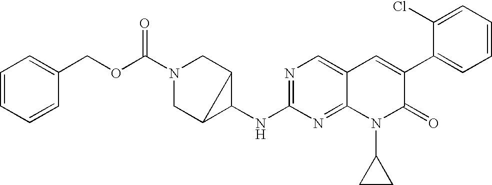 |
| 19. | 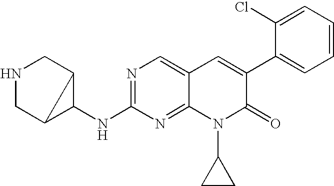 |
| 20. | 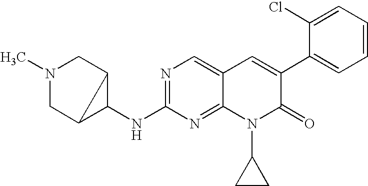 |
| 21. | 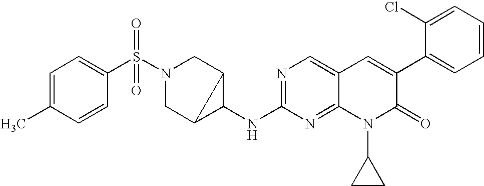 |
| 22. | 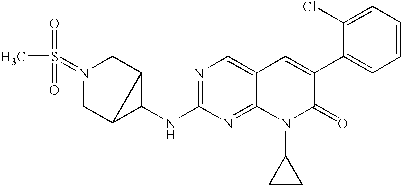 |
| 23. | 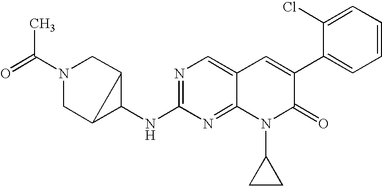 |
| 24. | 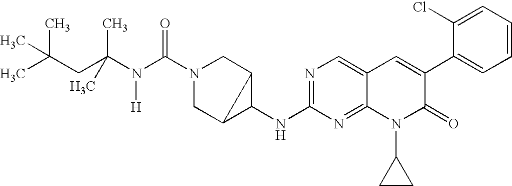 |
| 25. | 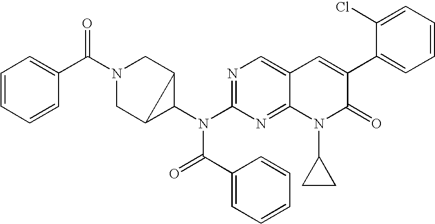 |
| 26. | 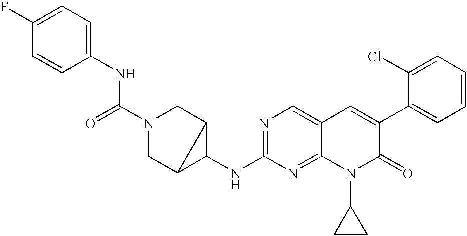 |
| 27. | 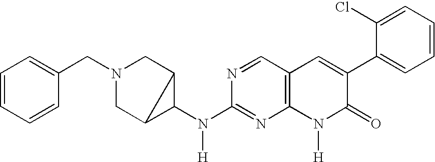 |
| 28. | 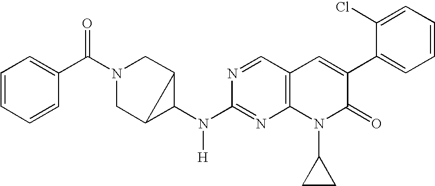 |
| 29 | 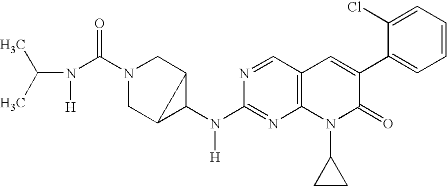 |
| 30 | 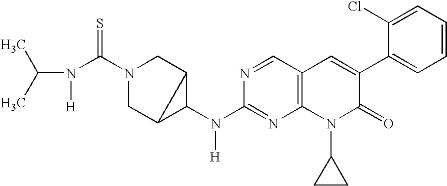 |
| 31 | 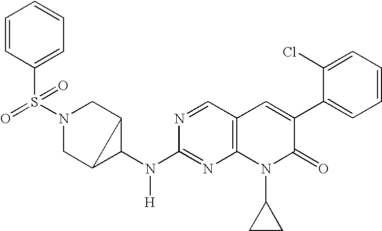 |
| 32 | 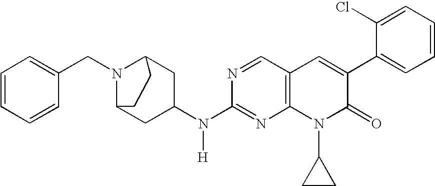 |
| 33 | 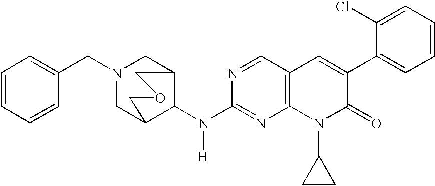 |
| 34 | 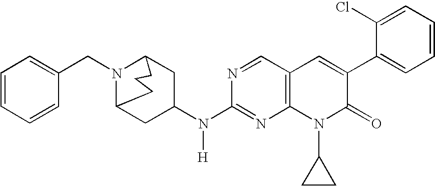 |
| 35 | 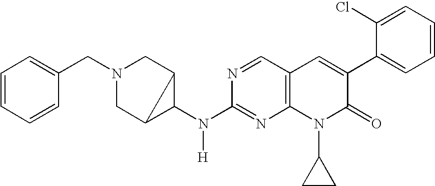 |
| 36 | 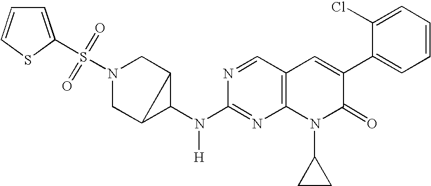 |
| 37 | 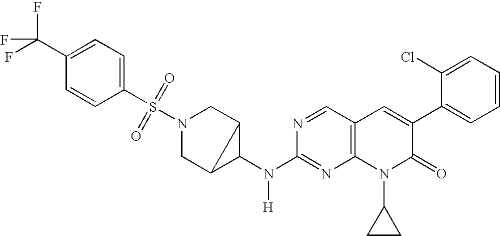 |
| 38 | 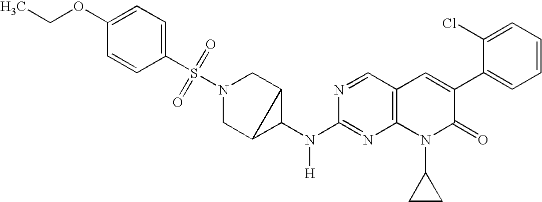 |
| 39 | 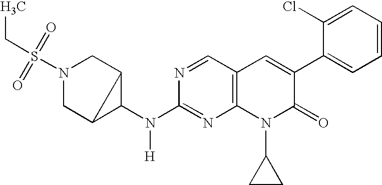 |
| 40 | 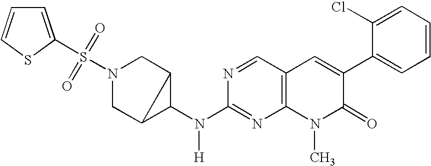 |
| 41 | 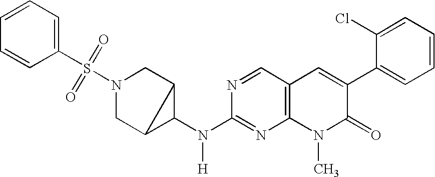 |
| 42 | 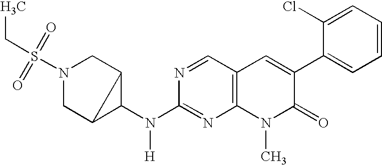 |
| 43 | 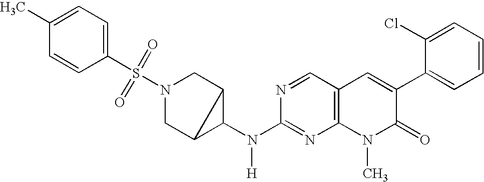 |
| 44 | 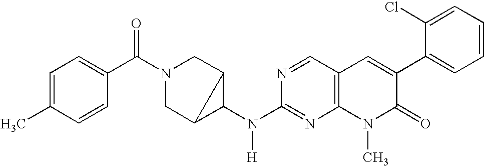 |
| 45 | 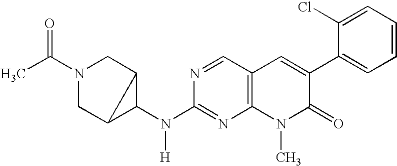 |
| 46 | 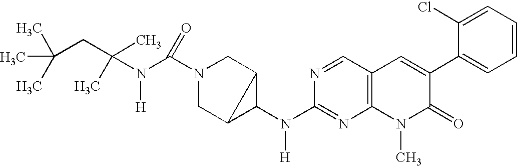 |
| 47 | 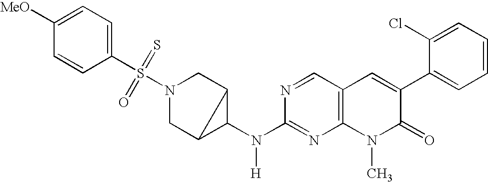 |
| 48 | 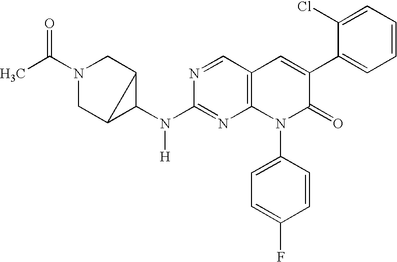 |
| 49 | 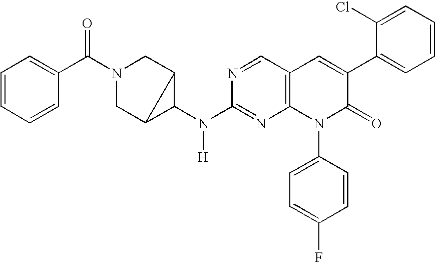 |
| 50 | 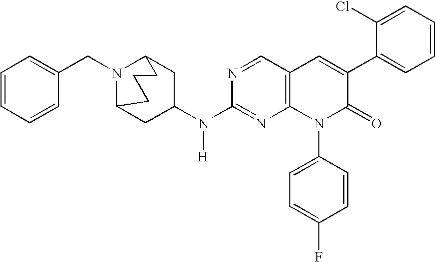 |
| 51 | 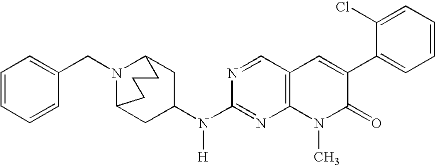 |
| 52 | 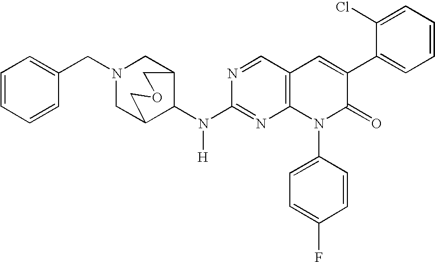 |
| 53 | 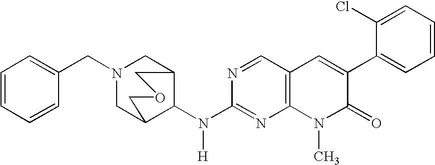 |
| 54 | 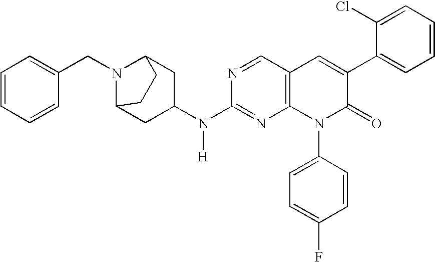 |
| 55 | 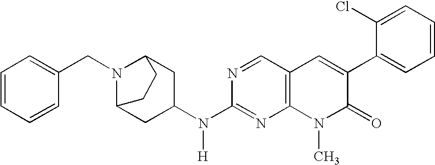 |
| 56 | 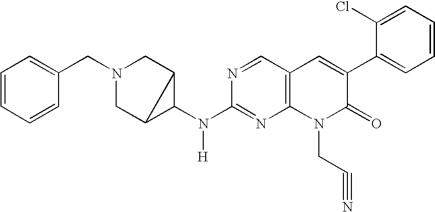 |
| 57 | 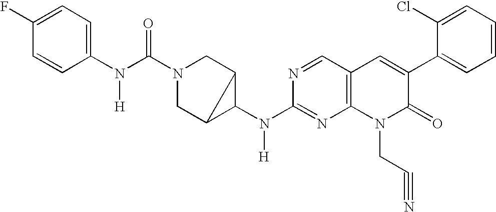 |
| 58 | 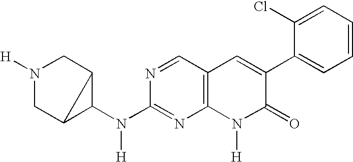 |
| 59 | 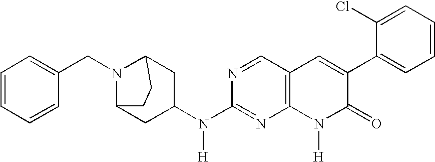 |
| 60 | 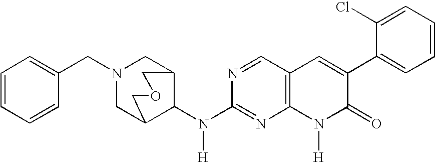 |
| 61 | 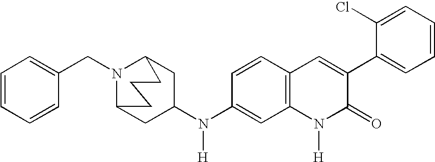 |
| 62 | 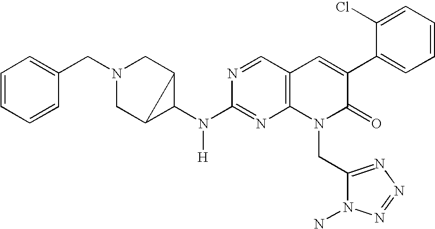 |
| 63 | 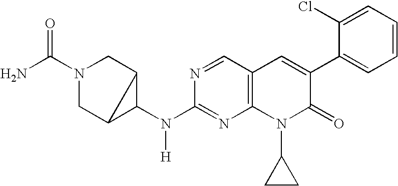 |
| 64 | 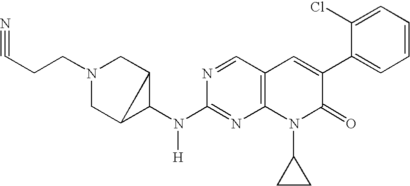 |
| 65 | 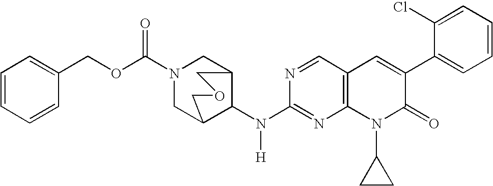 |
| 66 | 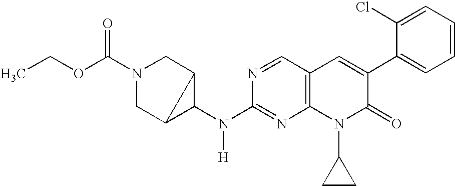 |
| 67 | 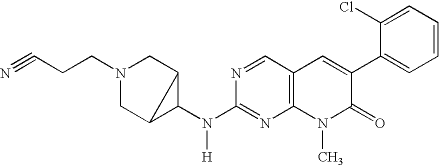 |
| 68 | 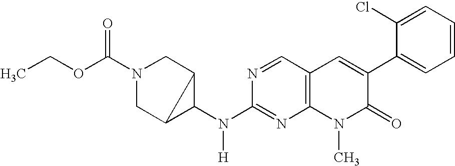 |
| 69 | 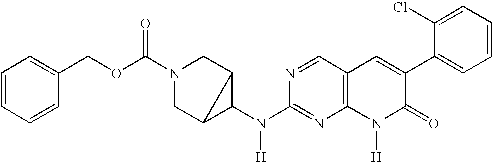 |
| 70 | 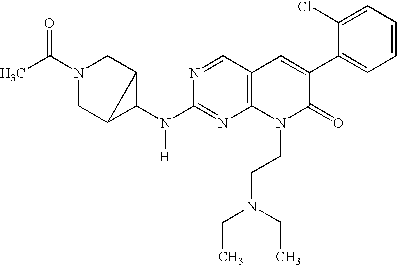 |
| 71 | 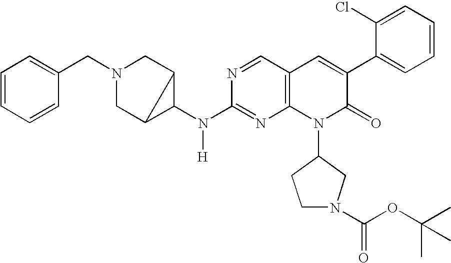 |
| 72 | 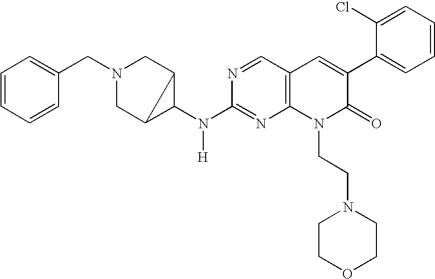 |
| 73 | 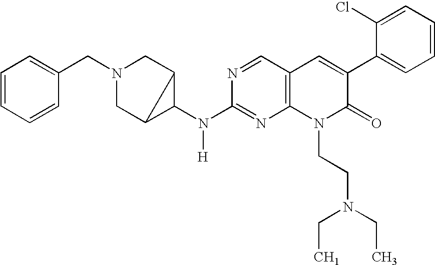 |
| 74 | 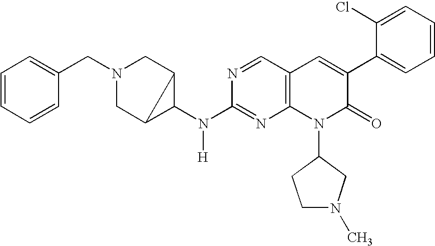 |
| 75 | 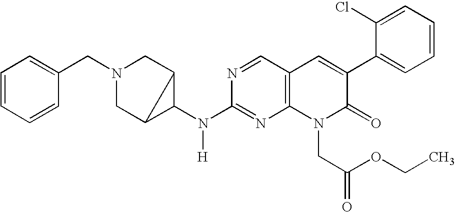 |
| 76 | 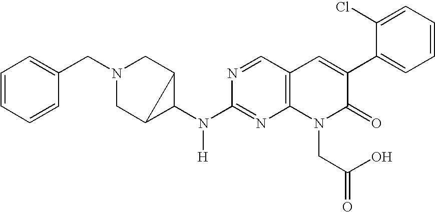 |
| 77 | 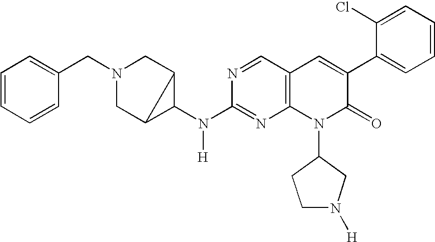 |
| 78 | 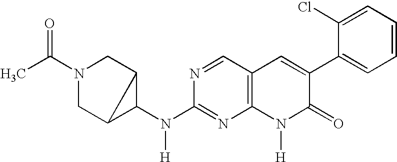 |
| 79 | 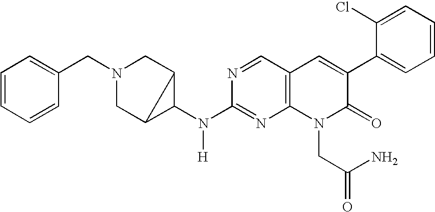 |
| 80 | 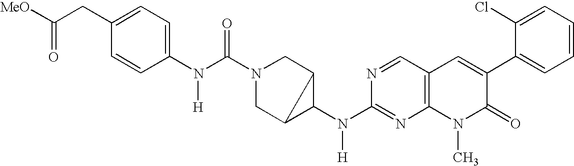 |
| 81 | 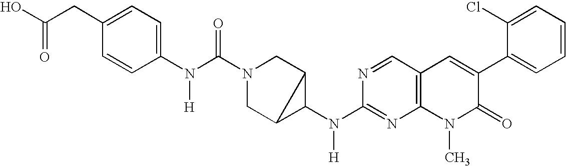 |

The examples set forth demonstrate general synthetic procedures for the preparation of representative compounds. The examples are provided to illustrate particular aspects of the disclosure and do not limit the scope of the present invention.

### EXPERIMENTAL

### Example 1

### Synthesis of 3-benzyl-3-aza-bicyclo[3.1.0]hex-6-ylamine (Formula XXIII)

The title compound was prepared following procedures as described in EP 0413455 or Synlett, (1996), 1097-1099 and 1100-1102.

### Example 2

### Synthesis of 3-benzyl-8-amino-3-aza-bicyclo[3.2.1]octane

### Step a: 3-Benzyl-3-aza-bicyclo[3.2.1]octane-8-one

The title compound was prepared following procedures as described in J. Med. Chem., Vol. 37, (1994), 2831-2840.

### Step b: 3-Benzyl-3-aza-bicyclo[3.2.1]octan-8-one oxime

The title compound was prepared following procedures as described in J. Heterocyclic Chem., Vol. 19, (1982), 485.

### Step c: 3-benzyl-8-amino-3-azabicyclo[3.2.1]octane

To a suspension of lithium aluminum hydride (0.39 g, 10.4 mmol) in tetrahydrofuran (15 ml) at −20° C. was added a solution of the compound obtained from step b above (1.2 g, 5.2 mmol) in tetrahydrofuran drop by drop. The reaction mixture was allowed to warm to room temperature and refluxed for 2-4 hours. Excess of lithium aluminum hydride was decomposed with ethylacetate, aqueous sodium hydroxide and then finally with water. The organic layer was concentrated under reduced pressure and the residue thus obtained was purified by column chromatography using chloroform, methanol and triethylamine (95:5:1) solvent mixture as eluent to furnish the title compound. Yield=0.660 g.

¹H NMR (CDCl₃): δ 3.61 (brs, 2H, —NH₂), 3.52-3.47 (d, 2H, J=13.5 Hz, —CH₂Ar), 3.38-3.36 (t, 1H, J=1.8 & 1.5 Hz, —CH), 2.97 (s, 1H, —CH), 2.79 (s, 1H, —CH), 2.67-2.66 (d, 1H, J=4.2 Hz, —CH), 2.50-2.49 (d, 2H, J=3.6 Hz, —CH₂), 2.43-2.39 (d, 2H, J=11.4 Hz, —CH₂), 2.13-2.10 (d, 2H, J=10.5 Hz, —CH₂) and 1.95 (brs, 1H, —CH); Mass spectrum (m/z, +ve ion mode): 217 (M⁺+1).

Analogues of 3-benzyl-8-amino-3-azabicyclo[3.2.1]octane described below, can be prepared by using appropriate ketone in place of cyclopentanone, respectively, as applicable in each case.
3-Benzyl-3-aza-bicyclo[3.1.1]hept-6-ylamine
3-Benzyl-6-methyl-3-aza-bicyclo[3.2.1]oct-8-ylamine
3-Benzyl-7-methyl-3-aza-bicyclo[3.1.1]hept-6-ylamine
7-Benzyl-3-oxo-7-aza-bicyclo[3.3.1]non-9-ylamine

### Example 3

### Synthesis of 6-amino-3-aza-bicyclo[3.1.0]hexane-3-carboxylic acid benzyl ester

### Step a: 3-Benzyl-3-azabicyclo[3.1.0]hex-6-yl)-carbamic acid tert-butyl ester

To a solution of the compound of Formula XXII (15 g, 75.37 mmol) in dichloromethane (175 ml), was added triethyl amine (21.0 ml, 150.75 mmol) and di-tert-butyl dicarbonate (24.6 g, 113 mmol) at 0° C. and stirred for 4 hours at room temperature. The reaction mixture was slowly poured over a saturated solution of sodium bicarbonate, extracted with ethyl acetate, washed with water and dried over anhydrous sodium sulphate. The organic layer was concentrated under reduced pressure and the residue thus obtained was purified by column chromatography using ethylacetate in hexane (15:85) solvent mixture to furnish the title compound. Yield=14 g.

### Step b: (3-Aza-bicyclo[3.1.0]hex-6-yl)-carbonic acid tert-butyl ester

To a solution of the compound obtained from step a above (10 g, 34.72 mmole) in methanol (150 ml), was added palladium on carbon (10%, 7 g) and the reaction mixture was refluxed for 30 minutes. To it was added ammonium formate (10.94 g, 173.61 mmol) slowly over a period of 15 minutes and stirred for 15 minutes at 70° C. Filtered the reaction mixture through a prewashed celite pad. The organic solvent was evaporated under reduced pressure, added a saturated solution of sodium bicarbonate, extracted with ethyl acetate, washed with water and dried over anhydrous sodium sulphate. The organic layer was concentrated under reduced pressure to furnish the title compound. Yield=4.3 g.

### Step c: 6-Tert-butyloxycarbonylamino-3-aza-bicyclo[3.1.0]hexane-3-carboxylic acid benzyl ester

To a solution of the compound obtained from step b above (2.20 g, 11.11 mmol) in dichloromethane (30 ml), was added triethylamine (3.87 ml, 27.77 mmol) and cooled to 0° C. To the resulting mixture was added benzyl chloroformate (2.4 ml, 16.66 mmol) dropwise into the reaction mixture at 0° C. The reaction mixture was poured into saturated solution of sodium bicarbonate, extracted with ethyl acetate, washed with water, dried over anhydrous sodium sulphate, filtered and evaporated under reduced pressure to furnish the title compound. Yield=4.5 g.

### Step d: 6-Amino-3-aza-bicyclo[3.1.0]hexane-3-carboxylic acid benzyl ester

To a solution of the compound obtained from step c above (11.4 g, 12.46 mmol) in methanol (25 ml), was added methanolic hydrochloric acid (60 ml) and stirred for 16 hours. The reaction mixture was cooled to 0° C. followed by addition of saturated solution of sodium bicarbonate. The organic solvent was evaporated under reduced pressure, extracted with ethyl acetate, washed with water, dried over anhydrous sodium sulphate and filtered. The organic layer was concentrated under reduced pressure to furnish the title compound.

¹H NMR: δ 7.33-7.08 (m, 5H, Ar—H), 5.09 (s, 2H, —CH₂Ph), 4.68 (brm, 1H, —NH), 3.73 (d, 2H, J=12.0 Hz, —NCH₂), 3.53-3.36 (brm, 3H, —NCH₂ & —NHCH), 3.10-3.07 (m, 1H, —CH), 2.46-2.43 (m, 1H, —CH) and 1.44 (s, 9H, —C(CH₃)₃); Mass spectrum (m/z, +ve ion mode): 333 [M⁺+1].

### Example 4

### Synthesis of 6-amino-3-aza-bicyclo[3.1.0]hexane-3-carboxylic acid-(4-fluorophenyl)-amide

### Step a: 3-(4-Fluorophenylcarbamoyl)-3-aza-bicyclo[3.1.0]hex-6-yl]-carbonic acid tert butyl ester

To a solution of the compound (Example 3, step b) (1.5 g, 7.75 mmol) in dichloromethane (20 ml), was added 4-fluorophenyl isocyanate (1.24 g, 9.09 mmol) and stirred for 3 hours. The reaction mixture was poured into water, extracted with dichloromethane and washed with water. The dichloromethane layer was dried over anhydrous sodium sulphate, filtered and evaporated under reduced pressure. The residue thus obtained was purified by column chromatography using methanol in dichloromethane (1:49) solvent mixture as eluent to furnish the title compound. Yield=2.1 g.

### Step b: 6-Amino-3-aza-bicyclo[3.1.0]hexane-3-carboxylic acid-(4-fluorophenyl)-amide

To a solution of the compound (Example 4, step a) (0.3 g, 8.95 mmol) in dichloromethane (5 ml) was added trifluoroacetic acid (0.25 g, 2.24 mmol) and stirred at room temperature for 16 hours. The reaction mixture was poured into a cold (0° C.) saturated solution of sodium bicarbonate and extracted with ethyl acetate. The ethyl acetate layer was washed with water, dried over anhydrous sodium sulphate, filtered and evaporated under vacuum to afford the title compound. Yield: 0.20 g.

¹H NMR (DMSO-d₆): δ 8.32-8.24 (brm, 2H, Ar—H), 7.50-7.45 (brm, 1H, Ar—H), 7.08-7.02 (brm, 1H, Ar—H), 3.74-3.66 (brm, 4H, 2×-NCH₂), 3.44-3.41 (brm, 2H, 2×-CH), 2.40 (s, 1H, —NCH) and 1.99-1.90 (brs, 2H, 2×-CH); Mass spectrum (m/z, +ve ion mode): 236 [M⁺+1]; m.p: 149.9-150.5° C.

### Example 5

### Synthesis of 2-(3-benzyl-3-aza-bicyclo[3.2.1]hex-6-ylamino)-6-(2-chlorophenyl)-8-methyl-8H-pyrido[2,3-d]pyrimidin-7-one (Compound No. 1)

### Step a: 4-Methylamino-2-methylsulfonyl-pyrimidine-5-carboxylic acid ethyl ester

To a suspension of ethyl-4-chloro-2-methylthio-5-pyrimidine-carboxylate (commercially available) (8.0 g, 34 mmol) in dry tetrahydrofuran (60 ml), was added triethylamine (4.3 g, 42 mmol) and aqueous methylamine (40%, 3.2 g, 36.2 mmol) at room temperature and stirred for 2 hours. The organic solvent was evaporated under reduced pressure followed by addition of water. It was then extracted with ethylacetate, washed with water, dried over anhydrous sodium sulphate, filtered and evaporated under reduced pressure. The residue thus obtained was purified by column chromatography using ethylacetate in hexane (1:9) solvent mixture as eluent. Yield=6.2 g.

### Step b: 4-Methylamino-2-methylsulphonyl-pyrimidin-5-yl)-methanol

To a suspension of lithium aluminium hydride (1.21 g, 32 mmol) in dry tetrahydrofuran (60 ml) at −70° C., was added a solution of the compound obtained from step a above (6.0 g, 26 mmol) in tetrahydrofuran (20 ml) dropwise. The reaction mixture was stirred between −70° C.-60° C. for 1 hour and then at room temperature till completion. The reaction mixture was cooled to 0° C. and diluted with ethylacetate, followed by addition of 30% aqueous solution of sodium hydroxide. The reaction mixture was then filtered through a celite pad and washed with ethylacetate. The filtrate was extracted with ethylacetate and the organic layer was washed with water, dried over anhydrous sodium sulphate, filtered and evaporated under reduced pressure. The residue thus obtained was purified by column chromatography using ethylacetate in hexane (1:1) solvent mixture as eluent to furnish the title compound. Yield=4.0 g.

### Step c: 4-Methylamino-2-methylsulphonyl-pyrimidin-5-carboxldehyde

To a suspension of compound obtained from step b above (3.8 g, 20.7 mmol) in dichloromethane (100 ml), was added manganese dioxide (12.7 g, 145 mmol) at room temperature and stirred for 24-36 hours. The reaction mixture was filtered over a celite pad and evaporated under reduced pressure. The residue thus obtained was purified by column chromatography using ethylacetate in hexane (1:4) solvent mixture as eluent to furnish the title compound. Yield=3.2 g.

### Step d: 6-(2-Chlorophenyl)-8-methyl-2-methyl-sulphonyl-8H-pyrido[2,3-d]pyrimidin-7-one

To a solution of the compound obtained from step c above (3.2 g, 17.7 mmol) in N-methylpyrrolidinone (20 ml), was added 2-chlorophenyl acetic acid methyl ester (4.9 g, 26.6 mmol) and potassium carbonate (7.4 g, 53.04 mmol) and heated at 110° C. for 2 hours. The reaction mixture was diluted with ethylacetate and poured into water. It was then extracted with ethyl acetate, washed with water, dried over anhydrous sodium sulphate, filtered and evaporated under reduced pressure. The residue thus obtained was purified by column chromatography using ethylacetate in hexane (1:3) as eluent. Yield=3.2 g.

### Step e: 6-(2-Chlorophenyl)-2-methane sulphonyl-8-methyl-8H-pyrido[2,3-d]pyrimidin-7-one

To a solution of the compound obtained from step d above (1.5 g, 4.7 mmol) in chloroform (20 ml) was added m-chloroperbenzoic acid (70%) (3.5 g, 14.2 mmol) at 0° C. and stirred at room temperature for 30 minutes. To it was added a saturated solution of aqueous sodium bisulphate followed by aqueous sodium bicarbonate solution at 0° C. The reaction mixture was then extracted with dichloromethane and the organic layer was washed with water, dried over anhydrous sodium sulphate, filtered and evaporated under reduced pressure. The residue thus obtained was washed thoroughly with hexane to furnish the title compound. Yield=1.1 g

### Step f: 2-(3-Benzyl-3-azabicyclo[3.1.0]hex-6-ylamino)-6-(2-chlorophenyl)-8-methyl-8H-pyrido[2,3-d]pyrimidin-7-one

To the compound obtained from step e above (0.1 g, 0.286 mmol), was added 3-benzyl-3-aza-bicyclo[3.1.0]hex-6-ylamine (0.1 g, 0.572 mmol) and heated to 120° C. for 2 hours. The reaction mixture was diluted with dichloromethane and the compound was purified by column chromatography using methanol in dichloromethane (1:9) solvent mixture as eluent to furnish the title compound. Yield=60 mg.

¹H NMR (CDCl₃): δ 8.49 (s, 1H, Ar—H), 7.61 (s, 1H, Ar—H), 7.49 (s, 2H, Ar—H), 7.33 (brm, 7H, Ar—H), 3.76 (brs, 3H, —NCH₃), 3.35 (brs, 2H, —CH₂Ar), 3.18 (brs, 3H, —NCH₂ & —NCH), 2.67-2.64 (brm, 1H, —NCH) and 1.63 (brs, 3H, 2×-CH & —NCH); Mass spectrum (m/z, +ve ion mode): 460 [M⁺+1+2], 458 [M⁺+1]; m.p: 92-94° C.

The analogues of 2-(3-benzyl-3-aza-bicyclo[3.1.0]hex-6-ylamino)-6-(2-chlorophenyl)-8-methyl-8H-pyrido[2,3-d]pyrimidin-7-one (Compound No. 1) described below can be prepared by using appropriate amine in place of methylamine or 3-benzyl-3-aza-bicyclo[3.1.0]hex-6-ylamine, respectively as applicable in each case.

### 2-(3-Benzyl-3-aza-bicyclo[3.2.1]oct-8-ylamino)-6-(2-chlorophenyl)-8-methyl-8H-pyrido[2,3-d]pyrimidin-7-one (Compound No. 7)

¹H NMR (CDCl₃): δ 8.47 (bs, 1H, Ar—H), 7.27-7.56 (m, 10H, 10Ar—H & —NH), 3.72 (s, 3H, —NCH₃), 3.55 (s, 2H, —CH₂Ar), 3.38 (brs, 1H, —NCH), 2.58-2.61 (d, 2H, J=9.0 Hz, —NCH₂), 2.48 (d, 2H, J=12.0 Hz, —NCH₂), 2.37 (brs, 2H, 2×-CH) and 1.93-1.86 (m, 4H, 2×-CH₂); Mass spectrum (m/z, +ve ion mode): 488 [M⁺+1+2], 486 [M⁺+1]; m.p: 88-92° C.

### 2-(3-Benzyl-3-aza-bicyclo[3.1.0]hex-6-ylamino)-6-(2-chlorophenyl)-8-(4-fluorophenyl)-8H-pyrido[2,3-d]pyrimidin-7-one (Compound No. 8)

¹H NMR (DMSO-d₆): δ 8.65 (s, 1H, Ar—H), 7.88-7.92 (brm, 1H, Ar—H), 3.36 (m, 2H, —NCH₂), 3.52-3.50 (2H, J=5.7 Hz, —CH₂Ar & —NCH₂), 2.59-2.50 (m, 1H, —NCH) and 2.21-2.32-2.35 (brm, 2H, 2×-CH); Mass spectrum (m/z, +ve ion mode): 540 [M⁺+1+2], 538 [M⁺+1]; m.p: 236.8-238.9° C.

### 2-(3-Benzyl-3-aza-bicyclo[3.2.1]oct-8-ylamino)-6-(2-chlorophenyl)-8-(4-fluorophenyl)-8H-pyrido[2,3-d]pyrimidin-7-one (Compound No. 9)

¹H NMR (DMSO-d₆): δ 8.67 (s, 1H, Ar—H), 7.90-7.88 (brm, 1H, Ar—H), 7.73-7.25 (m, 13H, 12Ar—H & —NH), 4.12-4.07 (dd, 2H, J=6.0 Hz each, —CH₂Ar), 3.61-3.46 (m, 4H, 2×-NCH₂), 3.28-3.16 (m, 5H, 2×-CH₂ & —CH) and 2.07-2.01 (m, 2H, —CH₂); Mass spectrum (m/z, +ve ion mode): 568 [M⁺+1+2], 566 [M⁺+1]; m.p: 97-101.8° C.

### 2-[(3-Benzyl-3-aza-bicyclo[3.1.0]hex-6-ylmethyl)-amino]-6-(2-chlorophenyl)-8-(4-fluorophenyl)-8H-pyrido[2,3-d]pyrimidin-7-one (Compound No. 16)

¹H NMR (CDCl₃): δ 7.60 (s, 1H, Ar—H), 7.45 (s, 1H, Ar—H), 7.44-7.41 (m, 2H, Ar—H), 7.39-7.15 (m, 1H, Ar—H), 3.64-3.61 (m, 2H, —CH₂Ar), 2.93 (brm, 4H, 2×-NCH₂), 2.31 (brs, 2H, —NHCH₂), 2.01 (brs, 1H, —CHCH₂NH) and 1.77 (brs, 2H, 2×-CH); Mass spectrum (m/z, +ve ion mode): 554 [M⁺+1+2], 552 [M⁺+1]; m.p: 104.9-109.6° C.

### 2-(3-Benzyl-3-aza-bicyclo[3.1.0]hex-6-ylamino)-6-(2-chlorophenyl)-8-cyclopropyl-8H-pyrido[2,3-d]pydrimidin-7-one (Compound No. 17)

¹H NMR (CDCl₃): δ 8.40 (s, 1H, Ar—H), 7.48-7.44 (m, 2H, Ar—H), 7.36-7.23 (m, 8H, Ar—H), 5.59 (brs, 1H, —NH), 3.63 (s, 2H, —CH₂Ar), 3.27 (brs, 1H, —NHCH), 3.16 (d, 2H, J=9.0 Hz, —NCH₂), 2.96 (brm, 1H, —NCH), 2.50 (d, 2H, J=6.0 Hz, —NCH₂), 1.62 (brs, 2H, —CH₂) and 1.37-1.28 (brm, 2H, —CH₂); Mass spectrum (m/z, +ve ion mode): 486 [M⁺+1+2], 484 [M⁺+1]; m.p: 176.5-179.0° C.

### 2-(3-Benzyl-3-aza-bicyclo[3.1.0]hex-6-ylamino)-6-(2-chlorophenyl)-8H-pyrido[2,3-d]pyrimidin-7-one (Compound No. 27)

¹H NMR (DMSO-d₆): δ 7.74 (s, 1H, Ar—H), 7.51 (s, 1H, Ar—H), 7.39-7.30 (m, 9H, Ar—H), 3.63-3.48 (brm, 4H, —CHAr & —NCH₂), 3.07 (brm, 3H, —NCH₂ & —NCH), 2.43-2.40 (m, NCH₂) and 1.70-1.60 (m, 2H, 2×-CH); Mass spectrum (m/z+ve ion mode): 446 [M⁺+1+2], 444 [M⁺+1]; m.p: 250° C.

### 2-(1-Benzlpiperidin-4-ylamino)-6-(2-chlorophenyl)-8-cyclopropyl-8H-pyrido[2,3-d]pyrimidin-7-one (Compound No. 32)

¹H NMR (CDCl₃): δ 8.44 (s, 1H, Ar—H), 7.51-7.25 (brm, 1H, 10 Ar—H & —NH), 4.34 (brm, 1H, —NCH), 4.08-4.04 (brm, 2H, —CH₂Ar), 3.92-3.84 (brm, 3H, 3×-NCH), 3.59-3.56 (brm, 2H, —CH₂), 2.97-2.81 (brm, 4H, 2×-CH₂), 2.16 (brs, 2H, —CH₂), 1.26 (brm, 2H, —CH₂) and 0.96-0.86 (brm, 2H, —CH₂); Mass spectrum (m/z, +ve ion mode): 514 [M⁺+1+2], 512 [M⁺+1]; m.p: 246.4-262° C.

### 2-(7-Benzyl-3-oxa-7-aza-bicyclo[3.3.1]non-9-ylamino)-6-(2-chlorophenyl)-8-cyclopropyl-8H-pyrido[2,3-d]pyrimidin-7-one (Compound No. 33)

¹H NMR (CDCl₃): δ 7.49 (s, 1H, Ar—H), 7.48-7.30 (brm, 10H, Ar—H), 4.45-4.42 (brm, 1H, —NCH), 3.60 (s, 2H, —CH₂Ar), 3.39-3.20 (brm, 4H, 2×-OCH₂), 2.95 (brm, 1H, —NCH), 1.99-1.95 (brm, 2H, —NCH₂), 1.82-1.80 (brm, 2H, —NCH), 1.72 (brm, 2H, 2×-CH), 1.69-1.67 (brm, 2H, —CH₂) and 1.31-1.26 (brm, 2H, —CH₂); Mass spectrum (m/z, +ve ion mode): 530 [M⁺+1+2], 528 [M⁺+1]; m.p: 139-150.4° C.

### 2-(9-Benzyl-9-aza-bicyclo[3.3.1]non-3-ylamino)-6-(2-chlorophenyl)-8-cyclopropyl-8H-pyrido[2,3-d]pyrimidin-7-one (Compound No. 34)

¹H NMR (CDCl₃): δ 8.60 (s, 1H, Ar—H), 7.58 (s, 1H, Ar—H), 7.49 (s, 1H, Ar—H), 7.35-7.33 (m, 3H, Ar—H), 7.11 (brm, H, Ar—H), 6.93 (brs, 2H, Ar—H), 3.99-3.94 (d, 1H, J=15.0 Hz, —CHPh), 3.71-3.67 (d, 1H, J=12.0 Hz, —CHAr), 3.58 (brs, 1H, —NH), 3.15-3.13 (t, 1H, J=6.00 Hz, —NCH), 3.00-2.97 (brm, 2H, —CH₂), 2.83-2.81 (m, 1H, —CH), 2.69-2.67 (m, 1H, —CH), 1.99-1.57 (brm, 4H, 2×-CH₂), 1.27 (brm, 4H, 2×-CH₂) and 0.90-0.78 (brm, 2H, —CH₂); Mass spectrum (m/z, +ve ion mode): 528 [M⁺+1+2], 526 [M⁺+1]; m.p: 183.7-206.9° C.

### 2-(3-Benzyl-3-aza-bicyclo[3.1.0]hex-6-ylamino)-6-(2-chlorophenyl)-8-cyclopropyl-8H-pyrido[2,3-d]pyrimidin-7-one (Compound No. 35)

¹H NMR (CDCl₃): δ 8.39 (s, 1H, Ar—H), 7.46-7.43 (brm, 2H, Ar—H), 7.36-7.24 (brm, 8H, Ar—H), 3.65 (brm, 2H, —CH₂Ph), 3.41-3.37 (m, 2H, —NCH₂), 3.08-3.05 (m, 2H, —NCH₂), 2.93 (brm, 1H, —NCH), 2.47-2.44 (brm, 2H, —NCH & —CH), 1.42 (brm, 1H, —CH), 1.31-1.26 (brm, 2H, —CH₂) and 0.94 (brm, 2H, —CH₂). Mass spectrum (m/z, +ve ion mode): 486 [M⁺+1+2], 484 [M⁺+1].

### 2-(9-Benzyl-9-aza-bicyclo[3.3.1]non-3-ylamino)-6-(2-chlorophenyl)-8-(4-fluorophenyl-8H-pyrido[2,3-d]pyrimidin-7-one (Compound No. 50)

¹H NMR (CDCl₃): δ 8.68 (s, 1H, Ar—H), 7.57 (s, 1H, Ar—H), 7.56-7.12 (m, 11H, Ar—H), 6.75 (s, 2H, Ar—H), 4.20-3.86 (m, 2H, —CH₂Ar), 3.46-3.41 (m, 2H, −2×-NCH), 2.97-2.93 (m, 1H, —NHCH), 2.71-2.52 (m, 4H, 2×-CH₂), 2.2-2.1 (m, 2H, —CH₂), 2.02-1.94 (m, 2H, —CH₂) and 1.26 (s, 2H, —CH₂); Mass spectrum (m/z, +ve ion mode): 582 [M⁺+1+2], 580 [M⁺+1]; m.p: 195-200° C.

### 2-(9-Benzyl-9-aza-bicyclo[3.3.1]non-3-ylamino)-6-(2-chlorophenyl)-8-methyl-8H-pyrido[2,3-d]pyrimidin-7-one (Compound No. 51)

Mass spectrum (m/z, +ve ion mode): 502 [M⁺+1+2], 500 [M⁺+1]; m.p: 76.9-82.0° C.

### 2-(7-Benzyl-3-oxa-7-aza-bicyclo[3.3.1]non-9-ylamino)-6-(2-chlorophenyl)-8-(4-fluorophenyl)-8H-pyrido[2,3-d]pyrimidin-7-one (Compound No. 52)

Mass spectrum (m/z, +ve ion mode): 584 [M⁺+1+2], 582 [M⁺+1]; m.p: 107.4-110.4° C.

### 2-(7-Benzyl-3-oxa-7-aza-bicyclo[3.3.1]non-9-ylamino)-6-(2-chlorophenyl)-8-methyl-8H-pyrido[2,3-d]pyrimidin-7-one (Compound No. 53)

¹H NMR (CDCl₃): δ 8.45 (s, 1H, Ar—H), 7.62-7.44 (m, 4H, Ar—H), 7.36-7.24 (m, 6H, Ar—H), 4.23-4.18 (m, 2H, —OCH₂), 3.91-3.83 (m, 2H, —OCH₂), 3.75 (s, 5H, —NCH₃ & —CH₂Ar), 3.69-3.63 (m, 2H, —NCH₂), 3.48-3.45 (m, 2H, —NCH₂), 2.80-2.76 (m, 1H, —NHCH), 1.68-1.38 (m, 2H, 2×-CH); Mass spectrum (m/z, +ve ion mode): 504 [M⁺+1+2], 502 [M⁺+1]; m.p: 290.2-300.2° C.

### 2-(8-Benzyl-8-aza-bicyclo[3.2.1]oct-3-ylamino)-6-(2-chlorophenyl)-8-(4-fluorophenyl)-8H-pyrido[2,3-d]pyrimidin-7-one (Compound No. 54)

¹H NMR (CDCl₃): δ 8.48 (s, 1H, Ar—H), 8.07 (s, 1H, Ar—H), 7.97-7.95 (m, 1H, Ar—H), 7.64-7.42 (m, 2H, Ar—H), 7.39-7.14 (m, 10H, Ar—H), 3.86-3.74 (m, 3H, 2×-NCH & —NHCH), 3.64 (s, 2H, —CH₂Ar), 3.49-3.39 (m, 2H, —CH₂), 2.59-2.57 (m, 2H, —CH₂) and 1.71-1.64 (m, 4H, −2×-CH₃).

Mass spectrum (m/z, +ve ion mode): 568 [M⁺+1+2], 566 [M⁺+1]; m.p: 139.7-143.4° C.

### 2-(8-Benzyl-8-aza-bicyclo[3.2.1]oct-3-ylamino)-6-(2-chlorophenyl)-8-methyl-8H-pyrido[2,3-d]pyrimidin-7-one (Compound No. 55)

Mass spectrum (m/z, +ve ion mode): 488 [M⁺+1+2], 486 [M⁺+1]; m.p: 147.4-153.8° C.

### [2-(3-Benzyl-3-aza-bicycyclo[3.1.0]hex-6-ylamino)-6-(2-chlorophenyl)-7-oxo-7H-pyrido[2,3-d]pyrimidin-8-yl]-acetonitrile (Compound No. 56)

¹H NMR (CDCl₃): δ 8.48 (brs, 1H, Ar—H), 7.61 (s, 1H, Ar—H), 7.49-7.26 (m, 9H, Ar—H), 5.87 (bs, 1H, —NH), 5.37 (brs, 2H, —CH₂CN), 3.41-3.36 (m, 2H, —CH₂Ar), 2.6 (m, 1H), 2.40-2.35 (m, 2H, —NCH₂), 2.07-1.97 (m, 2H, —NCH₂) and 1.25 (brs, 2H); Mass spectrum (m/z, +ve ion mode): 485 [M⁺+1+2], 483 [M⁺+1]; IR (DCM): 3425, 2986, 1670, 1596, 1537, 1408, 1218.

### 2-(8-Benzyl-8-aza-bicyclo[3.2.1]oct-3-ylamino)-6-(2-chloro-phenyl)-8H-pyrido[2,3-d]pyrimidin-7-one (Compound No. 59)

¹H NMR (CDCl₃): δ 8.47 (brs, 1H, Ar—H), 7.56-7.29 (m, 10H, Ar—H), 3.64-3.48 (m, 4H, —CH₂Ar & 2×—NH), 3.11-3.08 (m, 1H, —NHCH), 2.19-2.00 (m, 2H, —CH₂), 1.66-1.63 (m, 2H, —CH₂), 1.42-1.37 (m, 2H, —CH₂) and 1.25 (s, 2H, —CH₂); Mass spectrum (m/z, +ve ion mode): 474 [M⁺+1+2], 472 [M⁺+1].

### 2-{7-Benzyl-3-oxa-7-aza-bicyclo[3.3.1]non-9-ylamino}-6-(2-chlorophenyl)-8H-pyrido[2,3-d]pyrimidin-7-one (Compound No. 60)

Mass (m/, +ve ion mode): 490 [M⁺+1+2], 488 [M⁺+1].

### 2-{7-Benzyl-3-oxa-7-aza-bicyclo[3.3.1]non-9-ylamino}-6-(2-chlorophenyl)-8H-pyrido[2,3-d]pyrimidin-7-one (Compound No. 61)

¹H NMR (CDCl₃): δ 8.64 (s, 1H, Ar—H), 7.66 (s, 1H, Ar—H), 7.50-7.47 (m, 1H, Ar—H), 7.39-7.32 (m, 3H, Ar—H), 7.18-7.16 (m, 3H, Ar—H), 7.06-7.04 (m, 2H, Ar—H), 3.98-3.93 (d, 1H, J=15.0 Hz, —CHAr), 3.82-3.77 (d, 1H, J=15.0 Hz, —CHAr,), 3.54 (s, 1H, —NCH), 3.13-3.09 (m, 1H, —NCH), 2.82-2.72 (m, 3H, —NHCH & —CH₂), 2.12 (brs, 2H, —CH₂), 1.80-1.62 (m, 4H, −2×-CH₂) and 1.31-1.25 (m, 2H—CH₂); Mass spectrum (m/z, +ve ion mode): 488 [M⁺+1+2], 486 [M⁺+1]; m.p: 240.3-242.1° C.

### 9-[6-(2-chlorophenyl)-8-cyclopropyl-7-oxo-7,8-dihydro-pyrido[2,3-d]pyrimidin-2-ylamino]-3-oxa-7-aza-bicyclo[3.3.1]nonane-7-carboxylic acid benzyl ester (Compound No. 65)

Mass spectrum (m/z, +ve ion mode): 574 [M⁺+1+2], 572 [M⁺+1].

### 6-[6-(2-Chlorophenyl)-8-cyclopropyl-7-oxo-7,8-dihydro-pyrido[2,3-d]pyrimidin-2-ylamino]-3-aza-bicyclo[3.1.0]hexane-3-carboxylic acid ethyl ester (Compound No. 66)

¹H NMR (TFA-d): δ 8.76 (s, 1H, Ar—H), 7.81 (s, 1H, Ar—H), 7.56-7.25 (brm, 4H, Ar—H), 4.35-4.00 (brm, 3H, —OCH₂), 3.81-3.77 (brm, 3H, —NCH₂ & —NCH), 2.91-2.90 (brm, 2H, —NCH₂), 2.21-2.13 (brm, 2H, 2×-CH) 1.50-1.07 (brm, 4H, −2×CH₂) and 0.054-0.051 (t, 3H, J=9.00 Hz, —CH₃); Mass (+ve ion mode) m/z: 468 [M⁺+1+2], 466 [M⁺+1].

### 6-[6-(2-Chlorophenyl)-8-methyl-7-oxo-7,8-dihydro-pyrido[2,-d]pyrimidin-2-ylamino]-3-aza-bicyclo[3.1.0]hexane-3-carboxylic acid ethyl ester (Compound No. 68)

Mass (+ve ion mode) m/z: 442 [M⁺+1+2], 440 [M⁺+1]; m.p: 118.7-125.2° C.

### 6-[6-(2-Chloro-phenyl)-7-oxo-7,8-dihydro-pyrido[2,3-d]pyrimidin-2-ylamino]-3-aza-bicyclo[3.1.0]hexane-3-carboxylic acid benzyl ester (Compound No. 69)

¹H NMR (DMSO-d₆): δ 8.63 (brs, 1H, Ar—H), 7.77 (s, 1H, Ar—H), 7.55-7.52 (m, 1H, Ar—H), 7.45-7.32 (m, 8H, Ar—H), 5.08 (s, 2H, —OCH₂Ar), 3.76-3.69 (m, 2H, —NCH₂), 3.53-3.49 (m, 1H, —CH), 2.56-2.48 (s, 2H, —NCH₂) and 1.82 (s, 2H, 2×-CH); Mass spectrum (m/z, +ve ion mode): 490 [M⁺+1+2], 488 [M⁺+1]; m.p: 257.7-264.4° C.

### Example 6

### Synthesis of 6-[6-(2-chlorophenyl)-8-methyl-7-oxo-7,8-dihydro-pyrido[2,3-d]pyrimidin-2-ylamino]-3-aza-bicyclo[3.1.0]hexane-3-carboxylic acid benzyl ester (Compound No. 6)

To a solution of the compound 6-(2-chlorophenyl)-2-methanesulphonyl-8-methyl-8H-pyrido[2,3-d]pyrimidin-7-one (Example 5, Step d) (0.7 g, 2 mmol) in minimum amount of pyridine (1-5 ml), was added 6-amino-3-bicyclo[3.1.0]hexane-3-carboxylic acid benzyl ester (1.0 g, 42.9 mmol). The resulting reaction mixture was heated for 1 hour at 80-120° C. The organic solvent was removed under reduced pressure and diluted with water.

The compound was extracted with ethylacetate, washed with water, dried over anhydrous sodium sulphate, filtered and evaporated under reduced pressure. The residue thus obtained was purified by column chromatography using ethylacetate in dichloromethane (1:1) solvent mixture as eluent to furnish the title compound. Yield=0.820 g.

¹H NMR (CDCl₃): δ 8.46 (s, 1H, Ar—H), 7.55-7.32 (m, 1H, 10Ar—H & —NH), 5.14 (s, 2H, —CH₂Ar), 3.89-3.38 (m, 8H, —NCH₃, 2×-NCH₂, —NCH) and 1.83 (brs, 2H, 2×-CH];

Mass spectrum (m/z, +ve ion mode): 504 [M⁺+1+2], 502 [M⁺+1]; m.p: 188-189° C.

Analogues of 6-[6-(2-chlorophenyl)-8-methyl-7-oxo-7,8-dihydro-pyrido[2,3-d]pyrimidin-2-ylamino]-3-aza-bicyclo[3.1.0]hexane-3-carboxylic acid benzyl ester (Compound No. 6) described below, can be prepared by using appropriate sulphone in place of 6-(2-chlorophenyl)-2-methanesulphonyl-8-methyl-8H-pyrido[2,3-d]pyrimidin-7-one, respectively, as applicable in each case.

### 6-[6-(2-Chlorophenyl)-8-(4-fluorophenyl)-7-oxo-7,8-dihydro-pyrido[2,3-d]pyrimidin-2-ylamino]-3-aza-bicyclo[3.1.0]hexane-3-carboxylic acid benzyl ester (Compound No. 12)

¹H NMR (CDCl₃): δ 8.51 (s, 1H, Ar—H), 7.67 (s, 1H, Ar—H), 7.45-7.18 (m, 12H, 11Ar—H & —NH), 5.18-5.11 (m, 2H, —CH₂Ar), 3.44-3.10 (m, 5H, 2×—NH₂& —NCH) and 1.63-1.42 (m, 2H, 2×-CH); Mass spectrum (m/z, +ve ion mode): 584 [M⁺+1+2], 582 [M⁺+1]; m.p: 119.3-129.3° C.

### 6-[6-(2-Chlorophenyl)-8-cyclopropyl-7-oxo-7,8-dihydro-pyrido[2,3-d]pyrimidin-2-ylamino]-3-aza-bicyclo[3.1.0]hexane-3-carboxylic acid benzyl ester (Compound No. 18)

¹H NMR (CDCl₃): δ 8.43 (s, 1H, Ar—H), 7.50-7.29 (m, 11H, s, 10Ar—H & —NH), 5.59 (s, 1H, —NH), 5.14 (s, 2H, —OCH₂Ar), 3.90-3.86 (m, 2H, —NCH₂), 3.60-3.55 (brm, 3H, —NCH₂ & —NCH), 2.65 (brm, 1H, —NCH), 2.64 (s, 1H, —CH), 1.84 (s, 1H, —CH) and 1.84-1.64 (m, 4H, 2×-CH₂); Mass spectrum (m/z, +ve ion mode): 530 [M⁺+1+2], 528 [M⁺+1]; m.p: 156.5-161.3° C.

### Example 7

### Synthesis of 6-[6-(2-chlorophenyl)-8-(4-fluorophenyl)-7-oxo-7,8-dihydro-pyrido[2,3-d]pyrimidin-2-ylamino]-3-aza-bicyclo[3.1.0]hexane-3-carboxylic acid-(4-fluorophenyl)-amide (Compound No. 10)

### Step a: 6-(2-Chlorophenyl)-8-(4-fluorophenyl)-2-methanesulphonyl-8H-pyrido[2,3-d]pyrimidin-7-one

The title compound was prepared following the procedure as described for the synthesis of 6-(2-chlorophenyl)-2-methanesulphonyl-8-methyl-8H-pyrido[2,3-d]pyrimidin-7-one (Example 5, Step c) by using 4-fluorophenylamine in place of methylamine.

### Step b: 6-[6-(2-Chlorophenyl)-8-(4-fluorophenyl)-7-oxo-7,8-dihydro-pyrido[2,3-d]pyrimidin-2-ylamino]-3-aza-bicyclo[3.1.0]hexane-3-carboxylic acid (4-fluorophenyl)-amide (Compound No. 10)

To a solution of the compound obtained from step a above (0.1 g, 2.32 mmol) in pyridine (1 ml), was added 4-amino-3,5-dimethyl-piperidine-1-carboxylic acid-(4-fluorophenyl)-amide (0.273 g, 11.6 mmol) and heated at 80° C. for 1 hour. Pyridine was azeotropically removed with toluene under reduced pressure. The residue thus obtained was purified by column chromatography using methanol in chloroform (7:93) solvent mixture as eluent to furnish the title compound. Yield=0.060 g

¹H NMR (DMSO-d₆): δ 8.69 (s, 1H, Ar—H), 8.09 (s, 2H, Ar—H), 7.96-7.91 (d, 2H, J=15.0 Hz, Ar—H), 7.54-7.10 (m, 10H, 9Ar—H & —NH), 7.10-7.04 (m, 2H, Ar—H), 3.24-3.16 (m, 4H, 2×-NCH₂), 1.99 (s, 1H, —NCH) and 1.78 (brs, 2H, 2×-CH); Mass spectrum (m/z, +ve ion mode): 587 [M⁺+1+2], 585 [M+1, 100%]; m.p.: 300.4-302.4° C.

Analogues of 6-[6-(2-chlorophenyl)-8-(4-fluorophenyl)-7-oxo-7,8-dihydro-pyrido[2,3-d]pyrimidin-2-ylamino]-3-aza-bicyclo[3.1.0]hexane-3-carboxylic acid (4-fluorophenyl)-amide described below, can be prepared by using appropriate urea and sulphone in place of 4-amino-3,5-dimethyl-piperidine-1-carboxylic acid-(4-fluorophenyl)-amide and 6-(2-chlorophenyl)-8-(4-fluorophenyl)-2-methanesulphonyl-8H-pyrido[2,3-d]pyrimidine-7-one, respectively, as applicable in each case.

### 6-[6-(2-Chlorophenyl)-8-(4-fluorophenyl)-7-oxo-7,8-dihydro-pyrido[2,3-d]pyrimidin-2-ylamino]-3-aza-bicyclo[3.1.0]hexane-3-carboxylic acid isopropylamide (Compound No. 14)

¹H NMR (CDCl₃): δ 8.52 (s, 1H, Ar—H), 7.69 (s, 1H, Ar—H), 7.49-7.27 (m, 9H, 8Ar—H & —NH), 5.69 (brs, 1H, —NH), 5.13 (brs, 1H, —NH), 3.91-4.0 (m, 3H, —NCH₂), 3.65 (s, 1H, —NCH), 3.22 (brs, 2H, —NCH₂), 3.08 (s, 1H, —NCH), 2.18-2.01 (m, 2H, 2×-CH) and 1.00-0.86 (m, 6H, 2×-CH₃); Mass spectrum (m/z, +ve ion mode): 535 [M⁺+1+2], 533 [M+1]; m.p: 131.8-141.4° C.

### 6-[6-(2-Chlorophenyl)-8-cyclopropyl-7-oxo-7,8-dihydro-pyrido[2,3-d]pyrimidin-2-ylamino]-3-aza-bicyclo[3.1.0]hexane-3-carboxylic acid (4-fluorophenyl)-amide (Compound No. 26)

¹H NMR (CDCl₃): δ 8.45 (s, 1H, Ar—H), 7.51-7.45 (m, 2H, Ar—H), 7.36-7.28 (m, 5H, Ar—H), 7.01-6.96 (m, 2H, Ar—H), 6.11 (s, 1H, —NH), 5.65 (s, 1H, —NCH), 3.86 (d, 2H, J=9.0 Hz, —NCH₂), 3.67 (d, 2H, J=9.0 Hz, —NCH₂), 3.49 (s, 1H, —CH), 2.99 (s, 1H, —CH), 2.73 (s, 1H, —NH), 2.17-2.01 (m, 2H, —CH₂) and 1.29-1.27 (m, 2H, —CH₂); Mass spectrum (m/z, +ve ion mode): 532 [M⁺+1+2], 530 [M⁺+1]; m.p: 188.9-196.4° C.

### Example 8

### Synthesis of 6-(2-chlorophenyl)-8-cyclopropyl-2-(3-methyl-3-aza-bicyclo[3.1.0]hex-6-ylamino)-8H-pyrido[2,3-d]pyrimidin-7-one (Compound No. 20)

### Step a: 6-(2-Chlorophenyl)-8-cyclopropyl-2-methanesulphonyl-8H-pyrido[2,3-d]pyrimidin-7-one

The title compound was prepared following the procedure as described for the synthesis of 6-(2-chlorophenyl)-2-methanesulphonyl-8-methyl-8H-pyrido[2,3-d]pyrimidin-7-one by using cyclopropylamine in place of methylamine.

### Step b: 6-(2-chlorophenyl)-8-cyclopropyl-2-(3-methyl-3-aza-bicyclo[3.1.0]hex-6-ylamino)-8H-pyrido[2,3-d]pyrimidin-7-one

To a solution of the compound 6-(2-chlorophenyl)-8-cyclopropyl-2-methanesulphonyl-8H-pyrido[2,3-d]pyrimidin-7-one (0.1 g, 0.266 mmol) in pyridine (1 ml) was added 3-methyl-3-aza-bicyclo[3.1.0]hex-6-ylamine (0.0792 g, 0.533 mmol) and stirred for 12 hours at 80-100° C. Pyridine was removed azeotropically with toluene under reduced pressure and the residue thus obtained was purified by column chromatography using methanol in ethylacetate (1:19) solvent mixture as eluent to furnish the title compound. Yield=0.030 g.

¹H NMR (CDCl₃+CD₃OD): δ 8.42 (s, 1H, Ar—H), 7.53-7.33 (brm, 5H, Ar—H), 3.20 (d, 2H, J=9.0 Hz, —NCH₂), 3.08 (s, 1H, —NHCH), 2.55 (d, 2H, J=9.0 Hz, —NCH₂), 2.35 (s, 3H, —NCH₃), 1.96 (s, 1H, —NCH), 1.71 (s, 1H, —NCH), 1.30-1.26 (brm, 4H, 2×-CH₂); Mass spectrum (m/z, +ve ion mode): 410 [M⁺+1+2], 408 [M⁺+1]; m.p: 190-191° C.

### Example 9

### Synthesis of 2-(3-aza-bicyclo[3.1.0]hex-6-ylamino)-6-(2-chlorophenyl)-8-methyl-8H-pyrido[2,3-d]pyrimidin-7-one (Compound No. 5)

To a solution of the compound No. 6 (0.5 g, 1.0 mmol) in methanol (20 ml), was added potassium hydroxide solution (40%, 5 ml) in methanol and refluxed for 8 hours. The solvent was evaporated under reduced pressure and water was added into it. The compound was extracted with ethylacetate, dried over anhydrous sodium sulphate, filtered and concentrated under reduced pressure. The residue thus obtained was purified by washing with hexane. Yield=0.30 g.

¹H NMR (CDCl₃+CD₃OD): δ 8.47 (s, 1H, Ar—H), 7.58 (s, 1H, Ar—H), 7.46 (m, 1H, Ar—H), 7.35-7.32 (m, 4H, 3Ar—H & —NH), 3.76 (s, 3H, —NCH₃), 3.71-3.38 (brm, 2H, —NCH₂), 3.11 (brs, 3H, —NCH₂ & —NCH), 2.79 (s, 1H, —NH) and 1.80 (brs, 2H, 2×-CH); Mass spectrum (m/z, +ve ion mode): 370 [M⁺+1+2], 368 [M⁺+1]; m.p: 165-169.9° C.

The analogues of 2-(3-aza-bicyclo[3.1.0]hex-6-ylamino)-6-(2-chlorophenyl)-8-methyl-8H-pyrido[2,3-d]pyrimidin-7-(Compound No. 5) described below, can be prepared by deprotecting appropriate amine in place of compound No. 6, respectively, as applicable in each case.

### 2-(3-Aza-bicyclo[3.1.0]hex-6-ylamino)-6-(2-chlorophenyl)-8-(4-fluorophenyl)-8H-pyrido[2,3-d]pyrimidin-7-one (Compound No. 13)

¹H NMR (CDCl₃): δ 8.56-8.52 (brs, 1H, Ar—H), 7.72-7.71 (s, 1H, Ar—H), 7.49-7.21 (m, 8H, Ar—H), 3.39-3.21 (m, 2H, —NCH₂), 2.50 (brs, 3H, —NCH₂ & —NCH) and 1.71 (brs, 2H, 2×-CH).

Mass spectrum (m/z, +ve ion mode): 450 [M⁺+1+2], 448 [M⁺+1]; m.p: 163.5-169.9° C.

### 2-(3-Aza-bicyclo[3.1.0]hex-6-ylamino)-6-(2-chlorophenyl)-8-cyclopropyl-8H-pyrido[2,3-d]pyrimidin-7-one (Compound No. 19)

¹H NMR (CDCl₃): δ 8.50 (s, 1H, Ar—H), 7.50-7.29 (m, 7H, 6Ar—H & NH), 5.56 (1H, brs, —NCH), 3.27-3.20 (d, 2H, —NCH₂), 3.06-3.02 (brm, 3H, —NCH₂ & —CHNH), 2.68 (s, 1H, —CH), 1.83 (brm, 3H, —CH₂ & —CH) and 1.31-1.30 (m, 2H, —CH₂); Mass spectrum (m/z, +ve in mode): 396 [M⁺+1+2], 394 [M⁺+1]; m.pt: 144.8-148.8° C.

### 2-(3-Aza-bicyclo[3.1.0]hex-6-ylamino)-6-(2-chlorophenyl)-8H-pyrido[2,3-d]pyrimidin-7-one (Compound No. 58)

¹H NMR (TFA-d): δ 9.02 (s, 1H, Ar—H), 8.06 (s, 1H, Ar—H), 7.63-7.42 (m, 4H, Ar—H), 7.63-7.42 (m, 4H, Ar—H), 4.04 (brs, 3H, —NCH₂ & —NCH), 3.24 (s, 1H, —CH), 2.53 (s, 2H, —NCH₂) and 2.31 (s, 1H, —CH); Mass spectrum (m/z, +ve ion mode): 356 [M⁺+1+2], 354 [M⁺+1]; m.p: 233.3-239.9° C.

### Example 10

### Synthesis of 2-(3-Acetyl-3-aza-bicyclo[3.1.0]hex-6-ylamino)-6-(2-chlorophenyl)-8-cyclopropyl-8H-pyrido[2,3-d]pyrimidin-7-one (Compound No. 23)

To a solution of the compound No. 19 (0.05 g, 0.1272 mmol) in pyridine (0.5 ml), was added acetic anhydride (0.0388 g, 0.3816 mmol) and stirred at room temperature for 2 hours. The reaction mixture was concentrated under reduced pressure and poured over crushed ice and stirred. The compound was extracted with ethylacetate, washed with water, dried over anhydrous sodium sulphate, filtered and evaporated under reduced pressure. The residue thus obtained was purified by adding into it minimum amount of dichloromethane and precipitating it by hexane. The solid thus obtained was filtered and dried. Yield=0.057 g

¹H NMR (CDCl₃): δ 8.43 (s, 1H, Ar—H), 7.51-7.32 (brm, 5H, Ar—H), 5.80 (brs, 1H, —NH), 3.99 (d, 1H, J=12.0 Hz, —NCH), 3.76-3.71 (brm, 2H, —NCH₂), 3.58-3.55 (brm, 1H, —NCH), 3.49 (brs, 1H, —NHCH), 2.98 (brm, 1H, —NCH), 2.63 (s, 1H, —CH), 2.06 (s, 3H, —C(═O)CH₃), 1.91-1.84 (brm, 3H, —CH₂& —NCH) and 1.42-1.27 (brm, 2H, —CH₂); Mass spectrum (m/z, +ve ion mode): 438 [M⁺+1+2], 436 [M⁺+1]; m.p: 153.2-168.8° C.

Following analogues were prepared similarly,

### 2-(3-Acetyl-3-aza-bicyclo[3.1.0]hex-6-ylamino)-6-(2-chlorophenyl)-8-methyl-8H-pyrido[2,3-d]pyrimidin-7-one (Compound No. 45)

Mass (m/z, +ve ion mode): 412 [M⁺+1+2], 410 [M⁺+1].

### 2-(3-Acetyl-3-aza-bicyclo[3.1.0]hex-6-ylamino)-6-(2-chlorophenyl)-8H-pyrido[2,3-d]pyrimidin-7-one (Compound No. 48)

¹H NMR (CDCl₃): δ 8.52 (s, 1H, Ar—H), 7.69 (s, 1H, Ar—H), 7.48-7.20 (m, 8H, Ar—H), 3.69-3.65 (m, 1H, —NCH), 3.35 (s, 2H, —NCH₂), 2.08-2.00 (m, 2H, —NCH₂), 1.96 (s, 3H, —COCH₃), 1.74 (s, 1H, —CH) and 1.61 (s, 1H, —NCH); Mass spectrum (m/z, +ve ion mode): 492 [M⁺+1+2], 490 [M⁺+1], m.p: 260.8-262.4° C.

### 2-(3-Acetyl-3-aza-bicyclo[3.1.0]hex-ylamine)-6-(2-chlorophenyl)-8H-pyrido[2,3-]pyrimidin-7-one (Compound No. 78)

¹H NMR (TFA-d): δ 9.03 (s, 1H, Ar—H), 8.08 (s, 1H, Ar—H), 7.64-7.43 (m, 4H, Ar—H), 4.43-4.30 (m, 3H, —NCH₂ & —NCH), 4.12-4.09 (m, 1H, —NCH), 2.93 (s, 1H, —NCH), 2.65 (s, 3H, —COCH₃) and 2.47 (s, 2H, 2×-CH); Mass (positive ion mode) m/z: 398 [M⁺+1+2], 396 [M⁺+1]; m.p: 302.5-304.6° C.

### Example 11

### Synthesis of 6-(2-chlorophenyl)-8-cyclopropyl-2-{3-[2-(1,1,3,3-tetramethyl-butylamino)-acetyl]-3-aza-bicyclo[3.1.0]hex-6-ylamino}-8H-pyrido[2,3-d]pyrimidin-7-one (Compound No. 24)

To a solution of the compound No. 19 (0.05 g, 0.127 mmol) in dichloromethane, was added triethylamine (0.054 ml, 0.382 mmol) and tert-octyl-isocyanate (0.040 g, 0.254 mmol) at 0° C. and stirred for 30 minutes at the same temperature. The reaction mixture was warmed to room temperature and stirred for additional 2 hours. The organic solvent was evaporated under reduced pressure and poured into water. The compound was extracted with ethylacetate, washed with water, dried over anhydrous sodium sulphate, filtered and evaporated under reduced pressure to furnish the title compound. Yield=0.075 g.

¹H NMR (TFA-d): δ 8.81 (s, 1H, Ar—H), 7.85 (s, 1H, Ar—H), 7.60-7.38 (brm, 4H, Ar—H), 4.24 (m, 2H, —NCH & —NHCH), 4.03-3.88 (brm, 4H, —NCH₂ & 2×-CH), 3.70 (d, 2H, J=9.0 Hz, —NCH₂), 3.17 (s, 3H, —CH₂—C—(CH₃)₂), 2.95-2.91 (m, 3H, —CH₂—C—(CH₃)₂), 2.33-2.29 (brs, 6H, 2×-CH₃) and 1.55-1.09 (brm, 9H, 3×-CH₂ & —CH₃); Mass spectrum (m/z, +ve ion mode): 551 [M⁺+1+2], 549 [M⁺+1]; m.p: 217.6-220° C.

Analogues of 6-(2-chlorophenyl)-8-cyclopropyl-2-{3-[2-(1,1,3,3-tetramethyl-butylamino)-acetyl]-3-aza-bicyclo[3.1.0]hex-6-ylamino}-8H-pyrido[2,3-d]pyrimidin-7-one (Compound No. 24) described below, can be prepared by reacting an amine with appropriate isocyanate in place of tert-octyl-isocyanate, respectively as applicable in each case.

### 6-[6-(2-Chlorophenyl)-8-methyl-7-oxo-7,8-dihydro-pyrido[2,3-d]pyrimidin-2-ylamino]-3-aza-bicyclo[3.1.0]hexane-3-carboxylic acid (4-fluorophenyl)-amide (Compound No. 2)

¹H NMR (CDCl₃): δ 8.48 (s, 1H, Ar—H), 7.56 (s, 1H, Ar—H), 7.46 (s, 2H, Ar—H), 7.33-7.26 (s, 7H, Ar—H & —NH), 6.98 (brs, 2H, Ar—H), 6.18 (s, 1H, —NH), 3.87-3.84 (d, 2H, J=9.0 Hz, —NCH₂), 3.77 (s, 3H, —NCH₃), 3.66-3.69 (d, 2H, J=9.0 Hz, —NCH₂), 2.70 (s, 1H, —NCH) and 1.94 (brs, 2H, 2×-CH); Mass spectrum (m/z, +ve ion mode): 507 [M⁺+1+2], 505 [M⁺+1].

m.p: 138-139° C.

### 6-[6-(2-Chlorophenyl)-8-methyl-7-oxo-7,8-dihydro-pyrido[2,3-d]pyrimidin-2-ylamino]-3-aza-bicyclo[3.1.0]hexane-3-carboxylic acid isopropylamide (Compound No. 3)

¹H NMR (CDCl₃+CD₃OD): δ 8.47 (s, 1H, Ar—H), 7.56-7.55 (d, 1H, J=3.0 Hz, Ar—H), 7.48-7.46 (s, 1H, Ar—H), 7.33-7.27 (m, 4H, 3Ar—H & —NH), 3.99 (s, 3H, —NCH₃) 3.84-3.69 (m, 3H, —NCH & —NCH₂), 3.54-3.50 (d, 2H, J=9.0 Hz, —NCH₂), 2.65 (m, 2H, 2×-CH) and 1.40-1.12 (m, 6H, 2×-CH₃); m.p: 156-157° C.

### 6-[6-(2-Chloro-phenyl)-8-cyclopropyl-7-oxo-7,8-dihydro-pyrido[2,3-d]pyrimidin-2-ylamino]-3-aza-bicyclo[3.1.0]hexane-3-carboxylic acid isopropylamide (Compound No. 29)

¹H NMR (CDCl₃+CD₃OD): δ 8.54 (1H, Ar—H), 7.82 (s, 1H, Ar—H), 7.68 (s, 1H, Ar—H), 7.49-7.35 (brm, 5H, 3Ar—H & 2×—NH), 3.91-3.89 (m, 1H, —NCH), 3.78-3.75 (m, 3H, —NCH₂ & —NCH), 3.49-3.46 (3H, m, —NCH₂ & —NCH), 3.00 (m, 1H, —CH), 2.58 (m, 1H, —CH), 1.33-1.28 (m, 2H, —CH₂), 1.16-1.4 (m, 6H, 2×-CH₃) and 0.94-0.89 (m, 2H, —CH₂); Mass spectrum (m/z, +ve ion mode): 481 [M⁺+1+2] and 479 [M⁺+1]; m.p: 164.6-167° C.

### 6-[6-(2-Chlorophenyl)-8-cyclopropyl-7-oxo-7,8-dihydro-pyrido[2,3-d]pyrimidin-2-ylamino]-3-aza-bicyclo[3.1.0]hexane-3-carbothioic acid isopropylamide (Compound No. 30)

¹H NMR (CDCl₃): δ 8.43 (s, 1H, Ar—H), 7.50-7.1 (brm, 7H, 5Ar—H & 2×—NH), 5.62 (brm, 1H, —NCH), 4.63-4.61 (brm, 1H, —NCH), 4.00-3.97 (brm, 2H, —NCH₂), 3.73-3.70 (brm, 2H, —NCH₂), 3.53 (brm, 1H, —NCH), 2.96 (brm, 1H, —CH), 2.66-2.62 (brm, 1H, —CH), 1.97 (brm, 2H, —CH), 1.55 (brm, 6H, 2×-CH₃) and 0.95-0.85 (brm, 2H, —CH₂); Mass spectrum (m/z, +ve ion mode): 497 [M⁺+1+2], 495 [M⁺+1]; m.p: 184.4-200.6° C.

### 6-[6-(2-Chlorophenyl)-8-methyl-7-oxo-7,8-dihydro-pyrido[2,3-d]pyrimidin-2-ylamino]-3-aza-bicyclo[3.1.0]hexane-3-carboxylic acid (1,1,3,3-tetramethylbutyl)-amide (Compound No. 46)

¹H NMR (CDCl₃): δ 8.48 (s, 1H, Ar—H), 7.49-7.46 (m, 1H, Ar—H), 7.37-7.33 (m, 4H, Ar—H), 3.68 (s, 3H, —NCH₃), 3.51-3.47 (m, 2H, —NCH₂), 3.37-3.35 (m, 2H, —NCH₂), 2.61 (s, 1H, —NCH), 1.72 (s, 2H, 2×-CH), 1.41 (s, 6H, 2×-CH₃), 1.25 (s, 2H, —CH₂) and 1.01 (s, 9H, 3×-CH₃);

Mass (m/z, +ve ion mode): 525 [M⁺+1+2], 523 [M⁺+1]; m.p: 196.5-199.6° C.

### Example 12

### Synthesis of 6-(2-chlorophenyl)-8-cyclopropyl-2-[3-(toluene-4-sulphonyl)-3-aza-bicyclo[3.1.0]hex-6-ylamino]-8H-pyrido[2,3-d]pyrimidin-7-one (Compound No. 21)

To a solution of the compound No. 19 (0.05 g, 0.127 mmol) in dichloromethane (2 ml), was added triethylamine (0.054 ml, 0.38 mmol) followed by addition of p-toluenesulphonyl chloride (0.0727 g, 0.38 mmol) slowly into the reaction mixture at 0° C. The resulting reaction mixture was stirred at 0° C. for 30 minutes and then at room temperature for 2 hours. The solvent was evaporated under reduced pressure and to it was added a saturated solution of sodium bicarbonate and stirred for 15 minutes at room temperature. The solid thus separated was filtered and washed with hexane. The crude compound was purified by column chromatography using methanol in ethylacetate (1:9) solvent mixture as eluent to furnish the title compound. Yield=0.060 g.

¹H NMR (TFA-d): δ 8.75 (s, 1H, Ar—H), 7.84-7.81 (m, 3H, Ar—H), 7.57-7.35 (brm, 6H, Ar—H), 4.34-4.32 (brm, 3H, —NCH₂ & —NCH), 3.40 (d, 2H, J=9.0 Hz, —NCH₂), 3.23 (m, 3H, 2×-CH & —NCH), 2.53 (s, 3H, Ar—CH₃), 2.11 (brm, 1H, —NCH₂), 1.51-1.48 (brm, 2H, —CH₂) and 1.41 (m, 2H, —CH₂); Mass spectrum (m/z, positive in mode): 550 [M⁺+1+2], 548 [M⁺+1]; m.p: 264.9-274° C.

The analogues of 6-(2-chlorophenyl)-8-cyclopropyl-2-[3-(toluene-4-sulphonyl)-3-aza-bicyclo[3.1.0]hex-6-ylamino]-8H-pyrido[2,3-d]pyrimidin-7-one (Compound No. 21) described below, can be prepared by reacting appropriate amine with a sulphonyl group in place of p-toluenesulphonyl chloride, respectively, as applicable in each case.

### 6-(2-Chlorophenyl-2-(3-methanesulphonyl-3-aza-bicyclo[3.1.0]hex-6-ylamino)-8-methyl-8H-pyrido[2,3-d]pyrimidin-7-one (Compound No. 4)

¹H NMR (CDCl₃+MeOD): δ 8.46 (s, 1H, Ar—H), 7.59 (s, 1H, Ar—H), 7.50-7.46 (m, 1H, Ar—H), 7.35-7.33 (m, 4H, 3Ar—H & —NH), 3.78-3.72 (m, 5H, —NCH₃ & —NCH₂), 3.50-3.46 (d, 2H, J=9.3 Hz, —NCH₂), 2.89 (brs, 4H, —SO₂CH₃ & —NCH), 1.92 (brs, 2H, 2×-CH); Mass spectrum (m/z, +ve in mode): 447 [M⁺+1+2], 445 [M⁺+1]; m.p: 268-269° C.

### 6-(2-Chlorophenyl)-8-(4-fluorophenyl)-2-(3-methanesulphonyl)-3-aza-bicyclo[3.1.0]hex-6-ylamino)-8H-pyrido[2,3-d]pyrimidin-7-one (Compound No. 11)

¹H NMR (CDCl₃): δ 8.51 (s, 1H, Ar—H), 7.68-7.67 (d, 1H, J=3.0 Hz, Ar—H), 7.46-7.25 (m, 9H, Ar—H), 3.63 (s, 3H, —CH₃), 3.22 (brs, 2H, —NCH₂), 2.78 (brs, 2H, —NCH₂), 2.18 (s, 1H, —NCH) and 1.63 (brs, 2H, 2×-CH); Mass spectrum (m/z, positive in mode): 528 [M⁺+1+2], 526 [M⁺+1]; m.p: 253.9-265.3° C.

### 6-(2-Chlorophenyl)-8-(4-fluorophenyl)-2-[3-(toluene-4-sulphonyl)-3-aza-bicyclo[3.1.0]hex-6-ylamino]-8H-pyrido[2,3-d]pyrimidin-7-one (Compound No. 15)

¹H NMR (TFA-d): δ 8.79 (s, 1H, Ar—H), 7.86-7.01 (m, 3H, Ar—H), 7.34-7.58 (m, 6H, Ar—H), 4.02-3.93 (m, 5H, —NCH₃ & —NCH₂), 3.93-3.37 (d, 2H, J=6.0 Hz, —NCH₂), 3.25-3.16 (d, 1H, —NCH), 2.53 (s, 3H, —CH₃) and 2.09 (brs, 2H, 2×-CH); Mass spectrum (m/z, positive in mode): 524 [M⁺+1+2], 522 [M⁺+1]; m.p: 297.3-299.6° C.

### 6-(2-Chlorophenyl)-8-cyclopropyl-2-(3-methanesulphonyl-3-aza-bicyclo[3.1.0]hex-6-ylamino)-8H-pyrido[2,3-d]pyrimidin-7-one (Compound No. 22)

¹H NMR (TFA-d): δ 8.81 (s, 1H, Ar—H), 7.85 (s, 1H, Ar—H), 7.57-7.40 (brm, 4H, Ar—H), 4.06-3.95 (brm, 4H, 2×-NCH₂), 3.27 (m, 2H, —CH & —NCH), 3.00 (s, 1H, —CH), 2.42 (s, 1H, —NCH₂), 1.51-1.40 (s, 3H, —SO₂CH₃) and 1.35 (brs, 4H, 2×-CH₂); Mass spectrum (m/z, =ve ion mode): 474 [M⁺+1+2], 472 [M⁺+1]; m.p: 261.3-275.3° C.

### 2-(3-Benzenesulphonyl-3-aza-bicyclo[3.1.0]hex-6-ylamino)-6-(2-chlorophenyl)-8-cyclopropyl-8H-pyrido[2,3-d]pyrimidin-7-one (Compound No. 31)

¹H NMR (CDCl₃): δ 8.44 (s, 1H, Ar—H), 7.80-7.97 (m, 2H, Ar—H), 7.58-7.34 (brm, 8H, Ar—H), 3.93-3.90 (m, 1H, —NCH), 3.83-3.79 (m, 4H, 2×-CH₂), 3.65-3.62 (brm, 2H, 2×-CH), 3.08 (brm, 1H, —NCH), 2.10-2.03 (brm, 2H, —CH₂) and 1.03-0.96 (brm, 2H, —CH₂); Mass spectrum (m/z, +ve ion mode): 533 [M⁺+1+2], 531 [M⁺+1]; m.p: 140.7-142.5° C.

### 6-(2-Chlorophenyl)-8-cyclopropyl-2-[3-(thiophene-2-sulphonyl)-3-aza-bicyclo[3.1.0]hex-6-ylamino]-8H-pyrido[2,3-d]pyrimidin-7-one (Compound No. 36)

¹H NMR (TFA-d): δ 9.18 (s, 1H, Ar—H), 8.27-8.24 (s, 3H, Ar—H), 8.00-7.72 (b, 5H, Ar—H), 4.46-4.35 (m, 2H, —NCH₂), 3.94-3.91 (m, 2H, —NCH₂), 3.69-3.62 (m, 2H, 2×-NCH), 2.64-2.58 (m, 2H, 2×-CH), 1.96 (m, 2H, —CH₂) and 1.58 (m, 2H, —CH₂); Mass spectrum (m/z, +ve ion mode): 542 [M⁺+1+2], 540 [M⁺+1]; m.p: 284.9-286.1° C.

### 6-(2-Chlorophenyl)-8-cyclopropyl-2-[3-(4-trifluoromethylbenzenesulfonyl-3-aza-bicyclo[3.1.0]hex-6-ylamino]-8H-pyrido[2,3-d]pyrimidin-7-one (Compound No. 37)

¹H NMR (TFA-d): δ 9.18 (s, 1H, Ar—H), 8.54-8.24 (m, 5H, Ar—H), 8.00-7.79 (m, 4H, Ar—H), 4.45-4.42 (m, 2H, —NCH₂), 4.35 (m, 1H, —NCH), 3.85-3.83 (m, 1H, —NCH), 3.69 (brm, 2H, —NCH₂), 2.64-2.57 (m, 2H, 2×-CH), 1.96-1.95 (m, 2H, —CH₂) and 1.58 (m, 2H, —CH₂); Mass spectrum (m/z, +ve ion mode): 604 [M⁺+1+2], 602 [M⁺+1]; m.p: 221.7° C.-222.7° C.

### 6-{2-Chlorophenyl)-8-cyclopropyl-2-[3-(4-ethoxybenznesulfonyl)-3-aza-bicyclo[3.1.0]hex-6-ylamino]-8H-pyrido[2,3-d]pyrimidin-7-one (Compound No. 38)

¹H NMR (TFA-d): δ 9.17 (s, 1H, Ar—H), 8.36-8.33 (d, 2H, J=9.0 Hz, Ar—H), 8.24 (s, 1H, Ar—H), 8.00-7.79 (m, 4H, Ar—H), 7.69-7.66 (d, 2H, J=9.0 Hz, Ar—H), 4.55 (s, 3H, —OCH₃), 4.47-4.35 (m, 3H, —NCH₂ & —NCH), 3.85-3.82 (m, 1H, —NCH), 3.67 (m, 2H, —NCH₂), 2.64-2.55 (m, 2H, 2×-CH), 1.58 (brs, 2H, —CH₂) and 0.99-0.96 (brs, 2H, —CH₂); Mass spectrum (m/z, +ve ion mode): 566 [M⁺+1+2], 564 [M⁺+1]; m.p: 233.1-234.2° C.

### 6-(2-Chlorophenyl)-8-cyclopropyl-2-(3-ethanesulfonyl-3-aza-bicyclo[3.1.0]hex-6-ylamino)-8H-pyrido[2,3-d]pyrimidin-7-one (Compound No. 39)

¹H NMR (TFA-d): δ 9.25 (s, 1H, Ar—H), 8.29 (s, 1H, Ar—H), 8.04-7.82 (m, 5H, 4Ar—H & —NH), 4.47-4.39 (m, 2H, —CH₂SO₂), 4.21 (m, 2H, —NCH₂), 4.18 (m, 2H, —NCH₂), 3.81-3.76 (m, 1H, —NCH), 3.39-3.35 (m, 1H, —NCH), 2.77-2.72 (m, 2H, 2×-CH), 2.01-1.82 (brm, 4H, 2×-CH₂) and 1.03-0.99 (m, 3H, —CH₃); Mass spectrum (m/z, +ve ion mode): 488 [M⁺+1+2], 486 [M⁺+1]; m.p: 226-227° C.

### 6-(2-Chloro-phenyl)-8-methyl-2-[3-(thiophene-2-sulfonyl)-3-aza-bicyclo[3.1.0]hex-6-ylamino]-8H-pyrido[2,3-d]pyrimidin-7-one (Compound No. 40)

¹H NMR (CDCl₃+CD₃OD): δ 8.50 (s, 1H, Ar—H), 7.73-7.71 (brm, 1H, Ar—H), 7.62 (brs, 2H Ar—H), 7.47 (brs, 2H, Ar—H) and 7.34 (brs, 3H, Ar—H), 7.21 (brs, 1H, —NH), 3.35 (brm, 7H, 2×-NCH₂ & —NCH₃), 2.71 (brs, 2H, —NCH) and 1.84 (brm, 2H, 2×-CH); Mass spectrum (m/z, +ve ion mode): 516 [M⁺+1+2], 514 [M⁺+1]; m.p: 282-283° C.

### 2-(3-Benzenesulphonyl-3-aza-bicyclo[3.1.0]hex-6-ylamino)-6-(2-chlorophenyl)-8-methyl-8H-pyrido[2,3-d]pyrimidin-7-one (Compound No. 41)

¹H NMR (CDCl₃+CD₃OD): δ 8.48 (s, 1H, Ar—H), 7.85-7.83 (m, 2H, Ar—H), 7.69-7.57 (brm, 4H, Ar—H), 7.50-7.33 (brm, 5H, 4Ar—H & —NH), 3.80-3.71 (m, 5H, —NCH₃ & —NCH₂), 3.86 (m, 2H, —NCH₂), 3.24-3.22 (m, 2H, —NCH & —CH) and 2.77 (m, 1H, —CH); Mass spectrum (m/z, +ve ion mode): 510 [M⁺+1+2], 508 [M⁺+1]; m.p: 270-271° C.

### 6-(2-Chlorophenyl)-2-(3-ethanesulfonyl-3-aza-bicyclo[3.1.0]hex-6-ylamino)-8-methyl-8H-pyrido[2,3-d]pyrimidin-7-one (Compound No. 42)

¹H NMR (CDCl₃+CD₃OD): δ 8.49 (s, 1H, Ar—H), 7.60 (s, 1H, Ar—H), 7.47-7.33 (brm, 5H, 4-Ar—H & —NH), 3.79-3.76 (m, 2H, —CH₂SO₂), 3.57-3.54 (m, 2H, —NCH₂), 3.40-3.36 (m, 3H, —NCH₃), 3.10-3.03 (m, 2H, —NCH₂), 2.84 (m, 1H, —NCH), 2.03 (m, 2H, 2×-CH) and 1.41-1.36 (m, 3H, —CH₃); Mass spectrum (m/z, +ve ion mode): 462 [M⁺+1+2], 460 [M⁺+1]; m.p: 267-268° C.

### 6-(2-Chlorophenyl)-8-methyl-2-[3-(toluene-4-sulfonyl)-3-aza-bicyclo[3.1.0]hex-6-ylamino]-8H-pyrido[2,3-d]pyrimidin-7-one (Compound No. 43)

¹H NMR (TFA-d): δ 8.79 (s, 1H, Ar—H), 7.86-7.81 (m, 3H, Ar—H), 7.58-7.34 (m, 6H, Ar—H), 4.02 (s, 3H, —NCH₃), 3.96-3.93 (m, 2H, —NCH₂), 3.39-3.37 (m, 2H, —NCH₂), 3.16 (s, 1H, —NCH), 2.53 (s, 3H, Ar—CH₃), 2.09 (s, 2H, 2×-CH); Mass (m/z, +ve ion mode): 524 [M⁺+1+2], 522 [M⁺+1]; m.p: 297.3-299.6° C.

### 6-(2-Chlorophenyl)-2-[3-(4-methoxybenzenesulfonyl)-3-aza-bicyclo[3.1.0]hex-6-ylamino]-8-methyl-8H-pyrido[2,3-d]pyrimidin-7-one (Compound No. 47)

¹H NMR (TFA-d): δ 9.26 (s, 1H, Ar—H), 8.38-8.32 (m, 3H, Ar—H), 8.03-7.80 (m, 4H, Ar—H), 7.71-7.68 (m, 2H, Ar—H), 4.57 (s, 3H, —OCH₃), 4.38 (s, 3H, —NCH₃), 3.85-3.83 (m, 2H, N—CH₂), 3.61 (s, 1H, —NCH), 2.56 (s, 2H, —NCH₂), 2.14 (s, 1H, —CH) and 1.81 (s, 1H, —CH); Mass spectrum (m/z, +ve ion mode): 540 [M⁺+1+2], 538 [M⁺+1]; m.p: 254.1-255.8° C.

### Example 13

### Synthesis of N-(3-benzoyl-3-aza-bicyclo[3.1.0]hex-6-yl)-N-[6-(2-chlorophenyl)-8-cyclopropyl-7-oxo-7,8-dihydro-pyrido[2,3-d]pyrimidin-2-yl]-benzamide (Compound No. 25)

The title compound was prepared following the procedure as described for the synthesis of Compound No. 21 by using benzoyl chloride in place of p-toluene sulphonylchloride. Yield: 0.040 g.

¹HNMR (CDCl₃): δ 8.57 (s, 1H, Ar—H), 7.55-7.30 (m, 15H, Ar—H), 4.45-4.41 (brm, 1H, —NCH), 3.82-3.69 (brm, 3H, —NCH₂ & —NCH), 2.87 (s, 1H, —NH), 2.57-2.53 (brm, 1H, —N(CH)C(═O)), 2.30 (s, 1H, —CH), 2.16 (s, 1H, —CH), 1.55 (m, 2H, —CH₂) and 1.26 (m, 2H, —CH₂); Mass spectrum (m/z, +ve ion mode): 604 [M⁺+1+2], 602 [M⁺+1]; m.p: 124.5-129.8° C.

Following analogues were prepared similarly,

### 2-(3-Benzoyl-3-aza-bicyclo[3.1.0]hex-6-ylamino)-6-(2-chlorophenyl)-8-cyclopropyl-8H-pyrido[2,3-d]pyrimidin-7-one (Compound No. 28)

¹H NMR (CDCl₃): δ 8.42 (s, 1H, Ar—H), 7.50-7.32 (brm, 11H, 10 Ar—H & —NH), 4.40-4.36 (m, 1H, —NCH), 3.73-3.71 (brm, 5H, 2×-NCH₂ & —NCH), 2.92 (m, 1H, —CH), 2.65 (m, 1H, —CH), 1.92-1.83 (m, 2H, —CH₂) and 1.17-1.15 (m, 2H, —CH₂); Mass spectrum (m/z, +ve ion mode): 500 [M⁺+1+2], 498 [M⁺+1]; m.p: 216.5-222.2° C.

### 6-(2-Chlorophenyl)-8-methyl-2-[3-(4-methylbenzoyl)-3-aza-bicyclo[3.1.0]hex-6-ylamino]-8H-pyrido[2,3-d]pyrimidin-7-one (Compound No. 44)

¹H NMR (CDCl₃): δ 8.47 (s, 1H, Ar—H), 7.55 (s, 1H, Ar—H), 7.45-7.44 (m, 5H, Ar—H), 7.33-7.30 (m, 4H, Ar—H), 3.71 (s, 3H, —NCH₃), 3.64 (s, 4H, 2×-NCH₂), 2.63 (s, 1H, —NCH), 1.91-1.84 (m, 2H, 2×-CH); Mass spectrum (m/z, +ve ion mode): 474 [M⁺+1+2], 472 [M⁺+1]; m.p: 214-216.1° C.

### Example 14

### Synthesis of 6-[6-(2-Chlorophenyl)-8-cyclopropyl-7-oxo-7,8-dihydro-pyrido[2,3-d]pyrimidin-2-ylamino]-3-aza-bicyclo[3.1.0]hexane-3-carboxylic acid amide (Compound No. 63)

To a cooled solution of the compound 2-(3-Aza-bicyclo[3.1.0]hex-6-ylamino)-6-(2-chlorophenyl)-8-cyclopropyl-8H-pyrido[2,3-d]pyrimidin-7-one (0.05 g, 0.127 mmol) in dichloromethane (3 ml) was added triethyl amine (0.054 ml, 0.381 mmol) and stirred for 5 minutes. To the resulting reaction mixture was added trimethyl isocyanate (0.03 g, 0.254 mmol) and stirred at 0° C. for 30 minutes and then at room temperature for 2 hours. The mixture was diluted with water, extracted with ethyl acetate, washed with water, brine, dried over anhydrous sodium sulphate, filtered and evaporated under reduced pressure. The residue thus obtained was dissolved in dichloromethane followed by the addition of hexane. The solid thus separated was filtered and dried under reduced pressure to furnish the title compound. Yield: 0.020 g.

¹H NMR (CDCl₃): δ 8.42 (s, 1H, Ar—H), 7.50-7.29 (brm, 6H, 5Ar—H & —NH), 5.56 (s, 1H, —NH), 3.28-3.20 (m, 2H, —NCH₂), 3.11-3.02 (m, —NCH₂ & —NCH), 2.69-2.68 (m, 1H, —NCH) and 1.89-1.64 (m, 6H, 2×-CH₂ & 2×-CH); Mass spectrum (m/z, +ve ion mode): 439 [M⁺+1+2], 437 [M⁺+1].

### Example 15

### Synthesis of 3-{6-[6-(2-chloro-phenyl)-8-cyclopropyl-7-oxo-7,8-dihydro-pyrido[2,3-d]pyrimidin-2-ylamino]-3-aza-bicyclo[3.1.0]hex-3-yl}-propionitrile (Compound No. 64)

To a cooled solution of the compound 2-(3-Aza-bicyclo[3.1.0]hex-6-ylamino)-6-(2-chlorophenyl)-8-cyclopropyl-8H-pyrido[2,3-d]pyrimidin-7-one (0.10 g, 0.254 mmol) in methanol (5 ml) was added acrylonitrile (0.054 g, 1.02 mmol) dropwise and stirred at 0° C. for 10 minutes. The reaction mixture was brought to room temperature and then refluxed for 30 minutes. The organic solvent was evaporated under reduced pressure and the residue thus obtained was purified by column chromatography using ethyl acetate as an eluent to furnish the title compound. Yield: 0.040 g.

¹H NMR (CDCl₃): δ 8.41 (s, 1H, Ar—H), 7.54-7.32 (brm, 6H, 5Ar—H & —NH), 3.76 (brm, 2H, —NCH₂), 3.55 (brm, 1H, —NCH), 3.31 (brm, 3H, —NCH₂ & —NCH), 3.04 (brm, 2H, —NCH₂), 2.72 (brm, 2H, 2×-CH), 1.93 (m, 2H, —CH₂CN), 1.38-1.36 (m, 2H, —CH₂) and 1.03 (m, 2H, —CH₂); Mass spectrum (m/z, +ve ion mode): 449 [M⁺+1+2], 447 [M⁺+1]; m.p: 150.2-152.5° C.

Following analogues were prepared similarly,

### 3-{6-[6-{2-Chlorophenyl-8-methyl-7-oxo-7,8-dihydro-pyrido[2,3-d]pyrimidin-2-ylamino]-3-aza-bicyclo[3.1.0]hex-3-yl}-propionitrile (Compound No. 67)

¹H NMR (CDCl₃): δ 8.48 (s, 1H, Ar—H), 7.59 (s, 1H, Ar—H), 7.50-7.34 (brm, 5H, 4Ar—H & —NH), 4.08-3.69 (brm, 5H, —NCH₃ & —NCH₂), 3.37-3.27 (brm, 4H, 2×-NCH₂), 3.11 (brs, 1H, —NCH), 2.84-2.80 (brm, 2H, 2×-CH) and 2.61-2.54 (m, 2H, —CH₂); Mass spectrum (m/z, +ve ion mode): 423 [M⁺+1+2], 421 [M⁺+1]; m.p: 242-243° C.

### Example 16

### Synthesis of 2-(3-benzoyl-3-aza-bicyclo[3.1.0]hex-6-ylamino)-6-(2-chlorophenyl)-8-(4-fluorophenyl)-8H-pyrido[2,3-d]pyrimidin-7-one (Compound No. 49)

A mixture of the Compound No. 13 (0.070 g, 0.156 mmol), benzoic acid (0.02 g, 0.516 mmol) and hydroxy benzotriazole (0.032 g, 0.234 mmol) were evacuated for 15 minutes. Oxygen was flushed in the mixture followed by the addition of N-methylmorpholine (0.04 g, 0.391 mmol) and tetrahydrofuran (5 ml) at 0° C. and stirred at the same temperature for 1 hour. To the resulting reaction mixture was added 1-(3-dimethylaminopropyl)-3-ethylcarbodiimide hydrochloride (0.061 g, 0.313 mmol) at the same temperature and stirred at room temperature for 12 hours. The reaction mixture was poured into water, extracted with ethyl acetate, washed with water, dried over anhydrous sodium sulphate, filtered and evaporated under reduced pressure. The residue thus obtained was purified by column chromatography to furnish the title compound. Yield: 0.035 g.

¹H NMR (CDCl₃): δ 8.51 (s, 1H, Ar—H), 7.66 (s, 1H, Ar—H), 7.47-7.38 (m, 7H, Ar—H), 7.31-7.18 (m, 6H, Ar—H), 3.50-3.47 (m, 2H, —NCH₂), 2.69 (s, 1H, —NCH), 2.00 (s, 1H, —NCH), 1.73 (s, 1H, —NCH) and 1.60-1.56 (m, 2H, −2×-CH); Mass (m/z, +ve ion mode): 554 [M⁺+1+2], 552 [M⁺+1]; m.p: 242.4-244.4° C.

### Example 17

### Synthesis of Formula VIIIc

Step a, Procedure I: Synthesis of Formula VIIIa (when U′ is Hal-Substituted Alkyl)

To a solution of sodium hydride (60%, 0.014 g, 0.362 mmol) in N-methylpyrrolidinone (3 ml) at 0° C. was added the compound 6-(chlorophenyl)-2-methylsulfanyl-8H-pyrido[2,3-d]pyrimidin-8-yl]-7-one and stirred at room temperature for 1 hour. To the resulting reaction mixture was added hal-substituted alkyl (0.043 g, 0.362 mmol) at 0° C. and stirred at room temperature for 2 hours. The reaction mixture was poured into water and extracted with ethyl acetate, dried over anhydrous sodium sulphate, filtered and evaporated under reduced pressure. The residue thus obtained was purified by column chromatography using methanol in ethyl acetate as an eluent to furnish the title compound. Yield: 0.1 μg.

Step a: Procedure II: Synthesis of Compound of Formula VIIIa (when U′ is —OH(CH₂)ₖN(diethyl),

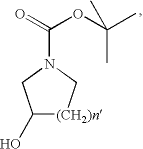

—OH(CH₂)ₖmorpholine or

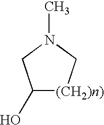

To a solution of triphenylphosphine (0.233 g, 0.988 mmol) in tetrahydrofuran (7 ml) was added 2-diethylamino ethanol (0.115 g, 0.988 mol) and stirred at room temperature for 10 minutes followed by the addition of the compound obtained from step a above (0.25 g, 0.823 mmol) and cooled to 0° C. To the resulting reaction mixture was added diisopropyl azodicarboxylate (0.199 g, 0.988 mmol) and stirred for 3 hours. The reaction mixture was poured into water, extracted with ethylacetate, washed with water and brine, dried over anhydrous sodium sulphate, filtered and evaporated under reduced pressure. The residue thus obtained was purified by column chromatography using ethylacetate in hexane as an eluent to furnish the title compound. Yield: 0.120 g.

### Step b, Procedure I: Synthesis of Compound of Formula VIIIb

The title compound was prepared following the procedure as described in Example 5 step e, by using compound obtained from step a above.

### Step c: Synthesis of Compound of Formula VIIIc

The title compound was prepared following the procedure as described in Example 5 step f, by condensing a compound obtained from step b, procedure I or step b, procedure II above with a compound of Formula X.

### Step d: Synthesis of Compound of Formula VIIId

To a solution of compound of Formula VIIIc (0.70 g, 1.435 mmol) in methanol (10 ml), was added methanolic potassium hydroxide (40%, 430.7 mmol) and refluxed for 6 hours. The reaction mixture was concentrated under vacuum and the crude product so obtained was poured into water. A pale yellow solid was obtained. It was then filtered and dried under vacuum. Yield: 0.40 g.

### Step e: Synthesis of Compound of Formula VIIIe

The title compound was prepared following the procedure as described in Example 10.

Following analogues were prepared similarly,

### 6-[6-(2-Chlorophenyl)-8-cyanomethyl-7-oxo-7,8-dihydro-pyrido[2,3-d]pyrimidin-2-ylamino]-3-aza-bicyclo[3.1.0]hexane-3-carboxylic acid (4-fluorophenyl)-amide (Compound No. 57)

¹H NMR (CDCl₃): δ 8.78 (s, 1H, Ar—H), 8.53 (s, 1H, Ar—H), 8.26 (s, 1H, Ar—H), 7.97 (s, 1H, Ar—H), 7.61-7.47 (m, 5H, Ar—H), 7.14-7.08 (m, 1H, Ar—H), 5.40 (s, 2H, —CH₂CN), 3.66-3.51 (m, 2H, —NCH₂), 2.75 (s, 1H, —NCH), 1.96 (s, 2H, —NCH₂) and 1.29 (s, 2H);

Mass spectrum (m/z, +ve ion mode): 532 [M⁺+1+2], 530 [M⁺+1]; m.p: 122-132.1° C.

### 2-(3-Acetyl-3-aza-bicyclo[3.1.0]hex-6-ylamino)-6-(2-chlorophenyl)-8-(2-diethylamino-ethyl)-8H-pyrido[2,3-d]pyrimidin-7-one (Compound No. 70)

¹H NMR (CDCl₃): δ 8.48 (s, 1H, Ar—H), 7.55 (s, 1H, Ar—H), 7.48-7.27 (m, 4H, Ar—H), 5.79 (s, 1H, —NH), 4.57 (m, 2H, —NCH₂), 3.99-3.57 (m, 4H), 2.84-2.79 (m, 2H, —NCH₂), 2.70-2.62 (m, 6H), 2.05-1.88 (s, 3H, —COCH₃), 1.56 (s, 2H), 1.25 (brs, 1H) and 1.07-1.03 (m, 6H, —CH₂CH₃); Mass spectrum (m/z, +ve ion mode): 497 [M⁺+1+2], 495 [M⁺+1]; m.p: 170.3-173.5° C.

### 3-[2-(3-Benzyl-3-aza-bicyclo[3.1.0]hex-6-ylamino}-6-(2-chlorophenyl)-7-oxo-7H-pyrido[2,3-d]pyrimidin-8-yl]-pyrrolidine-1-carboxylic acid tert-butyl ester (Compound No. 71)

¹H NMR (TFA-d): δ 9.41-9.29 (m, 1H, Ar—H), 8.29-8.24 (s, 1H, Ar—H), 7.57-7.33 (m, 9H, Ar—H), 6.15-6.06 (m, 1H, —NCH), 4.45 (s, 2H, —CH₂Ar), 4.20-3.50 (m, 8H, 4×-NCH₂), 2.52-2.49 (m, 1H, —NHCH), 2.23 (m, 2H, —CH₂), 1.62 (s, 9H, 3×-CH₃) and 1.41-1.29 (m, 2H, 2×-CH); Mass spectrum (m/z, +ve ion mode): 615 [M⁺+1+2], 613 [M⁺+1], m.p: 83.7-85.2° C.

### 2-(3-Benzyl-3-aza-bicyclo[3.1.0]hex-6-ylamino)-6-(2-chlorophenyl)-8-(2-morpholin-4-yl-ethyl)-8H-pyrido[2,3-d]pyrimidin-7-one (Compound No. 72)

¹H NMR (TFA-d): δ 8.48 (brs, 1H, Ar—H), 7.52 (s, 1H, Ar—H), 7.48-7.44 (m, 1H, Ar—H), 7.37-7.24 (m, 8H, Ar—H), 4.60 (brs, 2H, —OCH₂), 3.72-3.69 (m, 2H, —OCH₂), 3.64-3.62 (m, 4H, 2×-NCH₂), 3.19-3.16 (m, 2H, —NCH₂), 2.77-2.73 (m, 2H, —NCH₂), 2.66-2.48 (m, 6H, 3×-NCH₂), 2.05-2.00 (m, 2H, 2×-CH), 1.27-1.25 (m, 2H); Mass spectrum (m/z, +ve ion mode): 559 [M⁺+1+2], 557 [M⁺+1]; m.p: 52-52.4° C.

### 2-(3-Benzyl-3-aza-bicyclo[3.1.0]hex-6-ylamino)-6-(2-chlorophenyl)-8-(2-diethylaminoethyl)-8H-pyrido[2,3-d]pyrimidin-7-one (Compound No. 73)

¹H NMR (CDCl₃): δ 8.48 (brs, 1H, Ar—H), 7.53 (s, 1H, Ar—H), 7.47-7.27 (m, 8H, Ar—H), 4.70-4.65 (m, 2H, —CH₂NCO), 3.64 (s, 2H, —CH₂Ar), 3.28-3.19 (m, 2H, —NCH₂), 3.04 (brs, 2H, —NCH₂), 2.80 (brs, 5H, 2×-NCH₂ & —NCH), 2.50 (brs, 2H, —NCH₂) and 1.25-1.18 (m, 8H, —CH₃ & 2×-CH).

Mass spectrum (m/z, +ve ion mode): 545 [M⁺+1+2], 543 [M⁺+1].

### 2-(3-Benzyl-3-aza-bicyclo[3.1.0]hex-6-ylamino}-6-(2-chlorophenyl)-8-(1-methyl-pyrrolidin-3-yl)-8H-pyrido[2,3-d]pyrimidin-7-one (Compound No. 74)

¹H NMR (CDCl₃): δ 8.49 (brs, 1H, Ar—H), 7.72 (s, 1H, Ar—H), 7.45-7.19 (m, 9H, Ar—H), 4.13 (brs, 1H, —CHNCO), 3.57 (s, 2H, —CH₂Ar), 3.04-3.01 (m, 1H, —NCH), 2.90 (brs, 2H, —NCH₂), 2.48 (brs, 3H, —NCH₃), 2.11-1.56 (m, 6H, 3×-NCH₂) and 1.19 (s, 4H, —CH₂ & 2×-CH).

Mass spectrum (m/z, +ve ion mode): 529 [M⁺+1+2], 527 [M⁺+1].

### Example 18

### Synthesis of [2-(3-Benzyl-3-aza-bicyclo[3.1.0]hex-6-ylamino)-6-(2-chlorophenyl)-7-oxo-7H-pyrido[2,3-d]pyrimidin-8-yl]-acetic acid ethyl ester (Compound No. 75)

The title compound was prepared following the procedure described in Example 17 (step a, procedure I), by N-alkylating Compound No. 27 with bromoacetic acid ethyl ester.

¹H NMR (TFA-d): δ 9.38 (s, 1H, Ar—H), 8.40 (s, 1H, Ar—H), 8.05-7.79 (m, 9H, Ar—H), 5.88-5.81 (m, 2H, —NCH₂CO), 4.97-4.71 (m, 4H, —OCH₂ & —CH₂Ar), 4.57-4.53 (m, 2H, —NCH₂), 4.27-4.23 (m, 2H, —NCH₂), 3.78 (m, 1H, —NCH), 2.99-2.90 (m, 2H, 2×-CH) and 1.78-1.75 (m, 3H, —CH₂CH₃); Mass spectrum (m/z, +ve ion mode): 532 [M⁺+1+2], 530 [M⁺+1]; m.p: 185.5-188.5° C.

Following analogues were prepared similarly,

### 2-[2-(3-Benzyl-3-aza-bicyclo[3.1.0]hex-6-ylamino)-6-(2-chloro-phenyl)-7-oxo-7H-pyrido[2,3-d]pyrimidin-8-yl]-acetamide (Compound No. 79)

¹H NMR (TFA-d): δ 9.36 (s, 1H, Ar—H), 8.39 (s, 1H, Ar—H), 8.03-7.79 (m, 9H, Ar—H), 5.94 (m, 2H, —NCH₂CO), 4.92 (s, 2H, —NCH₂Ph), 4.59-4.56 (m, 2H, —NCH₂), 4.23-4.19 (m, 2H, —NCH₂), 3.94 (brs, 1H, —NCH) and 2.89 (2H, 2×-CH); Mass spectrum (m/z, +ve ion mode): 501 (M⁺+1), 503 (M⁺+1+2); m.p: 176.3-179.7° C.

### Example 19

### Synthesis of [2-(3-Benzyl-3-aza-bicyclo[3.1.0]hex-6-ylamino)-6-(2-chlorophenyl)-7-oxo-7H-pyrido[2,3-d]pyrimidin-8-yl]-acetic acid (Compound No. 76)

To a solution of the Compound No. 75 (0.05 g, 0.089 mmol) in methanol (5 ml) was added aqueous sodium hydroxide (5 ml) and stirred at room temperature for 2 hours. To the resulting reaction mixture was added hydrochloric acid solution (20%) till the pH of the solution was adjusted to 4-5. The solid thus separated out was filtered and washed with water and dried under reduced pressure to furnish the title compound. Yield: 0.025 g.

¹H NMR (TFA-d): δ 8.90 (s, 1H, Ar—H), 7.93 (s, 1H, Ar—H), 7.56-7.38 (m, 9H, Ar—H), 5.49 (brs, 2H, —NCH₂CO), 4.49 (s, 2H, —CH₂Ar), 10-4.08 (m, 2H, —NCH₂), 3.77-3.75 (m, 2H, —NCH₂), 3.29-3.27 (m, 1H, —CHNH), 2.55-2.44 (m, 2H, 2×-CH); Mass spectrum (m/z, +ve ion mode): 504 [M⁺+1+2], 502 [M⁺+1]; m.p: 158.7-162.6° C.

### Example 20

### Synthesis of 2-(3-Benzyl-3-aza-bicyclo[3.1.0]hex-6-ylamino)-6-(2-chlorophenyl)-8-pyrrolidin-3-yl-8H-pyrido[2,3-d]pyrimidin-7-one (Compound No. 77)

The title compound was prepared following the procedure as described in Example 3 step d, by deprotecting Compound No. 71

Mass (m/z, +ve ion mode): 515 [M⁺+1+2], 513 [M⁺+1]; m.p: 203.4-206.1° C.

### Example 20

### Synthesis of Synthesis of [4-({6-[6-(2-Chlorophenyl)-8-methyl-7-oxo-7,8-dihydro-pyrido[2,3-d]pyrimidin-2-ylamino]-3-aza-bicyclo[3.1.0]hexane-3-carbonyl]-amino)-phenyl]-acetic acid (Compound No. 81)

### Step a: Synthesis of [4-({6-[6-(2-Chlorophenyl)-8-methyl-7-oxo-7,8-dihydro-pyrido[2,3-d]pyrimidin-2-ylamino]-3-aza-bicyclo[3.1.0]hexane-3-carbonyl]-amino)-phenyl]-acetic acid (Compound No. 80)

To a solution of the Compound No. 5 (0.1 g, 0.272 mmol) in dimethylsulphoxide (0.5 ml) cooled at 0° C. was added Hunig's base (0.36 g, 0.272 mmol) and stirred for 1 hour at room temperature. To the resulting reaction mixture was added (4-methoxycarbonylamino-phenyl)-acetic acid methyl ester (0.086 g, 0.299 mmol) in dimethylsulphoxide dropwise. The mixture was stirred at room temperature overnight. The reaction mixture was poured into water, extracted with ethyl acetate, washed with water, dried over anhydrous sodium sulphate, filtered and concentrated under reduced pressure. The residue thus obtained was purified by column chromatography using ethyl acetate in hexane to furnish the title compound. Yield: 0.082 g.

¹H NMR (CDCl₃): δ 8.48 (s, 1H, Ar—H), 7.56 (s, 1H, Ar—H), 7.49-7.46 (m, 1H, Ar—H), 7.38-7.19 (m, 7H, Ar—H), 3.88-3.85 (m, 2H, —CH₂CO), 3.77 (s, 3H, —OCH₃), 3.68 (s, 4H, 2×-NCH₂), 3.57 (s, 3H, —NCH₃), 2.69 (s, 1H, —NCH) and 1.94 (s, 2H, 2×-CH); Mass spectrum (m/z, +ve ion mode): 561 [M⁺+1+2], 559 [M⁺+1]; m.p: 189.9-196.6° C.

### Step b: Synthesis of [4-({6-[6-(2-Chlorophenyl)-8-methyl-7-oxo-7,8-dihydro-pyrido[2,3-d]pyrimidin-2-ylamino]-3-aza-bicyclo[3.1.0]hexane-3-carbonyl]-amino)-phenyl]-acetic acid (Compound No. 81)

The title compound was prepared following the procedure as described in Example 19, by hydrolysis the compound obtained from step a above.

¹H NMR (TFA-d): δ 9.30 (s, 1H, Ar—H), 8.39-8.34 (brs, 1H, Ar—H), 8.03-7.83 (m, 8H, Ar—H), 4.57-4.49 (m, 6H, —COCH₂ & 2×-NCH₂), 4.34 (s, 3H, —NCH₃), 2.88 (s, 1H, —NCH) and 1.81 (s, 2H, —CH₂); Mass (m/z, +ve ion mode): 547 [M⁺+1+2], 545 [M⁺+1]; m.p: 227.1-235.4° C.

### Example 21

### Synthesis of 2-(3-Benzyl-3-aza-bicyclo[3.1.0]hex-6-ylamino)-6-(2-chlorophenyl)-8-(1H-tetrazol-5-ylmethyl)-8H-pyrido[2,3-d]pyrimidin-7-one (Compound No. 62)

To a solution of the Compound No. 56 (0.10 g, 0.207 mmol) in toluene was added triethylamine hydrochloride salt (0.05 g) and refluxed overnight. The solvent was evaporated under reduced pressure followed by the addition of water. The mixture was extracted with ethylacetate, washed, dried over anhydrous sodium sulphate, filtered and concentrated under reduced pressure. The residue thus obtained was purified by column chromatography using ethyl acetate in hexane to furnish the title compound to furnish the title compound. Yield: 0.030 g.

Mass (m/z, +ve ion mode): 528 [M⁺+1+2], 526 [M⁺+1].

### Methodology

### p38 Inhibition Assays

### Inhibition of Phosphorylation of EGF Receptor Peptide

Method 1: This assay was carried out in the presence of 10 mM MgCl₂, 25 mM β-glycerophosphate, 10% glycerol and 100 mM HEPES buffer at pH 7.6. For a typical IC50 determination, a stock solution is prepared containing all of the above components and activated p38 (5 nM). The stock solution was aliquoted into vials. A fixed volume of DMSO or inhibitor in DMSO (final concentration of DMSO in reaction was 5%) was introduced to each vial, mixed and incubated for 15 minutes at room temperature.

EGF receptor peptide, KRELVEPLTPSGEAPNQALLR, a phosphoryl acceptor in p38-catalysed kinase reaction (1), was added to each vial to a final concentration of 200 μM. The kinase reaction was initiated with ATP (100 μm) and the vials were incubated at 30° C. After 30 minutes, the reactions were quenched with equal volume of 10% trifluoroacetic acid (TFA).

The phosphorylated peptide was quantified by HPLC analysis. Separation of the phosphorylated peptide from the unphosphorylated peptide was achieved on a reverse phase column (Deltapak, 5 μM, C18 100D, part no. 011795) with a binary gradient of water and acetonitrile, each containing 0.1% TFA. IC₅₀ (concentration of inhibitor yielding 50% inhibition) was determined by plotting the % activity remaining against inhibitor concentration. Compounds 1-21 were tested according to this method.

Method 2: p38 MAP Kinase inhibitory potential was evaluated utilizing the proprietary IQ technology (Pierce, Rockford, Ill.). The assay incorporates an iron-containing compound that binds specifically to phosphate groups present on fluorescent dye-labeled phosphorylated peptides which in this case was the Epidermal Growth Factor Receptor Peptide (KRELVEPLTPSGEAPNQALLR). Recombinant activated GST-p38MAP kinase-α (in house) was used at a concentration of 40 nM. The reaction was initiated with 100 μM ATP. When bound to the phosphate group, the iron-containing compound is brought into proximity to the fluorophore and act as a dark quencher of the fluorescent dye. Results were quantitated by comparing the observed relative fluorescence units of test samples to blanks containing no enzyme. A dose response curve was generated with different concentrations of inhibitor and the IC₅₀ was calculated using Graph Pad Prism. Compounds 22-81 were tested according to this method.

### Cell Based Assay for TNF-α Release

### Method of Isolation of Human Peripheral Blood Mononuclear Cells.

Human whole blood was collected in vacutainer tubes containing EDTA as an anti coagulant. A blood sample (7 ml) was carefully layered over 5 ml PMN Cell Isolation Medium (Robbins Scientific) in a 15 ml round bottom centrifuge tubes. The sample was centrifuged at 450-500×g for 30-35 minutes in a swing-out rotor at room temperature. After centrifugation the top band of cells were removed and washed 3 times with PBS w/o calcium or magnesium. The cells were centrifuged at 400×g for 10 minutes at room temperature. The cells were resuspended in Macrophage Serum Free Medium (Gibco BRL) at concentration of 2 million cells/ml.

The IC₅₀ values for TNF-α release from peripheral blood mononuclear cells for particular compounds provided herein (compound Nos. 2, 4, 6, 12, 16-20, 23-24, 26-27, 32-33, 35, 56-58 and 63) were found to range from about 2.3 μM to about 12 nM, for example from about 1.5 μM to about 12 nM, or from about 400 nM to about 12 nM, or from about 100 nM to about 12 nM.

### LPS Stimulation of Human PBMNC's:

PBM cells (0.1 ml; 2 million/ml) were co-incubated with 0.1 ml of compound (10-0.41 μM, final concentration) for 1 hour in flat bottom 96 well microtiter plate. Compounds were dissolved in DMSO initially and diluted in TCM for a final concentration of 0.1% DMSO. LPS (Cal biochem, 20 ng/ml, final concentration) was then added at volume of 0.010 ml. Cultures were incubated overnight at 37° C.). Supernatant were then removed and tested by ELISA for TNF-α release. Viability was analyzed using MTT. After 0.1 ml supernatant was collected, 0.1 ml of 0.25 mg/ml of MTT was added to remaining 0.1 ml of cells. The cells were incubated at 37° C. for 2-4 hours, then the O.D was measured at 490-650 nm n.

The TNF-α levels released in the culture medium were quantitated by ELISA. Inhibitory potency was expressed as IC₅₀.

The compounds 1 to 81 disclosed above showed p38 inhibitory activity in a range of from about 10 μM to about 25 nM, for example from about 900 nM to about 25 nM, or from about 400 nm to about 25 nM, or from about 60 nM to about 25 nM. A few of the compounds tested (Compound Nos. 15, 21, 24, 37, 41, 43 and 61) formed a precipitate in the DMSO solvent used.

1. Compounds having the structure of Formula I: 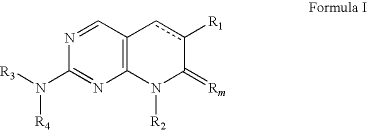
and its pharmaceutically acceptable salts, pharmaceutically acceptable solvates, esters, enantiomers diastereomers, N-oxides, polymorphs, metabolites;
wherein
R₁ is alkyl, alkenyl, alkynyl, cycloalkyl, aryl, aralkyl, heteroaryl, heterocyclyl, heteroarylalkyl, or heterocyclylalkyl;
when Rₘ is oxygen or sulphur, R₂ is alkyl, alkenyl, alkynyl, cycloalkyl, aryl, aralkyl, heteroaryl, heterocyclyl, heterocyclylalkyl or heteroarylalkyl;
when Rₘ is —NH, —N-acyl, —N(CN), —N(NO₂), —C(R₃)₂ or —CH(NO₂), R₂ is hydroxy, alkoxy, aryloxy, —CHO, —CN, alkyl, alkenyl, alkynyl, cycloalkyl, carboxy, halogen, aryl, aralkyl, acyl, heteroaryl, heterocyclyl, —SO₂R₅, —COOR₆, —C(═O)NRₓR_{y}, —NRₓR_{y} or —OC(═O)NRₓR_{y}, —NHC(═O)Rₓ;
the symbol [symbol] represents a single bond or a double bond;
R₃ is hydrogen, alkyl, alkenyl, alkynyl, cycloalkyl, aryl, heterocyclyl, heteroaryl, aralkyl, heteroarylalkyl or heterocyclylalkyl;
R₄ is 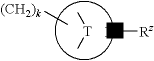
(wherein 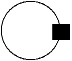
represents a cyclic ring having 4 or 5 carbon atoms, k is an integer selected from 0-2, M is O or N, and T is —(CH₂)ₙ—, —CH(O)CH₂—, —CH₂CH(O)CH₂—, —CH(O)—, —CH₂—O—CH₂—, —CH₂—NH—CH₂—);
R_{z} is no atom (when M is oxygen), hydrogen or Rᵤ (wherein Rᵤ is hydroxy, alkoxy, aryloxy, —CHO, —CN, alkyl, alkenyl, alkynyl, cycloalkyl, carboxy, halogen, aryl, aralkyl, acyl, heteroaryl, heterocyclyl, —SO₂R₅, —COOR₆, —C(═O)NRₓR_{y}, —NRₓR_{y} or —OC(═O)NRₓR_{y} or —NHC(═O)Rₓ);
n is an integer selected from 0-3 (wherein when n is zero then T represents a direct bond);
R₅ is alkyl, alkenyl, alkynyl, cycloalkyl, —NRₚR_{q} (wherein Rₚ and R_{q} is hydrogen, alkyl, alkenyl, alkynyl, cycloalkyl, aryl, aralkyl, heterocyclyl, heteroaryl, heterocyclylalkyl or heteroarylalkyl, or Rₚ and R_{q} may also together join to form a heterocyclyl ring), aryl, aralkyl, heteroaryl, heterocyclyl, heterocyclylalkyl or heteroarylalkyl;
R₆ is alkyl, alkenyl, alkynyl, cycloalkyl, aryl, aralkyl, heteroarylalkyl or heterocyclylalkyl;
Rₓ and R_{y} are independently hydrogen, alkyl, alkenyl, alkynyl, cycloalkyl, aryl, aralkyl, —SO₂R₅ (wherein R₅ is the same as defined above), heteroaryl, heterocyclyl, heteroarylalkyl or heterocyclylalkyl;
Q is alkyl, alkenyl, alkynyl, cycloalkyl, aryl, heteroaryl, heterocyclyl, aralkyl, heteroarylalkyl or heterocyclylalkyl.

2. A compound according to claim 1, wherein R₁ is optionally substituted aryl.

3. (canceled)

4. A compound according to claim 1, wherein R₂ is optionally substituted alkyl, cycloalkyl or aryl.

5. (canceled)

6. A compound according to claim 1, wherein R₃ is hydrogen, alkyl, alkenyl or alkynyl.

7. (canceled)

8. A compound according to claim 1, wherein Rₘ is oxygen, R₁ is optionally substituted aryl, R₂ is optionally substituted alkyl, cycloalkyl or aryl, R₃ is hydrogen and R₄ is

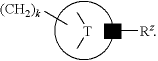

9. (canceled)

10. (canceled)

11. (canceled)

12. A compound according to claim 1, wherein T is —(CH₂)ₙ wherein n is 0 or 2 wherein when n is zero then T represents a direct bond.

13. A compound according to claim 1, wherein 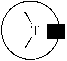
is azabicyclohexane or azabicycloctane.

14. A compound according to claim 1, wherein R_{z} is hydrogen or Rᵤ.

15. (canceled)

16. (canceled)

17. A compound selected from the group consisting of
2-(3-Benzyl-3-aza-bicyclo[3.1.0]hex-6-ylamino)-6-(2-chlorophenyl)-8-methyl-8H-pyrido[2,3-d]pyrimidin-7-one (Compound No. 1);
6-[6-(2-Chlorophenyl)-8-methyl-7-oxo-7,8-dihydro-pyrido[2,3-d]pyrimidin-2-ylamino]-3-aza-bicyclo[3.1.0]hexane-3-carboxylic acid-(4-fluorophenyl)-amide (Compound No. 2);
6-[6-(2-Chlorophenyl)-8-methyl-7-oxo-7,8-dihydro-pyrido[2,3-d]pyrimidin-2-ylamino]-3-aza-bicyclo[3.1.0]hexane-3-carboxylic acid isopropylamide (Compound No. 3);
6-(2-Chlorophenyl)-2-(3-methanesulphonyl-3-aza-bicyclo[3.1.0]hex-6-ylamino)-8-methyl-8H-pyrido[2,3-d]pyrimidin-7-one (Compound No. 4);
2-(3-Aza-bicyclo[3.1.0]hex-6-ylamino)-6-(2-chlorophenyl)-8-methyl-8H-pyrido[2,3-d]pyrimidin-7-one (Compound No. 5);
6-[6-(2-Chlorophenyl)-8-methyl-7-oxo-7,8-dihydro-pyrido[2,3-d]pyrimidin-2-ylamino]-3-aza-bicyclo[3.1.0]hexane-3-carboxylic acid benzyl ester (Compound No. 6);
2-(3-Benzyl-3-aza-bicyclo[3.2.1]oct-8-ylamino)-6-(2-chlorophenyl)-8-methyl-8H-pyrido[2,3-d]pyrimidin-7-one (Compound No. 7);
2-(3-Benzyl-3-aza-bicyclo[3.1.0]hex-6-ylamino)-6-(2-chlorophenyl)-8-(4-fluorophenyl)-8H-pyrido[2,3-d]pyrimidin-7-one (Compound No. 8);
2-(3-Benzyl-3-aza-bicyclo[3.2.1]oct-8-ylamino)-6-(2-chlorophenyl)-8-(4-fluorophenyl)-8H-pyrido[2,3-d]pyrimidin-7-one (Compound No. 9);
6-[6-(2-Chlorophenyl)-8-(4-fluorophenyl)-7-oxo-7,8-dihydro-pyrido[2,3-d]pyrimidin-2-ylamino]-3-aza-bicyclo[3.1.0]hexane-3-carboxylic acid (4-fluorophenyl)-amide (Compound No. 10);
6-(2-Chlorophenyl)-8-(4-fluorophenyl)-2-(3-methanesulphonyl-3-aza-bicyclo[3.1.0]hex-6-ylamino)-8H-pyrido[2,3-d]pyrimidin-7-one (Compound No. 11);
6-[6-(2-Chlorophenyl)-8-(4-fluorophenyl)-7-oxo-7,8-dihydro-pyrido[2,3-d]pyrimidin-2-ylamino]-3-aza-bicyclo[3.1.0]hexane-3-carboxylic acid benzyl ester (Compound No. 12);
2-(3-Aza-bicyclo[3.1.0]hex-6-ylamino)-6-(2-chlorophenyl)-8-(4-fluorophenyl)-8H-pyrido[2,3-d]pyrimidin-7-one (Compound No. 13);
6-[6-(2-Chlorophenyl)-8-(4-fluorophenyl)-7-oxo-7,8-dihydro-pyrido[2,3-d]pyrimidin-2-ylamino]-3-aza-bicyclo[3.1.0]hexane-3-carboxylic acid isopropylamide (Compound No. 14);
6-(2-Chlorophenyl)-8-(4-fluorophenyl)-2-[3-(toluene-4-sulphonyl)-3-aza-bicyclo[3.1.0]hex-6-ylamino]-8H-pyrido[2,3-d]pyrimidin-7-one (Compound No. 15);
2-[(3-Benzyl-3-aza-bicyclo[3.1.0]hex-6-ylmethyl)-amino]-6-(2-chlorophenyl)-8-(4-fluorophenyl)-8H-pyrido[2,3-d]pyrimidin-7-one (Compound No. 16);
2-(3-Benzyl-3-aza-bicyclo[3.1.0]hex-6-ylamino)-6-(2-chlorophenyl)-8-cyclopropyl-8H-pyrido[2,3-d]pyrimidin-7-one (Compound No. 17);
6-[6-(2-Chlorophenyl)-8-cyclopropyl-7-oxo-7,8-dihydro-pyrido[2,3-d]pyrimidin-2-ylamino]-3-aza-bicyclo[3.1.0]hexane-3-carboxylic acid benzyl ester (Compound No. 18);
2-(3-Aza-bicyclo[3.1.0]hex-6-ylamino)-6-(2-chlorophenyl)-8-cyclopropyl-8H-pyrido[2,3-d]pyrimidin-7-one (Compound No. 19);
6-(2-Chlorophenyl)-8-cyclopropyl-2-(3-methyl-3-aza-bicyclo[3.1.0]hex-6-ylamino)-8H-pyrido[2,3-d]pyrimidin-7-one (Compound No. 20);
6-(2-Chlorophenyl)-8-cyclopropyl-2-[3-(toluene-4-sulphonyl)-3-aza-bicyclo[3.1.0]hex-6-ylamino]-8H-pyrido[2,3-d]pyrimidin-7-one (Compound No. 21);
6-(2-Chlorophenyl)-8-cyclopropyl-2-(3-methanesulphonyl-3-aza-bicyclo[3.1.0]hex-6-ylamino)-8H-pyrido[2,3-d]pyrimidin-7-one (Compound No. 22);
2-(3-Acetyl-3-aza-bicyclo[3.1.0]hex-6-ylamino)-6-(2-chlorophenyl)-8-cyclopropyl-8H-pyrido[2,3-d]pyrimidin-7-one (Compound No. 23);
6-(2-Chlorophenyl)-8-cyclopropyl-2-{3-[2-(1,1,3,3-tetramethyl-butylamino)-acetyl]-3-aza-bicyclo[3.1.0]hex-6-ylamino}-8H-pyrido[2,3-d]pyrimidin-7-one (Compound No. 24);
N-(3-Benzoyl-3-aza-bicyclo[3.1.0]hex-6-yl)-N-[6-(2-chlorophenyl)-8-cyclopropyl-7-oxo-7,8-dihydro-pyrido[2,3-d]pyrimidin-2-yl]-benzamide (Compound No. 25);
6-[6-(2-Chlorophenyl)-8-cyclopropyl-7-oxo-7,8-dihydro-pyrido[2,3-d]pyrimidin-2-ylamino]-3-aza-bicyclo[3.1.0]hexane-3-carboxylic acid-(4-fluorophenyl)-amide (Compound No. 26);
2-(3-Benzyl-3-aza-bicyclo[3.1.0]hex-6-ylamino)-6-(2-chloro-phenyl)-8H-pyrido[2,3-d]pyrimidin-7-one (Compound No. 27);
2-(3-Benzoyl-3-aza-bicyclo[3.1.0]hex-6-ylamino}-6-(2-chloro-phenyl)-8-cyclopropyl-8H-pyrido[2,3-d]pyrimidin-7-one (Compound No. 28);
6-[6-(2-Chloro-phenyl)-8-cyclopropyl-7-oxo-7,8-dihydro-pyrido[2,3-d]pyrimidin-2-ylamino]-3-aza-bicyclo[3.1.0]hexane-3-carboxylic acid isopropylamide (Compound No. 29);
6-[6-(2-Chloro-phenyl)-8-cyclopropyl-7-oxo-7,8-dihydro-pyrido[2,3-d]pyrimidin-2-ylamino]-3-aza-bicyclo[3.1.0]hexane-3-carbothioic acid isopropylamide (Compound No. 30)
2-(3-Benzenesulphonyl-3-aza-bicyclo[3.1.0]hex-6-ylamino)-6-(2-chlorophenyl)-8-cyclopropyl-8H-pyrido[2,3-d]pyrimidin-7-one (Compound No. 31)
2-(1-Benzyl-piperidin-4-ylamino)-6-(2-chloro-phenyl)-8-cyclopropyl-8H-pyrido[2,3-d]pyrimidin-7-one (Compound No. 32);
2-(7-Benzyl-3-oxa-7-aza-bicyclo[3.3.1]non-9-ylamino)-6-(2-chloro-phenyl)-8-cyclopropyl-8H-pyrido[2,3-d]pyrimidin-7-one (Compound No. 33);
2-(9-Benzyl-9-aza-bicyclo[3.3.1]non-3-ylamino)-6-(2-chloro-phenyl)-8-cyclopropyl-8H-pyrido[2,3-d]pyrimidin-7-one (Compound No. 34);
2-{3-Benzyl-3-aza-bicyclo[3.1.0]hex-6-ylamino}-6-{2-chloro-phenyl)-8-cyclopropyl-8H-pyrido[2,3-d]pyrimidin-7-one (Compound No. 35);
6-(2-Chlorophenyl)-8-cyclopropyl-2-[3-(thiophene-2-sulphonyl)-3-aza-bicyclo[3.1.0]hex-6-ylamino]-8H-pyrido[2,3-d]pyrimidin-7-one (Compound No. 36);
6-(2-Chloro-phenyl)-8-cyclopropyl-2-[3-(4-trifluoromethyl-benzenesulfonyl)-3-aza-bicyclo[3.1.0]hex-6-ylamino]-8H-pyrido[2,3-d]pyrimidin-7-one (Compound No. 37);
6-{2-Chloro-phenyl)-8-cyclopropyl-2-[3-(4-ethoxy-benzenesulfonyl)-3-aza-bicyclo[3.1.0]hex-6-ylamino]-8H-pyrido[2,3-d]pyrimidin-7-one (Compound No. 38);
6-(2-chloro-phenyl)-8-cyclopropyl-2-(3-thanesulfonyl-3-aza-bicyclo[3.1.0]hex-6-ylamino)-8H-pyrido[2,3-d]pyrimidin-7-one (Compound No. 39);
6-{2-Chloro-phenyl}-8-methyl-2-[3-{thiophene-2-sulfonyl}-3-aza-bicyclo[3.1.0]hex-6-ylamino]-8H-pyrido[2,3-d]pyrimidin-7-one (Compound No. 40);
2-(3-Benzenesulphonyl-3-aza-bicyclo[3.1.0]hex-6-ylamino)-6-(2-chlorophenyl)-8-methyl-8H-pyrido[2,3-d]pyrimidin-7-one (Compound No. 41);
6-{2-Chloro-phenyl}-2-(3-ethanesulfonyl-3-aza-bicyclo[3.1.0]hex-6-ylamino)-8-methyl-8H-pyrido[2,3-d]pyrimidin-7-one (Compound No. 42);
6-(2-Chloro-phenyl)-8-methyl-2-[3-(toluene-4-sulfonyl)-3-aza-bicyclo[3.1.0]hex-6-ylamino]-8H-pyrido[2,3-d]pyrimidin-7-one (Compound No. 43);
6-(2-Chloro-phenyl)-8-methyl-2-[3-(4-methyl-benzoyl)-3-aza-bicyclo[3.1.0]hex-6-ylamino]-8H-pyrido[2,3-d]pyrimidin-7-one (Compound No. 44);
2-(3-Acetyl-3-aza-bicyclo[3.1.0]hex-6-ylamino)-6-(2-chloro-phenyl)-8-methyl-8H-pyrido[2,3-d]pyrimidin-7-one (Compound No. 45);
6-[6-(2-Chloro-phenyl)-8-methyl-7-oxo-7,8-dihydro-pyrido[2,3-d]pyrimidin-2-ylamino]-3-aza-bicyclo[3.1.0]hexane-3-carboxylic acid (1,1,3,3-tetramethyl-butyl)-amide (Compound No. 46);
6-(2-Chloro-phenyl)-2-[3-(4-methoxy-benzenesulfonyl)-3-aza-bicyclo[3.1.0]hex-6-ylamino]-8-methyl-8H-pyrido[2,3-d]pyrimidin-7-one (Compound No. 47);
2-(3-Acetyl-3-aza-bicyclo[3.1.0]hex-6-ylamino)-6-(2-chlorophenyl)-8H-pyrido[2,3-d]pyrimidin-7-one (Compound No. 48)
2-(3-Benzoyl-3-aza-bicyclo[3.1.0]hex-6-ylamino)-6-(2-chloro-phenyl)-8-(4-fluoro-phenyl)-8H-pyrido[2,3-d]pyrimidin-7-one (Compound No. 49);
2-(9-Benzyl-9-aza-bicyclo[3.3.1]non-3-ylamino)-6-(2-chloro-phenyl)-8-(4-fluoro-phenyl)-8H-pyrido[2,3-d]pyrimidin-7-one (Compound No. 50);
2-(9-Benzyl-9-aza-bicyclo[3.3.1]non-3-ylamino)-6-(2-chlorophenyl)-8-methyl-8H-pyrido[2,3-d]pyrimidin-7-one (Compound No. 51);
2-(7-Benzyl-3-oxa-7-aza-bicyclo[3.3.1]non-9-ylamino)-6-(2-chloro-phenyl)-8-(4-fluoro-phenyl)-8H-pyrido[2,3-d]pyrimidin-7-one (Compound No. 52);
2-(7-Benzyl-3-oxa-7-aza-bicyclo[3.3.1]non-9-ylamino)-6-(2-chloro-phenyl)-8-methyl-8H-pyrido[2,3-d]pyrimidin-7-one (Compound No. 53);
2-(8-Benzyl-8-aza-bicyclo[3.2.1]oct-3-ylamino)-6-(2-chloro-phenyl)-8-(4-fluoro-phenyl)-8H-pyrido[2,3-d]pyrimidin-7-one (Compound No. 54);
2-(8-Benzyl-8-aza-bicyclo[3.2.1]oct-3-ylamino)-6-(2-chloro-phenyl)-8-methyl-8H-pyrido[2,3-d]pyrimidin-7-one (Compound No. 55);
[2-(3-Benzyl-3-aza-bicycyclo[3.1.0]hex-6-ylamino)-6-(2-chloro-phenyl)-7-oxo-7H-pyrido[2,3-d]pyrimidin-8-yl]-acetonitrile. (Compound No. 56);
6-[6-(2-Chlorophenyl)-8-cyanomethyl-7-oxo-7,8-dihydro-pyrido[2,3-d]pyrimidin-2-ylamino]-3-aza-bicyclo[3.1.0]hexane-3-carboxylic acid (4-fluorophenyl)-amide (Compound No. 57);
2-(3-Aza-bicyclo[3.1.0]hex-6-ylamino)-6-(2-chloro-phenyl)-8H-pyrido[2,3-d]pyrimidin-7-one. (Compound No. 58);
2-(8-Benzyl-8-aza-bicyclo[3.2.1]oct-3-ylamino)-6-(2-chloro-phenyl)-8H-pyrido[2,3-d]pyrimidin-7-one (Compound No. 59);
2-(7-Benzyl-3-oxa-7-aza-bicyclo[3.3.1]non-9-ylamino}-6-(2-chloro-phenyl)-8H-pyrido[2,3-d]pyrimidin-7-one (Compound No. 60);
2-{7-Benzyl-3-oxa-7-aza-bicyclo[3.3.1]non-9-ylamino}-6-(2-chloro-phenyl)-8H-pyrido[2,3-d]pyrimidin-7-one (Compound No. 61);
2-(3-Benzyl-3-aza-bicyclo[3.1.0]hex-6-ylamino)-6-(2-chloro-phenyl)-8-(1H-tetrazol-5-ylmethyl)-8H-pyrido[2,3-d]pyrimidin-7-one (Compound No. 62);
6-[6-(2-Chloro-phenyl)-8-cyclopropyl-7-oxo-7,8-dihydro-pyrido[2,3-d]pyrimidin-2-ylamino]-3-aza-bicyclo[3.1.0]hexane-3-carboxylic acid amide (Compound No. 63);
3-{6-[6-(2-Chloro-phenyl)-8-cyclopropyl-7-oxo-7,8-dihydro-pyrido[2,3-d]pyrimidin-2-ylamino]-3-aza-bicyclo[3.1.0]hex-3-yl}-propionitrile (Compound No. 64);
9-[6-(2-chloro-phenyl)-8-cyclopropyl-7-oxo-7,8-dihydro-pyrido[2,3-d]pyrimidin-2-ylamino]-3-oxa-7-aza-bicyclo[3.3.1]nonane-7-carboxylic acid benzyl ester (Compound No. 65);
6-[6-(2-Chloro-phenyl)-8-cyclopropyl-7-oxo-7,8-dihydro-pyrido[2,3-d]pyrimidin-2-ylamino]-3-aza-bicyclo[3.1.0]hexane-3-carboxylic acid ethyl ester (Compound No. 66);
3-{6-[6-{2-Chloro-phenyl}-8-methyl-7-oxo-7,8-dihydro-pyrido[2,3-d]pyrimidin-2-ylamino]-3-aza-bicyclo[3.1.0]hex-3-yl}-propionitrile (Compound No. 67)
6-[6-(2-Chloro-phenyl)-8-methyl-7-oxo-7,8-dihydro-pyrido[2,-d]pyrimidin-2-ylamino]-3-aza-bicyclo[3.1.0]hexane-3-carboxylic acid ethyl ester (Compound No. 68);
6-[6-(2-Chloro-phenyl)-7-oxo-7,8-dihydro-pyrido[2,3-d]pyrimidin-2-ylamino]-3-aza-bicyclo[3.1.0]hexane-3-carboxylic acid benzyl ester (Compound No. 69);
2-(3-Acetyl-3-aza-bicyclo[3.1.0]hex-6-ylamino)-6-(2-chloro-phenyl)-8-(2-diethylamino-ethyl)-8H-pyrido[2,3-d]pyrimidin-7-one (Compound No. 70);
3-[2-(3-Benzyl-3-aza-bicyclo[3.1.0]hex-6-ylamino}-6-(2-chloro-phenyl)-7-oxo-7H-pyrido[2,3-d]pyrimidin-8-yl]-pyrrolidine-1-carboxylic acid tert-butyl ester (Compound No. 71);
2-(3-Benzyl-3-aza-bicyclo[3.1.0]hex-6-ylamino)-6-(2-chloro-phenyl)-8-(2-morpholin-4-yl-ethyl)-8H-pyrido[2,3-d]pyrimidin-7-one (Compound No. 72);
2-(3-Benzyl-3-aza-bicyclo[3.1.0]hex-6-ylamino)-6-(2-chlorophenyl)-8-(2-diethylaminoethyl)-8H-pyrido[2,3-d]pyrimidin-7-one (Compound No. 73);
2-(3-Benzyl-3-aza-bicyclo[3.1.0]hex-6-ylamino}-6-(2-chloro-phenyl)-8-(1-methyl-pyrrolidin-3-yl)-8H-pyrido[2,3-d]pyrimidin-7-one (Compound No. 74);
[2-(3-Benzyl-3-aza-bicyclo[3.1.0]hex-6-ylamino)-6-(2-chloro-phenyl)-7-oxo-7H-pyrido[2,3-d]pyrimidin-8-yl]-acetic acid ethyl ester (Compound No. 75);
[2-(3-Benzyl-3-aza-bicyclo[3.1.0]hex-6-ylamino)-6-(2-chloro-phenyl)-7-oxo-7H-pyrido[2,3-d]pyrimidin-8-yl]-acetic acid (Compound No. 76);
2-(3-Benzyl-3-aza-bicyclo[3.1.0]hex-6-ylamino)-6-(2-chlorophenyl)-8-pyrrolidin-3yl-8H-pyrido[2,3-d]pyrimidin-7-one (Compound No. 77);
2-(3-Acetyl-3-aza-bicyclo[3.1.0]hex-ylamine)-6-(2-chlorophenyl)-8H-pyrido[2,3-]pyrimidin-7-one (Compound No. 78);
2-[2-(3-Benzyl-3-aza-bicyclo[3.1.0]hex-6-ylamino)-6-(2-chloro-phenyl)-7-oxo-7H-pyrido[2,3-d]pyrimidin-8-yl]-acetamide (Compound No. 79);
[4-({6-[6-(2-Chloro-phenyl)-8-methyl-7-oxo-7,8-dihydro-pyrido[2,3-d]pyrimidin-2-ylamino]-3-aza-bicyclo[3.1.0]hexane-3-carbonyl]-amino)-phenyl]-acetic acid (Compound No. 80);
[4-({6-[6-(2-Chloro-phenyl)-8-methyl-7-oxo-7,8-dihydro-pyrido[2,3-d]pyrimidin-2-ylamino]-3-aza-bicyclo[3.1.0]hexane-3-carbonyl]-amino)-phenyl]-acetic acid (Compound No. 81), and
pharmaceutically acceptable salts, pharmaceutically acceptable solvates, esters, enantiomers, diastereomers, N-oxides, polymorphs or metabolites thereof.

18. A pharmaceutical composition comprising a therapeutically effective amount of one or more compounds of Formula I 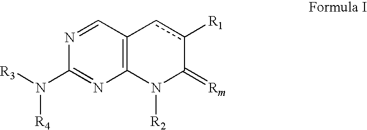
and its one or more pharmaceutically acceptable salts, pharmaceutically acceptable solvates, esters, enantiomers, diastereomers, N-oxides, polymorphs or metabolites;
wherein
R₁ is alkyl, alkenyl, alkynyl, cycloalkyl, aryl, aralkyl, heteroaryl, heterocyclyl, heteroarylalkyl, or heterocyclylalkyl;
when Rₘ is oxygen or sulphur, R₂ is alkyl, alkenyl, alkynyl, cycloalkyl, aryl, aralkyl, heteroaryl, heterocyclyl, heterocyclylalkyl or heteroarylalkyl;
when Rₘ is —NH, —N-acyl, —N(CN), —N(NO₂), —C(R₃)₂ or —CH(NO₂), R₂ is hydroxy, alkoxy, aryloxy, —CHO, —CN, alkyl, alkenyl, alkynyl, cycloalkyl, carboxy, halogen, aryl, aralkyl, acyl, heteroaryl, heterocyclyl, —SO₂R₅, —COOR₆, —C(═O)NRₓR_{y}, —NRₓR_{y} or —OC(═O)NRₓR_{y}, —NHC(═O)Rₓ;
the symbol [symbol] represents a single bond or a double bond;
R₃ is hydrogen, alkyl, alkenyl, alkynyl, cycloalkyl, aryl, heterocyclyl, heteroaryl, aralkyl, heteroarylalkyl or heterocyclylalkyl;
R₄ is 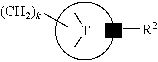
(wherein 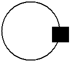
represents a cyclic ring having 4 or 5 carbon atoms, k is an integer selected from 0-2, M is O or N, and T is —(CH₂)ₙ—, —CH(O)CH₂—, —CH₂CH(O)CH₂—, —CH(O)—, —CH₂—O—CH₂—, —CH₂—NH—CH₂—);
R_{z} is no atom (when M is oxygen), hydrogen or Rᵤ (wherein Rᵤ is hydroxy, alkoxy, aryloxy, —CHO, —CN, alkyl, alkenyl, alkynyl, cycloalkyl, carboxy, halogen, aryl, aralkyl, acyl, heteroaryl, heterocyclyl, —SO₂R₅, —COOR₆, —C(═O)NRₓR_{y}, —NRₓR_{y} or —OC(═O)NRₓR_{y} or —NHC(═O)Rₓ);
n is an integer selected from 0-3 (wherein when n is zero then T represents a direct bond);
R₅ is alkyl, alkenyl, alkynyl, cycloalkyl, —NRₚR_{q} (wherein Rₚ and R_{q} is hydrogen, alkyl, alkenyl, alkynyl, cycloalkyl, aryl, aralkyl, heterocyclyl, heteroaryl, heterocyclylalkyl or heteroarylalkyl, or Rₚ and R_{q} may also together join to form a heterocyclyl ring), aryl, aralkyl, heteroaryl, heterocyclyl, heterocyclylalkyl or heteroarylalkyl;
R₆ is alkyl, alkenyl, alkynyl, cycloalkyl, aryl, aralkyl, heteroarylalkyl or heterocyclylalkyl;
Rₓ and R_{y} are independently hydrogen, alkyl, alkenyl, alkynyl, cycloalkyl, aryl, aralkyl, —SO₂R₅ (wherein R₅ is the same as defined above), heteroaryl, heterocyclyl, heteroarylalkyl or heterocyclylalkyl;
Q is alkyl, alkenyl, alkynyl, cycloalkyl, aryl, heteroaryl, heterocyclyl, aralkyl, heteroarylalkyl or heterocyclylalkyl.
together with one or more pharmaceutically acceptable carriers, excipients or diluents.

19. A method for the treatment or prophylaxis of inflammatory diseases or associated pathologies in an animal or a human suffering therefrom which comprises administering to the mammal an effective amount of one or more compounds of Formula I: 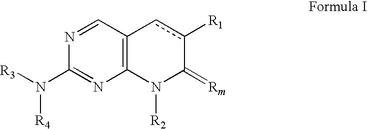
and its pharmaceutically acceptable salts, pharmaceutically acceptable solvates, esters, enantiomers diastereomers, N-oxides, polymorphs, metabolites;
wherein
R₁ is alkyl, alkenyl, alkynyl, cycloalkyl, aryl, aralkyl, heteroaryl, heterocyclyl, heteroarylalkyl, or heterocyclylalkyl;
when Rₘ is oxygen or sulphur, R₂ is alkyl, alkenyl, alkynyl, cycloalkyl, aryl, aralkyl, heteroaryl, heterocyclyl, heterocyclylalkyl or heteroarylalkyl;
when Rₘ is —NH, —N-acyl, —N(CN), —N(NO₂), —C(R₃)₂ or —CH(NO₂), R₂ is hydroxy, alkoxy, aryloxy, —CHO, —CN, alkyl, alkenyl, alkynyl, cycloalkyl, carboxy, halogen, aryl, aralkyl, acyl, heteroaryl, heterocyclyl, —SO₂R₅, —COOR₆, —C(═O)NRₓR_{y}, —NRₓR_{y} or —OC(═O)NRₓR_{y}, —NHC(═O)Rₓ;
the symbol [symbol] represents a single bond or a double bond;
R₃ is hydrogen, alkyl, alkenyl, alkynyl, cycloalkyl, aryl, heterocyclyl, heteroaryl, aralkyl, heteroarylalkyl or heterocyclylalkyl;
R₄ is 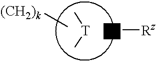
(wherein 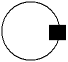
represents a cyclic ring having 4 or 5 carbon atoms, k is an integer selected from 0-2, M is O or N, and T is —(CH₂)ₙ—, —CH(O)CH₂—, —CH₂CH(O)CH₂—, —CH(O)—, —CH₂—O—CH₂—, —CH₂—NH—CH₂—);
R_{z} is no atom (when M is oxygen), hydrogen or Rᵤ (wherein Rᵤ is hydroxy, alkoxy, aryloxy, —CHO, —CN, alkyl, alkenyl, alkynyl, cycloalkyl, carboxy, halogen, aryl, aralkyl, acyl, heteroaryl, heterocyclyl, —SO₂R₅, —COOR₆, —C(═O)NRₓR_{y}, —NRₓR_{y} or —OC(═O)NRₓR_{y} or —NHC(═O)Rₓ);
n is an integer selected from 0-3 (wherein when n is zero then T represents a direct bond);
R₅ is alkyl, alkenyl, alkynyl, cycloalkyl, —NRₚR_{q} (wherein Rₚ and R_{q} is hydrogen, alkyl, alkenyl, alkynyl, cycloalkyl, aryl, aralkyl, heterocyclyl, heteroaryl, heterocyclylalkyl or heteroarylalkyl, or Rₚ and R_{q} may also together join to form a heterocyclyl ring), aryl, aralkyl, heteroaryl, heterocyclyl, heterocyclylalkyl or heteroarylalkyl;
R₆ is alkyl, alkenyl, alkynyl, cycloalkyl, aryl, aralkyl, heteroarylalkyl or heterocyclylalkyl;
Rₓ and R_{y} are independently hydrogen, alkyl, alkenyl, alkynyl, cycloalkyl, aryl, aralkyl, —SO₂R₅ (wherein R₅ is the same as defined above), heteroaryl, heterocyclyl, heteroarylalkyl or heterocyclylalkyl;
Q is alkyl, alkenyl, alkynyl, cycloalkyl, aryl, heteroaryl, heterocyclyl, aralkyl, heteroarylalkyl or heterocyclylalkyl.

20. A method according to claim 19, wherein the inflammatory disease or associated pathology includes sepsis, rheumatoid arthritis, inflammatory bowel disease, type-1 diabetes, asthma, chronic obstructive pulmonary disorder, organ transplant rejection or psoriasis.

21. A process for preparing a compound of Formula XI, or its pharmaceutically acceptable salts, pharmaceutically acceptable solvates, enantiomers, diastereomers, polymorphs or N-oxides, 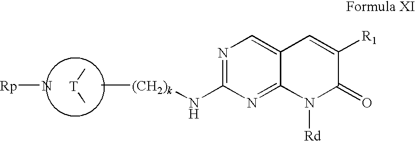
wherein
R′ is alkyl;
R_{d} is hydrogen, optionally substituted alkyl, cycloalkyl or aryl;
Rₚ is alkyl, aralkyl, —C(═O)NRₓR_{y} or —C(═O)OCH₂C₆H₅;
k is an integer selected from 0-2;
T is —(CH₂)ₙ—, —CH(O)CH₂—, —CH₂CH(O)CH₂—, —CH(O)—, —CH₂—O—CH₂—, —CH₂—NH—CH₂—) wherein Q is alkyl, alkenyl, alkynyl, cycloalkyl, aryl, heteroaryl, heterocyclyl, aralkyl, heteroarylalkyl or heterocyclylalkyl; and
R₁ is alkyl, alkenyl, alkynyl, cycloalkyl, aryl, aralkyl, heteroaryl, heterocyclyl, heteroarylalkyl or heterocyclylalkyl,
wherein the method comprises steps of:
a. reacting a compound of Formula II 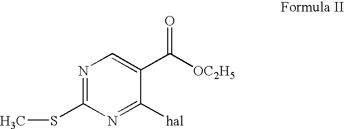
with a compound of Formula III 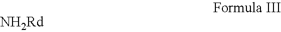
to give a compound of Formula IV; 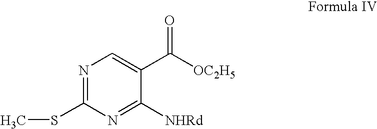
b. reducing a compound of Formula IV to give a compound of Formula V; 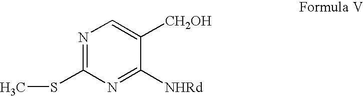
c. oxidizing a compound of Formula V to give a compound of Formula VI; 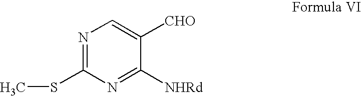
d. reacting a compound of Formula VI with an ester of Formula VII 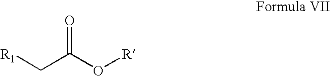
to give a compound of Formula VIII; 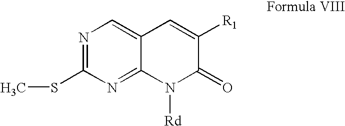
e. oxidizing a compound of Formula VIII to give a compound of Formula IX; and 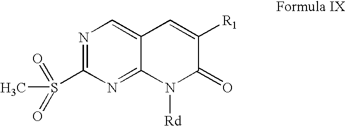
f. reacting a compound of Formula IX with a compound of Formula X 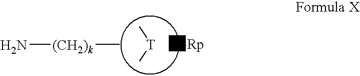
to give a compound of Formula XI.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)
